(19) United States
(12) Patent Application Publication
(10) Pub. No.: US 2004/0058969 A1
(43) Pub. Date: Mar. 25, 2004

(54) Novel epothilone derivatives, method for the preparation thereof and their pharmaceutical use

(76) Inventors: Bernd Buchmann, Hohen Neuendorf (DE); Ulrich Klar, Berlin (DE); Werner Skuballa, Berlin (DE); Wolfgang Schwede, Berlin (DE); Rosemarie Lichtner, Berlin (DE); Jens Hoffmann, Muehlenbeck (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/257,925

(22) PCT Filed: Apr. 19, 2001
(86) PCT No.: PCT/EP01/04552

(30) Foreign Application Priority Data
Apr. 19, 2000 (DE) 100 20 517.8

(51) Int. Cl. A61K 31/427; C07D 417/02

(52) U.S. Cl. 514/365; 548/181

(57) ABSTRACT

The invention relates to novel epothilone derivatives of general formula (1), wherein R⁵ represents a halogen atom or a cyano group and the other substituents have the meanings as cited in the description. The novel compounds interact with tubulin by stabilizing formed microtubules. They are capable of influencing the cell division in a phase-specific manner and are well-suited for the treatment of malignant tumors, for example, ovarian, stomach, colon, adeno, breast, lung, head and neck carcinomas, malignant melanoma, acute lymphocytic and myelocytic leukemia. In addition, they are well-suited for anti-angiogenesis therapy and for the treatment of chronic inflammable medical disorders (psoriasis, arthritis). In order to prevent uncontrolled cell growths as well as to improve the compatibility of medical implants, the inventive epothilone derivatives can be applied to or introduced into polymeric materials. The inventive compounds can be used alone or in order to obtain additive or synergistic effects, in conjunction with additional principles and substance classes that can be used in tumor therapy.

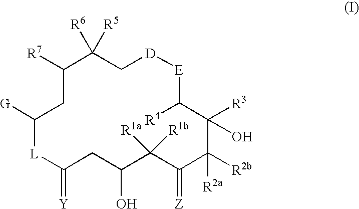

Höfle et al. describe the cytotoxic action of the natural products epothilone A (R=hydrogen) and epothilone B (R=methyl)

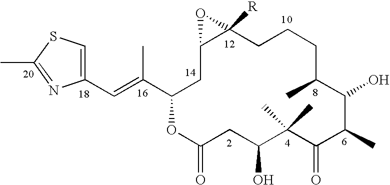

Epothilone A (R═H), Epothilone B (R═CH₃) in, e.g., Angew. Chem. [Applied Chem.] 1996, 108, 1671-1673. Because of their in-vitro selectivity for breast cell lines and intestinal cell lines and their significantly higher activity against P-glycoprotein-forming multiresistant tumor lines in comparison to taxol as well as their physical properties that are superior to those of taxol, e.g., a water solubility that is higher by a factor of 30, this novel structural class is especially advantageous for the development of a pharmaceutical agent for treating malignant tumors.

The natural products are not sufficiently stable either chemically or metabolically for the development of pharmaceutical agents. To eliminate these drawbacks, modifications to the natural product are necessary. Such modifications are possible only with a total-synthesis approach and require synthesis strategies that make possible a broad modification of the natural product. The purpose of the structural changes is also to increase the therapeutic range. This can be done by improving the selectivity of the action and/or increasing the active strength and/or reducing undesirable toxic side effects, as they are described in Proc. Natl. Acad. Sci. USA 1998, 95, 9642-9647.

The total synthesis of epothilone A is described by Schinzer et al. in Chem. Eur. J. 1996, 2, No. 11, 1477-1482 and in Angew. Chem. 1997, 109, No. 5, pp. 543-544).

Epothilone derivatives were already described by Höfle et al. in WO 97/19086. These derivatives were produced starting from natural epothilone A or B. Also, epothilone C and D (double bond between carbon atoms 12 and 13: epothilone C=deoxyepothilone A; Epothilone D=deoxyepothilone B) are described as possible starting products in this respect.

Another synthesis of epothilone and epothilone derivatives was described by Nicolaou et al. in Angew. Chem. 1997, 109, No. 1/2, pp. 170-172. The synthesis of epothilones A and B and several epothilone analogs was described in Nature, Vol. 387, 1997, pp. 268-272; and the synthesis of epothilone A and its derivatives was described in J. Am. Chem. Soc., Vol. 119, No. 34, 1997, pp. 7960-7973 as well as the synthesis of epothilones A and B and several epothilone analogs in J. Am. Chem. Soc., Vol. 119, No. 34, 1997, pp. 7974-7991 also by Nicolaou et al.

Nicolaou et al. also describe in Angew. Chem. 1997, 109, No. 19, pp. 2181-2187 the production of epothilone A analogs using combinatory solid-phase synthesis. Several epothilone B analogs are also described there.

Epothilone derivatives, in some cases also epothilones C and D, are described in addition in Patent Applications WO 99/07692, WO 99/02514, WO 99/01124, WO 99/67252, WO 98/25929, WO 97/19086, WO 98/38192, WO 99/22461 and WO 99/58534.

In the epothilone derivatives that have become known to date, no halogen atom nor cyano group can be at carbon atom 12 of the epothilone skeleton.

The object of this invention consists in making available new epothilone derivatives, which are both chemically and metabolically stable enough for the development of pharmaceutical agents and which are superior to natural derivatives in terms of their therapeutic range, their selectivity of action and/or undesirable toxic side effects and/or their active strength.

This invention describes the new epothilone derivatives of general formula I,

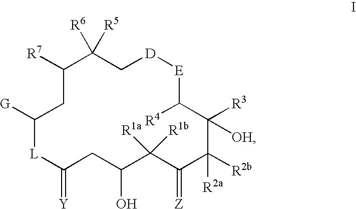

in which

R^{1a}, R^{1b} are the same or different and mean hydrogen, C₁-C₁₀ alkyl, aryl, C₇-C₂₀ aralkyl, or together a —(CH₂)ₘ group with m=2, 3, 4 or 5, or a —CH₂—O—CH₂ group,

R^{2a}, R^{2b} are the same or different and mean hydrogen, C₁-C₁₀-alkyl, aryl, C₇-C₂₀-aralkyl, —(CH₂)ᵣ—C≡C—(CH₂)ₚ—R²⁰, —(CH₂)ᵣCH═CH—(CH₂)ₚ—R²⁰,

r are the same or different and mean 0 to 4,

p are the same or different and mean 0 to 3,

R²⁰ is equal to hydrogen, C₁-C₁₀-alkyl, aryl, C₇-C₂₀-aralkyl, C₁-C₁₀-acyl, or if p>0, a group OR²¹,

R²¹ means hydrogen, or a protective group PG6,

R³ means hydrogen, C₁-C₁₀-alkyl, aryl, C₇-C₂₀-aralkyl,

R⁴ means hydrogen, C₁-C₁₀-alkyl, aryl, C₇-C₂₀-aralkyl,

D means oxygen, sulfur, sulfoxide or sulfone, whereby then E must be equal to methylene or

D-E together mean a group

H₂C—CH₂, HC═CH C≡C

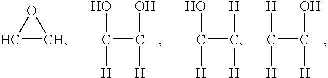

R⁵ means halogen or cyano,

R⁶, R⁷ together mean an additional bond or an oxygen atom,

G means a bicyclic or tricyclic aryl radical or the group

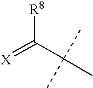

whereby R⁸ means hydrogen, fluorine, chlorine, bromine, cyano, C₁-C₂₀-alkyl, aryl, C₇-C₂₀-aralkyl, which can all be substituted,

X means an oxygen atom, two alkoxy groups OR¹⁹, a C₂-C₁₀-alkylene-α,ω-dioxy group, which can be straight-chain or branched, H/OR⁹ or a grouping CR¹⁰R¹¹,

whereby

R¹⁹ stands for a C₁-C₂₀-alkyl radical,

R⁹ stands for hydrogen or a protective group PG^{x},

R¹⁰, R¹¹ are the same or different and stand for hydrogen, a C₁-C₂₀-alkyl, aryl, C₇-C₂₀-aralkyl radical or R¹⁰ and R¹¹ together with the methylene carbon atom together stand for a 5- to 7-membered carbocyclic ring,

L means oxygen or NR²², whereby R²² is a hydrogen atom or a C₁-C₂₀-alkyl radical,

Y means an oxygen atom or two hydrogen atoms,

Z means an oxygen atom or H/OR

whereby

R¹² means hydrogen or a protective group PG^{z}.

The production of the new epothilone derivatives is based on the linkage of two partial fragments A and B. The interfaces are as indicated in general formula I′.

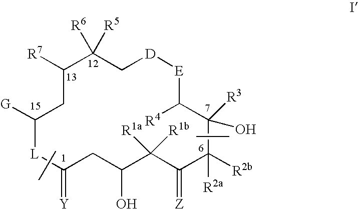

A means a C₁-C₆ fragment (epothilone numbering system) of general formula

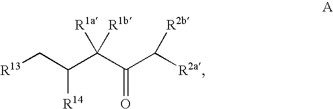

in which

R^{1a′}, R^{1b′}, R^{2a′} and R^{2b′} have the meanings already mentioned for R^{1a}, R^{1b}, R^{2a} and R^{2b}, and

R¹³ means CH₂OR¹³, CH₂-Hal, CHO, CO₂R^{13b}, COHal,

R¹⁴ means hydrogen, OR^{14a}, Hal, OSO₂R^{14b},

R^{13a}, R^{14a} mean hydrogen, SO₂-alkyl, SO₂-aryl, SO₂-aralkyl or together a —(CH₂)ₒ group or together a CR^{15a}R^{15b} group,

R^{13b}, R^{14b} mean hydrogen, C₁-C₂₀-alkyl, aryl, or C₁-C₂₀-aralkyl,

R^{15a}, R^{15b} are the same or different and mean hydrogen, C₁-C₁₀-alkyl, aryl, C₇-C₂₀-aralkyl or together a —(CH₂)_{q} group,

Hal means halogen,

0 means 2 to 4,

q means 3 to 6,

including all stereoisomers as well as their mixtures, and

free hydroxyl groups in R¹³ and R¹⁴ can be etherified or esterified, free carbonyl groups can be ketalized in A and R¹³, converted into an enol ether or reduced, and free acid groups in A can be converted into their salts with bases.

B stands for a C7-C15 fragment (epothilone numbering system) of general formula

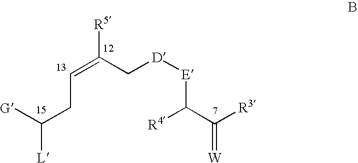

in which

R^{3′},R^{4′}, R^{5′}, D′, E′ and G′ have the meanings already mentioned for R³, R⁴, R⁵, D, E and G, and

W means an oxygen atom, two alkoxy groups OR¹⁷, a C₂-C₁₀-alkylene-α,ω-dioxy group, which can be straight-chain or branched or H/OR¹⁶,

R¹⁶ means hydrogen or a protective group PG¹

R¹⁷ means C₁-C₂₀-alkyl,

R¹⁸ means a hydrogen atom or a protective group PG²

L′ means an azide or the group OR²³, whereby R²³ means a hydrogen or a protective group PG¹⁰.

As alkyl groups R^{1a}, R^{1b}, R^{2a}, R^{2b}, R³, R⁴, R⁸, R⁹, R¹⁰, R¹¹, R¹², R^{13b}, R^{14b}, R^{15a}, R^{15b}, R¹⁷, R¹⁹ and R²², straight-chain or branched-chain alkyl groups with 1-20 carbon atoms can be considered, such as, for example, methyl, ethyl, propyl, isopropyl, butyl, isobutyl, tert-butyl, pentyl, isopentyl, neopentyl, heptyl, hexyl, and decyl.

Alkyl groups R^{1a}, R^{1b}, R^{2a}, R^{2b}, R³, R⁴, R⁸,R⁹, R¹⁰, R¹¹, R¹², R^{13b}, R^{14b}, R^{15a}, R^{15b}, R¹⁷, R¹⁹ and R²² can be perfluorinated or substituted by 1-5 halogen atoms, hydroxy groups, C₁-C₄-alkoxy groups, or C₆-C₁₂-aryl groups (which can be substituted by 1-3 halogen atoms).

As aryl radicals R^{1a}, R^{1b}, R^{2a}, R^{2b}, R³, R⁴, R⁸,R⁹, R¹⁰, R¹¹, R¹², R^{13b}, R^{14b}, R^{15a} and R^{15b}, substituted and unsubstituted carbocyclic or heterocyclic radicals with one or more heteroatoms, such as, e.g., phenyl, naphthyl, furyl, thienyl, pyridyl, pyrazolyl, pyrimidinyl, oxazolyl, pyridazinyl, pyrazinyl, quinolyl, thiazolyl, which can be substituted in one or more places by halogen, OH, O-alkyl, CO₂H, CO₂-alkyl, —NH₂, —NO₂, —N₃, —CN, C₁-C₂₀-alkyl, C₁-C₂₀-acyl, or C₁-C₂₀-acyloxy groups, are suitable.

The aralkyl groups in R^{1a}, R^{1b}, R^{2a}, R^{2b}, R³, R⁴, R⁸, R⁹, R¹⁰, R¹¹, R¹², R^{13b}, R^{14b}, R^{15a} and R^{15b} can contain in the ring up to 14 C atoms, preferably 6 to 10, and in the alkyl chain 1 to 8, preferably 1 to 4 atoms. As aralkyl radicals, for example, benzyl, phenylethyl, naphthylmethyl, naphthylethyl, furylmethyl, thienylethyl, and pyridylpropyl are suitable. The rings can be substituted in one or more places by halogen, OH, O-alkyl, CO₂H, CO₂-alkyl, —NO₂, —N₃, —CN, C₁-C₂₀-alkyl, C₁-C₂₀-acyl, or C₁-C₂₀-acyloxy groups.

For the bicyclic and tricyclic aryl radicals that are contained in G in general formula I, substituted and unsubstituted carbocyclic or heterocyclic radicals with one or more heteroatoms, such as, e.g., naphthyl, anthryl, benzothiazolyl, benzoxazolyl, benzimidazolyl, quinolyl, isoquinolyl, benzoxazinyl, benzofuranyl, indolyl, indazolyl, quinoxalinyl, tetrahydroisoquinolinyl, tetrahydroquinolinyl, thienopyridinyl, pyridopyridinyl, benzopyrazolyl, benzotriazolyl, dihydroindolyl, which can be substituted in one or more places by halogen, OH, O-alkyl, CO₂H, CO₂-alkyl, —NH₂, —NO₂, —N₃, —CN, C₁-C₂₀-alkyl, C₁-C₂₀-acyl, or C₁-C₂₀-acyloxy groups, are suitable.

The alkoxy groups that are contained in X in general formula I are to contain 1 to 20 carbon atoms in each case, whereby methoxy, ethoxy, propoxy, isopropoxy and t-butyloxy groups are preferred.

As representatives of protective groups PG, alkyl- and/or aryl-substituted silyl, C₁-C₂₀-alkyl, C₄-C₇-cycloalkyl, which in addition in the ring can contain an oxygen atom, aryl, C₇-C₂₀-aralkyl, C₁-C₂₀-acyl as well as aroyl can be mentioned.

As alkyl, silyl and acyl radicals for protective groups PG, the radicals that are known to one skilled in the art are suitable. Preferred are alkyl or silyl radicals that can be easily cleaved from the corresponding alkyl and silyl ethers, such as, for example, the trityl, dimethoxytrityl, methoxymethyl, methoxyethyl, ethoxyethyl, tetrahydropyranyl, tetrahydrofuranyl, trimethylsilyl, triethylsilyl, tert-butyldimethylsilyl, tert-butyldiphenylsilyl, tribenzylsilyl, triisopropylsilyl, benzyl, para-nitrobenzyl, para-methoxybenzyl radicals, trityl, dimethoxytrityl, as well as alkylsulfonyl and arylsulfonyl radicals. As acyl radicals, e.g., formyl, acetyl, propionyl, isopropionyl, pivalyl, butyryl, trichloromethoxycarbonyl or benzoyl, which can be substituted with amino and/or hydroxy groups, are suitable.

As amino protective groups, the radicals that are known to one skilled in the art are suitable. For example, the alloc, boc, Z, benzyl, f-moc, troc, stabase or benzostabase groups can be mentioned.

Acyl groups PG^{x} or PG^{z} in R⁹ and R¹² can contain 1 to 20 carbon atoms, whereby formyl, acetyl, propionyl, isopropionyl and pivalyl groups are preferred.

Index m in the alkylene group that is formed from R^{1a} and R^{1b} preferably stands for 2, 3 or 4.

The C₂-C₁₀-alkylene-α,ω-dioxy group that is possible for X is preferably an ethyleneketal or neopentylketal group.

According to a variant of the invention, R^{1a} and R^{1b} each stand for a methyl group or together for an ethylene or trimethylene group.

Z primarily stands for an oxygen atom.

The two substituents R^{2a} and R^{2b} are selected according to another variant such that one stands for a hydrogen atom and the other stands for a methyl, ethyl, propyl, butyl, benzyl, allyl, homoallyl, propargyl or homopropargyl group.

R³ preferably stands for a hydrogen atom.

Another embodiment calls for R⁴ to stand for a methyl, ethyl, propyl, butyl or benzyl group.

In another variant, D stands for an oxygen atom, and E stands for a methylene group, or D and E together stand for an ethylene group.

Substituent R⁵ is preferably a fluorine, chlorine or bromine atom, especially a fluorine or chlorine atom.

According to another variant, G stands for a bicyclic heteroaryl radical with at least one nitrogen atom; in this case, it is preferably a 2-methyl-5-benzothiazolyl radical or a 2-methyl-5-benzoxazolyl radical.

In another variant, G stands for the grouping X═C(R⁸)—, in which R⁸ is a hydrogen, fluorine, chlorine or bromine atom, or a methyl group, and X is a group ═CR¹⁰R¹¹, in which R¹⁰ stands for a hydrogen atom and R¹¹ stands for a heteroaryl radical, or vice versa.

The heteroaryl radical is primarily a 2-methyl-4-thiazolyl, 2-pyridyl or 2-methyl-4-oxazolyl radical.

Finally, L and Y can preferably be selected such that a lactone group or lactam group is formed in the epothilone skeleton, i.e., L is an oxygen atom or a nitrogen group —NR²²— with R²² in the meaning of a hydrogen atom or a methyl or ethyl group, and Y stands for an oxygen atom.

The compounds that are mentioned below are preferred according to the invention:

(4S,7R,8S,9S,13E/Z,16S(E))-4,8-Dihydroxy-13-fluoro-16-(1-methyl-2-(2-methyl-4-thiazolyl)ethenyl)-1,11-dioxa-5,5,7,9-tetramethyl-cyclohexadec-13-ene-2,6-dione

(1S/R,3S(E),7S,10R,11S,12S,16S/R)-16-Fluoro-7,11-dihydroxy-3-(1-methyl-2-(2-methyl-4-thiazolyl)ethenyl)-8,8,10,12-tetramethyl-4,14,17-trioxabicyclo[14.1.0]heptadecane-5,9-dione

(4S,7R,8S,9S,13E/Z,16S(E))-4,8-Dihydroxy-13-chloro-16-(1-methyl-2-(2-methyl-4-thiazolyl)ethenyl)-1,11-dioxa-5,5,7,9-tetraamethyl-cyclohexadec-13-ene-2,6-dione

(1S/R,3S(E),7S,10R,11S,12S,16S/R)-16-Chloro-7,11-dihydroxy-3-(1-methyl-2-(2-methyl-4-thiazolyl)ethenyl)-8,8,10,12-tetramethyl-4,14,17-trioxabicyclo[14.1.0]heptadecane-5,9-dione

(4S,7R,8S,9S,13E/Z,16S(E))-4,8-Dihydroxy-13-cyano-16-(1-methyl-2-(2-methyl-4-thiazolyl)ethenyl)-1,11-dioxa-5,5,7,9-tetraamethyl-cyclohexadec-13-ene-2,6-dione

(1S/R,3S(E),7S,10R,11S,12S,16S/R)-16-Cyano-7,11-dihydroxy-3-(1-methyl-2-(2-methyl-4-thiazolyl)ethenyl)-8,8,10,12-tetramethyl-4,14,17-trioxabicyclo[14.1.0]heptadecane-5,9-dione

(4S,7R,8S,9S,13E/Z,16S(E))-4,8-Dihydroxy-7-ethyl-13-fluoro-16-(1-methyl-2-(2-methyl-4-thiazolyl)ethenyl)-1,11-dioxa-5,5,9-trimethyl-cyclohexadec-13-ene-2,6-dione

(1S/R,3S(E),7S,10R,11S,12S,16S/R)-16-Fluoro-10-ethyl-7,11-dihydroxy-3-(1-methyl-2-(2-methyl-4-thiazolyl)ethenyl)-8,8,12-trimethyl-4,14,17-trioxabicyclo[14.1.0]heptadecane-5,9-dione

(4S,7R,8S,9S,13E/Z,16S(E))-4,8-Dihydroxy-7-allyl-13-chloro-16-(1-methyl-2-(2-methyl-4-thiazolyl)ethenyl)-1,11-dioxa-5,5,9-trimethyl-cyclohexadec-13-ene-2,6-dione

(1S/R,3S(E),7S,10R,11S,12S,16S/R)-16-Chloro-10-allyl-7,11-dihydroxy-3-(1-methyl-2-(2-methyl-4-thiazolyl)ethenyl)-8,8,12-trimethyl-4,14,17-trioxabicyclo [14.1.0]heptadecane-5,9-dione

(4S,7R,8S,9S,13E/Z,16S(E))-4,8-Dihydroxy-13-chloro-16-(1-methyl-2-(2-pyridyl)ethenyl)-1,11-dioxa-5,5,7,9-tetraamethyl-cyclohexadec-13-ene-2,6-dione

(1S/R,3S(E),7S,10R,11S,12S,16S/R)-16-Chloro-7,11-dihydroxy-3-(1-methyl-2-(2-pyridyl)ethenyl)-8,8,10,12-tetramethyl-4,14,17-trioxabicyclo[14.1.0]heptadecane-5,9-dione

(4S,7R,8S,9S,13E/Z,16S(Z))-4,8-Dihydroxy-13-chloro-16-(1-fluoro-2-(2-methyl-4-thiazolyl)ethenyl)-1,11-dioxa-5,5,7,9-tetraamethyl-cyclohexadec-13-ene-2,6-dione (1S/R,3S(Z),7S,10R,11S,12S,16S/R)-16-Chloro-7,11-dihydroxy-3-(1-fluoro-2-(2-methyl-4-thiazolyl)ethenyl)-8,8,10,12-tetramethyl-4,14,17-trioxabicyclo[14.1.0]heptadecane-5,9-dione

(4S,7R,8S,9S,13E/Z,16S(Z))-4,8-Dihydroxy-13-chloro-16-(1-chloro-2-(2-methyl-4-thiazolyl)ethenyl)-1,11-dioxa-5,5,7,9-tetraamethyl-cyclohexadec-13-ene-2,6-dione

(1S/R,3S(Z),7S,10R,11S,12S,16S/R)-16-Chloro-7,11-dihydroxy-3-(1-chloro-2-(2-methyl-4-thiazolyl)ethenyl)-8,8,10,12-tetramethyl-4,14,17-trioxabicyclo[14.1.0]heptadecane-5,9-dione

(4S,7R,8S,9S,13E/Z,16S(Z))-4,8-Dihydroxy-13-chloro-16-(1-fluoro-2-(2-methyl-4-thiazolyl)ethenyl)-5,5-trimethylene-1,11-dioxa-7,9-dimethyl-cyclohexadec-13-ene-2,6-dione

(1S/R,3S(Z),7S,10R,11S,12S,16S/R)-16-Chloro-7,11-dihydroxy-3-(1-fluoro-2-(2-methyl-4-thiazolyl)ethenyl)-8,8-trimethylene-10,12-dimethyl-4,14,17-trioxabicyclo[14.1.0]heptadecane-5,9-dione

(4S,7R,8S,9S,13E/Z,16S(Z))-4,8-Dihydroxy-13-chloro-16-(1-chloro-2-(2-methyl-4-thiazolyl)ethenyl)-5,5-trimethylene-1,11-dioxa-7,9-dimethyl-cyclohexadec-13-ene-2,6-dione

(1S/R,3S(Z),7S,10R,11S,12S,16S/R)-16-Chloro-7,11-dihydroxy-3-(1-chloro-2-(2-methyl-4-thiazolyl)ethenyl)-8,8-trimethylene-10,12-dimethyl-4,14,17-trioxabicyclo[14.1.0]heptadecane-5,9-dione

(4S,7R,8S,9S,13E/Z,16S(Z))-4,8-Dihydroxy-13-chloro-16-(1-fluoro-2-(2-methyl-4-oxazolyl)ethenyl)-1,11-dioxa-5,5,7,9-tetraamethyl-cyclohexadec-13-ene-2,6-dione

(1S/R,3S(Z),7S,10R,11S,12S,16S/R)-16-Chloro-7,11-dihydroxy-3-(1-fluoro-2-(2-methyl-4-oxazolyl)ethenyl)-8,8,10,12-tetramethyl-4,14,17-trioxabicyclo[14.1.0]heptadecane-5,9-dione

(4S,7R,8S,9S,13E/Z,16S(Z))-4,8-Dihydroxy-13-chloro-16-(1-fluoro-2-(2-pyridyl)ethenyl)-1,11-dioxa-5,5,7,9-tetraamethyl-cyclohexadec-13-ene-2,6-dione

(1S/R,3S(Z),7S,10R,11S,12S,16S/R)-16-Chloro-7,11-dihydroxy-3-(1-fluoro-2-(2-pyridyl)ethenyl)-8,8,10,12-tetramethyl-4,14,17-trioxabicyclo[14.1.0]heptadecane-5,9-dione

(4S,7R,8S,9S,13E/Z,16S(Z))-4,8-Dihydroxy-7-ethyl-13-chloro-16-(1-chloro-2-(2-pyridyl)ethenyl)-1,11-dioxa-5,5,9-trimethyl-cyclohexadec-13-ene-2,6-dione

(1S/R,3S(Z),7S,10R,11S,12S,16S/R)-16-Chloro-7,11-dihydroxy-7-ethyl-3-(1-chloro-2-(2-pyridyl)ethenyl)-8,8,12-trimethyl-4,14,17-trioxabicyclo[14.1.0]heptadecane-5,9-dione

(4S,7R,8 S,9S,13E/Z,16S)-4,8-Dihydroxy-13-fluoro-16-(2-methyl-5-benzothiazolyl)-1,11-dioxa-5,5,7,9-tetramethyl-cyclohexadec-13-ene-2,6-dione

(1S/R,3S,7S,10R,11S,12S,16S/R)-16-Fluoro-7,11-dihydroxy-3-(2-methyl-5-benzothiazolyl)-8,8,10,12-tetramethyl-4,14,17-trioxabicyclo[14.1.0]heptadecane-5,9-dione

(4S,7R,8 S,9S,13E/Z,16S)-4,8-Dihydroxy-13-chloro-16-(2-methyl-5-benzothiazolyl)-1,11-dioxa-5,5,7,9-tetraamethyl-cyclohexadec-13-ene-2,6-dione

(1S/R,3S,7S,10R,11S,12S,16S/R)-16-Chloro-7,11-dihydroxy-3-(2-methyl-5-benzothiazolyl)-8,8,10,12-tetramethyl-4,14,17-trioxabicyclo[14.1.0]heptadecane-5,9-dione

(4S,7R,8S,9S,13E/Z,16S)-4,8-Dihydroxy-13-cyano-16-(2-methyl-5-benzothiazolyl)-1,11-dioxa-5,5,7,9-tetraamethyl-cyclohexadec-13-ene-2,6-dione

(1S/R,3 S,7S,10R,1 S,12S,16S/R)-16-Cyano-7,11-dihydroxy-3-(2-methyl-5-benzothiazolyl)-8,8,10,12-tetramethyl-4,14,17-trioxabicyclo[14.1.0]heptadecane-5,9-dione

(4S,7R,8S,9S,13E/Z,16S)-4,8-Dihydroxy-7-ethyl-13-chloro-16-(2-methyl-5-benzothiazolyl)-1,11-dioxa-5,5,9-trimethyl-cyclohexadec-13-ene-2,6-dione

(1S/R,3S,7S,10R,11S,12S,16S/R)-16-Chloro-7,11-dihydroxy-10-ethyl-3-(2-methyl-5-benzothiazolyl)-8,8,12-trimethyl-4,14,17-trioxabicyclo[14.1.0]heptadecane-5,9-dione

(4S,7R,8S,9S,13E/Z,16S)-4,8-Dihydroxy-7-allyl-13-chloro-16-(2-methyl-5-benzothiazolyl)-1,11-dioxa-5,5,9-trimethyl-cyclohexadec-13-ene-2,6-dione

(1S/R,3S,7S,10R,11S,12S,16S/R)-16-Chloro-7,11-dihydroxy-10-allyl-3-(2-methyl-5-benzothiazolyl)-8,8,12-trimethyl-4,14,17-trioxabicyclo[14.1.0]heptadecane-5,9-dione

(4S,7R,8S,9S,13E/Z,16S)-4,8-Dihydroxy-13-fluoro-16-(2-methyl-5-benzoxazolyl)-1,11-dioxa-5,5,7,9-tetramethyl-cyclohexadec-13-ene-2,6-dione

(1S/R,3S,7S,10R,11 S,12S,16S/R)-16-Fluoro-7,11-dihydroxy-3-(2-methyl-5-benzoxazolyl)-8,8,10,12-tetramethyl-4,14,17-trioxabicyclo[14.1.0]heptadecane-5,9-dione

(4S,7R,8S,9S,13E/Z,16S)-4,8-Dihydroxy-13-chloro-16-(2-methyl-5-benzoxazolyl)-1,11-dioxa-5,5,7,9-tetraamethyl-cyclohexadec-13-ene-2,6-dione

(1S/R,3 S,7S,10R,11 S,12S,16S/R)-16-Chloro-7,11-dihydroxy-3-(2-methyl-5-benzoxazolyl)-8,8,10,12-tetramethyl-4,14,17-trioxabicyclo [14.1.0]heptadecane-5,9-dione

(4S,7R,8S,9S,13E/Z,16S(E))-4,8-Dihydroxy-13-chloro-16-(1-methyl-2-(2-methyl-4-thiazolyl)ethenyl)-1-oxa-5,5,7,9-tetraamethyl-cyclohexadec-13-ene-2,6-dione

(1S/R,3S(E),7S,10R,11 S,12S,16S/R)-16-Chloro-7,11-dihydroxy-3-(1-methyl-2-(2-methyl-4-thiazolyl)ethenyl)-8,8,10,12-tetramethyl-4,17-dioxabicyclo[14.1.0]heptadecane-5,9-dione

(4S,7R,8 S,9S,13E/Z,16S(E))-4,8-Dihydroxy-13-cyano-16-(1-methyl-2-(2-methyl-4-thiazolyl)ethenyl)-1-oxa-5,5,7,9-tetraamethyl-cyclohexadec-13-ene-2,6-dione

(1S/R,3S(E),7S,10R,11 S,12S,16S/R)-16-Cyano-7,11-dihydroxy-3-(1-methyl-2-(2-methyl-4-thiazolyl)ethenyl)-8,8,10,12-tetramethyl-4,17-dioxabicyclo[14.1.0]heptadecane-5,9-dione

(4S,7R,8S,9S,13E/Z,16S(E))-4,8-Dihydroxy-7-ethyl-13-fluoro-16-(1-methyl-2-(2-methyl-4-thiazolyl)ethenyl)-1-oxa-5,5,9-trimethyl-cyclohexadec-13-ene-2,6-dione

(1S/R,3S(E),7S,10R,11S,12S,16S/R)-16-Fluoro-10-ethyl-7,11-dihydroxy-3-(1-methyl-2-(2-methyl-4-thiazolyl)ethenyl)-8,8,12-trimethyl-4,17-dioxabicyclo[14.1.0]heptadecane-5,9-dione

(4S,7R,8S,9S,13E/Z,16S(E))-4,8-Dihydroxy-7-allyl-13-chloro-16-(1-methyl-2-(2-methyl-4-thiazolyl)ethenyl)-1-oxa-5,5,9-trimethyl-cyclohexadec-13-ene-2,6-dione

(1S/R,3S(E),7S,10R,11 S,12S,16S/R)-16-Chloro-10-allyl-7,11-dihydroxy-3-(1-methyl-2-(2-methyl-4-thiazolyl)ethenyl)-8,8,12-trimethyl-4,17-dioxabicyclo [14.1.0]heptadecane-5,9-dione

(4S,7R,8S,9S,13E/Z,16S(E))-4,8-Dihydroxy-13-chloro-16-(1-methyl-2-(2-pyridyl)ethenyl)-1-oxa-5,5,7,9-tetraamethyl-cyclohexadec-13-ene-2,6-dione

(1S/R,3S(E),7S,10R,11 S,12S,16S/R)-16-Chloro-7,11-dihydroxy-3-(1-methyl-2-(2-pyridyl)ethenyl)-8,8,10,12-tetramethyl-4,17-dioxabicyclo[14.1.0]heptadecane-5,9-dione

(4S,7R,8S,9S,13E/Z,16S(Z))-4,8-Dihydroxy-13-chloro-16-(1-fluoro-2-(2-pyridyl)ethenyl)-1-oxa-5,5,7,9-tetraamethyl-cyclohexadec-13-ene-2,6-dione

(1S/R,3S(Z),7S,10R,11 S,12S,16S/R)-16-Chloro-7,11-dihydroxy-3-(1-fluoro-2-(2-pyridyl)ethenyl)-8,8,10,12-tetramethyl-4,17-dioxabicyclo[14.1.0]heptadecane-5,9-dione

(4S,7R,8S,9S,13E/Z,16S(Z))-4,8-Dihydroxy-7-ethyl-13-chloro-16-(1-chloro-2-(2-pyridyl)ethenyl)-1-oxa-5,5,9-trimethyl-cyclohexadec-13-ene-2,6-dione

(1S/R,3S(Z),7S,10R,11 S,12S,16S/R)-16-Chloro-7,11-dihydroxy-7-ethyl-3-(1-chloro-2-(2-pyridyl)ethenyl)-8,8,12-trimethyl-4,17-dioxabicyclo[14.1.0]heptadecane-5,9-dione

(4S,7R,8S,9S,13E/Z,16S(Z))-4,8-Dihydroxy-13-chloro-16-(1-chloro-2-(2-methyl-4-thiazolyl)ethenyl)-1-oxa-5,5,7,9-tetraamethyl-cyclohexadec-13-ene-2,6-dione

(1S/R,3S(Z),7S,10R,11 S,12S,16S/R)-16-Chloro-7,11-dihydroxy-3-(1-chloro-2-(2-methyl-4-thiazolyl)ethenyl)-8,8,10,12-tetramethyl-4,17-dioxabicyclo[14.1.0]heptadecane-5,9-dione

(4S,7R,8S,9S,13E/Z,16S(Z))-4,8-Dihydroxy-13-chloro-16-(1-fluoro-2-(2-methyl-4-thiazolyl)ethenyl)-5,5-trimethylen-1-oxa-7,9-dimethyl-cyclohexadec-13-ene-2,6-dione

(1S/R,3S(Z),7S,10R,11S,12S,16S/R)-16-Chloro-7,11-dihydroxy-3-(1-fluoro-2-(2-methyl-4-thiazolyl)ethenyl)-8,8-trimethylene-10,12-dimethyl-4,17-dioxabicyclo[14.1.0]heptadecane-5,9-dione

(4S,7R,8S,9S,13E/Z,16S(Z))-4,8-Dihydroxy-13-chloro-16-(1-chloro-2-(2-methyl-4-thiazolyl)ethenyl)-5,5-trimethylen-1-oxa-7,9-dimethyl-cyclohexadec-13-ene-2,6-dione

(1S/R,3S(Z),7S,10R,11 S,12S,16S/R)-16-Chloro-7,11-dihydroxy-3-(1-chloro-2-(2-methyl-4-thiazolyl)ethenyl)-8,8-trimethylene-10,12-dimethyl-4,17-dioxabicyclo [14.1.0]heptadecane-5,9-dione

(4S,7R,8S,9S,13E/Z,16S)-4,8-Dihydroxy-13-fluoro-16-(2-methyl-5-benzothiazolyl)-1-oxa-5,5,7,9-tetramethyl-cyclohexadec-13-ene-2,6-dione

(1S/R,3S,7S,10R,11 S,12S,16S/R)— 16-Fluoro-7,11-dihydroxy-3-(2-methyl-5-benzothiazolyl)-8,8,10,12-tetramethyl-4,17-dioxabicyclo[14.1.0]heptadecane-5,9-dione

(4S,7R,8S,9S,13E/Z,16S)-4,8-Dihydroxy-13-chloro-16-(2-methyl-5-benzothiazolyl)-1-oxa-5,5,7,9-tetraamethyl-cyclohexadec-13-ene-2,6-dione

(1S/R,3S,7S,10R,11 S,12S,16S/R)-16-Chloro-7,11-dihydroxy-3-(2-methyl-5-benzothiazolyl)-8,8,10,12-tetramethyl-4,17-dioxabicyclo[14.1.0]heptadecane-5,9-dione

(4S,7R,8S,9S,13E/Z,16S)-4,8-Dihydroxy-7-ethyl-13-chloro-16-(2-methyl-5-benzothiazolyl)-1-oxa-5,5,9-trimethyl-cyclohexadec-13-ene-2,6-dione

(1S/R,3S,7S,10R,11 S,12S,16S/R)-16-Chloro-7,11-dihydroxy-10-ethyl-3-(2-methyl-5-benzothiazolyl)-8,8,12-trimethyl-4,17-dioxabicyclo [14.1.0]heptadecane-5,9-dione

(4S,7R,8S,9S,13E/Z,16S)-4,8-Dihydroxy-7-allyl-13-chloro-16-(2-methyl-5-benzothiazolyl)-1-oxa-5,5,9-trimethyl-cyclohexadec-13-ene-2,6-dione

(1S/R,3S,7S,10R,11 S,12S,16S/R)-16-Chloro-7,11-dihydroxy-10-allyl-3-(2-methyl-5-benzothiazolyl)-8,8,12-trimethyl-4,17-dioxabicyclo[14.1.0]heptadecane-5,9-dione

(4S,7R,8S,9S,13E/Z,16S(E))-4,8-Dihydroxy-13-fluoro-16-(1-methyl-2-(2-methyl-4-thiazolyl)ethenyl)-1-aza-11-oxa-5,5,7,9-tetramethyl-cyclohexadec-13-ene-2,6-dione

(1S/R,3S(E),7S,10R,11 S,12S,16S/R)— 16-Fluoro-7,11-dihydroxy-3-(1-methyl-2-(2-methyl-4-thiazolyl)ethenyl)-8,8,10,12-tetramethyl-4-aza-14,17-dioxabicyclo[14.1.0]heptadecane-5,9-dione

(4S,7R,8 S,9S,13E/Z,16S(E))-4,8-Dihydroxy-13-chloro-16-(1-methyl-2-(2-methyl-4-thiazolyl)ethenyl)-1-aza-11-oxa-5,5,7,9-tetraamethyl-cyclohexadec-13-ene-2,6-dione

(1S/R,3 S(E),7S,10R,11 S,12S,16S/R)-16-Chloro-7,11-dihydroxy-3-(1-methyl-2-(2-methyl-4-thiazolyl)ethenyl)-8,8,10,12-tetramethyl-4-aza-14,17-dioxabicyclo[14.1.0]heptadecane-5,9-dione

(4S,7R,8S,9S,13E/Z,16S(E))-4,8-Dihydroxy-13-cyano-16-(1-methyl-2-(2-methyl-4-thiazolyl)ethenyl)-1-aza-11-oxa-5,5,7,9-tetraamethyl-cyclohexadec-13-ene-2,6-dione

(1S/R,3S(E),7S,10R,11 S,12S,16S/R)-16-Cyano-7,11-dihydroxy-3-(1-methyl-2-(2-methyl-4-thiazolyl)ethenyl)-8,8,10,12-tetramethyl-4-aza-14,17-dioxabicyclo[14.1.0]heptadecane-5,9-dione

(4S,7R,8S,9S,13E/Z,16S(E))-4,8-Dihydroxy-13-cyano-16-(1-methyl-2-(2-methyl-4-thiazolyl)ethenyl)-1-aza-11-oxa-5,5,7,9-tetraamethyl-cyclohexadec-13-ene-2,6-dione

(1S/R,3S(E),7S,10R,11 S,12S,16S/R)-16-Cyano-7,11-dihydroxy-3-(1-methyl-2-(2-methyl-4-thiazolyl)ethenyl)-8,8,10,12-tetramethyl-4-aza-14,17-dioxabicyclo [14.1.0]heptadecane-5,9-dione

(4S,7R,8S,9S,13E/Z,16S(E))-4,8-Dihydroxy-7-ethyl-13-fluoro-16-(1-methyl-2-(2-methyl-4-thiazolyl)ethenyl)-1-aza-11-oxa-5,5,9-trimethyl-cyclohexadec-13-ene-2,6-dione

(1S/R,3S(E),7S,10R,11 S,12S,16S/R)-16-Fluoro-10-ethyl-7,11-dihydroxy-3-(1-methyl-2-(2-methyl-4-thiazolyl)ethenyl)-8,8,12-trimethyl-4-aza-14,17-dioxabicyclo [14.1.0]heptadecane-5,9-dione

(4S,7R,8S,9S,13E/Z,16S(E))-4,8-Dihydroxy-7-allyl-13-chloro-16-(1-methyl-2-(2-methyl-4-thiazolyl)ethenyl)-1-aza-11-oxa-5,5,9-trimethyl-cyclohexadec-13-ene-2,6-dione

(1S/R,3S(E),7S,10R,11 S,12S,16S/R)-16-Chloro-10-allyl-7,11-dihydroxy-3-(1-methyl-2-(2-methyl-4-thiazolyl)ethenyl)-8,8,12-trimethyl-4-aza-14,17-dioxabicyclo[14.1.0]heptadecane-5,9-dione

(4S,7R,8S,9S,13E/Z,16S(E))-4,8-Dihydroxy-13-chloro-16-(1-methyl-2-(2-pyridyl)ethenyl)-1-aza-11-oxa-5,5,7,9-tetraamethyl-cyclohexadec-13-ene-2,6-dione

(1S/R,3S(E),7S,10R,11 S,12S,16S/R)-16-Chloro-7,11-dihydroxy-3-(1-methyl-2-(2-pyridyl)ethenyl)-8,8,10,12-tetramethyl-4-aza-14,17-dioxabicyclo[14.1.0]heptadecane-5,9-dione

(4S,7R,8S,9S,13E/Z,16S(Z))-4,8-Dihydroxy-13-chloro-16-(1-fluoro-2-(2-methyl-4-thiazolyl)ethenyl)-1-aza-11-oxa-5,5,7,9-tetraamethyl-cyclohexadec-13-ene-2,6-dione

(1S/R,3S(Z),7S,10R,11 S,12S,16S/R)-16-Chloro-7,11-dihydroxy-3-(1-fluoro-2-(2-methyl-4-thiazolyl)ethenyl)-8,8,10,12-tetramethyl-4-aza-14,17-dioxabicyclo [14.1.0]heptadecane-5,9-dione

(4S,7R,8S,9S,13E/Z,16S(Z))-4,8-Dihydroxy-13-chloro-16-(1-chloro-2-(2-methyl-4-thiazolyl)ethenyl)-1-aza-11-oxa-5,5,7,9-tetraamethyl-cyclohexadec-13-ene-2,6-dione

(1S/R,3 S (Z),7S,10R,11 S,12S,16S/R)-16-Chloro-7,11-dihydroxy-3-(1-chloro-2-(2-methyl-4-thiazolyl)ethenyl)-8,8,10,12-tetramethyl-4-aza-14,17-dioxabicyclo[14.1.0]heptadecane-5,9-dione

(4S,7R,8S,9S,13E/Z,16S(Z))-4,8-Dihydroxy-13-chloro-16-(1-fluoro-2-(2-methyl-4-thiazolyl)ethenyl)-5,5-trimethylen-1-aza-11-oxa-7,9-dimethyl-cyclohexadec-13-ene-2,6-dione

(1S/R,3 S(Z),7S,10R,11 S,12S,16S/R)-16-Chloro-7,11-dihydroxy-3-(1-fluoro-2-(2-methyl-4-thiazolyl)ethenyl)-8,8-trimethylene-10,12-dimethyl-4-aza-14,17-dioxabicyclo[14.1.0]-heptadecane-5,9-dione

(4S,7R,8S,9S,13E/Z,16S(Z))-4,8-Dihydroxy-13-chloro-16-(1-chloro-2-(2-methyl-4-thiazolyl)ethenyl)-5,5-trimethylen-1-aza-11-oxa-7,9-dimethyl-cyclohexadec-13-ene-2,6-dione

(1S/R,3S(Z),7S,10R,11 S,12S,16S/R)-16-Chloro-7,11-dihydroxy-3-(1-chloro-2-(2-methyl-4-thiazolyl)ethenyl)-8,8-trimethylene-10,12-dimethyl-4-aza-14,17-dioxabicyclo[14.1.0]-heptadecane-5,9-dione

(4S,7R,8S,9S,13E/Z,16S(Z))-4,8-Dihydroxy-13-chloro-16-(1-fluoro-2-(2-methyl-4-oxazolyl)ethenyl)-1-aza-11-oxa-5,5,7,9-tetraamethyl-cyclohexadec-13-ene-2,6-dione

(1S/R,3S(Z),7S,10R,11 S,12S,16S/R)-16-Chloro-7,11-dihydroxy-3-(1-fluoro-2-(2-methyl-4-oxazolyl)ethenyl)-8,8,10,12-tetramethyl-4-aza-14,17-dioxabicyclo[14.1.0]heptadecane-5,9-dione

(4S,7R,8S,9S,13E/Z,16S(Z))-4,8-Dihydroxy-13-chloro-16-(1-fluoro-2-(2-pyridyl)ethenyl)-1-aza-11-oxa-5,5,7,9-tetraamethyl-cyclohexadec-13-ene-2,6-dione

(1S/R,3S(Z),7S,10R,11 S,12S,16S/R)-16-Chloro-7,11-dihydroxy-3-(1-fluoro-2-(2-pyridyl)ethenyl)-8,8,10,12-tetramethyl-4-aza-14,17-dioxabicyclo[14.1.0]heptadecane-5,9-dione

(4S,7R,8S,9S,13E/Z,16S(Z))-4,8-Dihydroxy-7-ethyl-13-chloro-16-(1-chloro-2-(2-pyridyl)ethenyl)-1-aza-11-oxa-5,5,9-trimethyl-cyclohexadec-13-ene-2,6-dione

(1S/R,3 S(Z),7S,10R,11 S,12S,16S/R)-16-Chloro-7,11-dihydroxy-7-ethyl-3-(1-chloro-2-(2-pyridyl)ethenyl)-8,8,12-trimethyl-4-aza-14,17-dioxabicyclo[14.1.0]heptadecane-5,9-dione

(4S,7R,8S,9S,13E/Z,16S)-4,8-Dihydroxy-13-fluoro-16-(2-methyl-5-benzothiazolyl)-1-aza-11-oxa-5,5,7,9-tetramethyl-cyclohexadec-13-ene-2,6-dione

(1S/R,3S,7S,10R,11S,12S,16S/R)-16-Fluoro-7,11-dihydroxy-3-(2-methyl-5-benzothiazolyl)-8,8,10,12-tetramethyl-4-aza-14,17-dioxabicyclo [14.1.0]heptadecane-5,9-dione

(4S,7R,8S,9S,13E/Z,16S)-4,8-Dihydroxy-13-chloro-16-(2-methyl-5-benzothiazolyl)-1-aza-11-oxa-5,5,7,9-tetraamethyl-cyclohexadec-13-ene-2,6-dione (1S/R,3S,7S,10R,11S,12S,16S/R)-16-Chloro-7,11-dihydroxy-3-(2-methyl-5-benzothiazolyl)-8,8,10,12-tetramethyl-4-aza-14,17-dioxabicyclo[14.1.0]heptadecane-5,9-dione

(4S,7R,8S,9S,13E/Z,16S)-4,8-Dihydroxy-13-cyano-16-(2-methyl-5-benzothiazolyl)-1-aza-11-oxa-5,5,7,9-tetraamethyl-cyclohexadec-13-ene-2,6-dione

(1S/R,3S,7S,10R,11S,12S,16S/R)-16-Cyano-7,11-dihydroxy-3-(2-methyl-5-benzothiazolyl)-8,8,10,12-tetramethyl-4-aza-14,17-dioxabicyclo[14.1.0]heptadecane-5,9-dione

(4S,7R,8S,9S,13E/Z,16S)-4,8-Dihydroxy-7-ethyl-13-chloro-16-(2-methyl-5-benzothiazolyl)-1-aza-11-oxa-5,5,9-trimethyl-cyclohexadec-13-ene-2,6-dione

(1S/R,3S,7S,10R,11S,12S,16S/R)-16-Chloro-7,11-dihydroxy-10-ethyl-3-(2-methyl-5-benzothiazolyl)-8,8,12-trimethyl-4-aza-14,17-dioxabicyclo[14.1.0]heptadecane-5,9-dione

(4S,7R,8S,9S,13E/Z,16S)-4,8-Dihydroxy-7-allyl-13-chloro-16-(2-methyl-5-benzothiazolyl)-1-aza-11-oxa-5,5,9-trimethyl-cyclohexadec-13-ene-2,6-dione

(1S/R,3 S,7S,10R,11S,12S,16S/R)-16-Chloro-7,11-dihydroxy-10-allyl-3-(2-methyl-5-benzothiazolyl)-8,8,12-trimethyl-4-aza-14,17-dioxabicyclo[14.1.0]heptadecane-5,9-dione

(4S,7R,8S,9S,13E/Z,16S)-4,8-Dihydroxy-13-fluoro-16-(2-methyl-5-benzoxazolyl)-1-aza-11-oxa-5,5,7,9-tetramethyl-cyclohexadec-13-ene-2,6-dione

(1S/R,3S,7S,10R,11S,12S,16S/R)-16-Fluoro-7,11-dihydroxy-3-(2-methyl-5-benzoxazolyl)-8,8,10,12-tetramethyl-4-aza-14,17-dioxabicyclo[14.1.0]heptadecane-5,9-dione

(4S,7R,8S,9S,13E/Z,16S)-4,8-Dihydroxy-13-chloro-16-(2-methyl-5-benzoxazolyl)-1-aza-11-oxa-5,5,7,9-tetraamethyl-cyclohexadec-13-ene-2,6-dione

(1S/R,3S,7S,10R,11S,12S,16S/R)-16-Chloro-7,11-dihydroxy-3-(2-methyl-5-benzoxazolyl)-8,8,10,12-tetramethyl-4-aza-14,17-dioxabicyclo[14.1.0]heptadecane-5,9-dione

(4S,7R,8S,9S,13E/Z,16S(E))-4,8-Dihydroxy-13-chloro-16-(1-methyl-2-(2-methyl-4-thiazolyl)ethenyl)-1-aza-5,5,7,9-tetraamethyl-cyclohexadec-13-ene-2,6-dione

(1S/R,3S(E),7S,10R,11S,12S,16S/R)-16-Chloro-7,11-dihydroxy-3-(1-methyl-2-(2-methyl-4-thiazolyl)ethenyl)-8,8,10,12-tetramethyl-4-aza-17-oxabicyclo[14.1.0]heptadecane-5,9-dione

(4S,7R,8S,9S,13E/Z,16S(E))-4,8-Dihydroxy-7-ethyl-13-fluoro-16-(1-methyl-2-(2-methyl-4-thiazolyl)ethenyl)-1-aza-5,5,9-trimethyl-cyclohexadec-13-ene-2,6-dione

(1S/R,3S(E),7S,10R,11S,12S,16S/R)-16-Fluoro-10-ethyl-7,11-dihydroxy-3-(1-methyl-2-(2-methyl-4-thiazolyl)ethenyl)-8,8,12-trimethyl-4-aza-17-oxabicyclo[14.1.0]heptadecane-5,9-dione

(4S,7R,8S,9S,13E/Z,16S(E))-4,8-Dihydroxy-7-allyl-13-chloro-16-(1-methyl-2-(2-methyl-4-thiazolyl)ethenyl)-1-aza-5,5,9-trimethyl-cyclohexadec-13-ene-2,6-dione

(1S/R,3S(E),7S,10R,11S,12S,16S/R)-16-Chloro-10-allyl-7,11-dihydroxy-3-(1-methyl-2-(2-methyl-4-thiazolyl)ethenyl)-8,8,12-trimethyl-4-aza-17-oxabicyclo[14.1.0]heptadecane-5,9-dione

(4S,7R,8S,9S,13E/Z,16S(E))-4,8-Dihydroxy-13-chloro-16-(1-methyl-2-(2-pyridyl)ethenyl)-1-aza-5,5,7,9-tetraamethyl-cyclohexadec-13-ene-2,6-dione

(1S/R,3S(E),7S,10R,11S,12S,16S/R)-16-Chloro-7,11-dihydroxy-3-(1-methyl-2-(2-pyridyl)ethenyl)-8,8,10,12-tetramethyl-4-aza-17-oxabicyclo[14.1.0]heptadecane-5,9-dione

(4S,7R,8S,9S,13E/Z,16S(Z))-4,8-Dihydroxy-13-chloro-16-(1-fluoro-2-(2-pyridyl)ethenyl)-1-aza-5,5,7,9-tetraamethyl-cyclohexadec-13-ene-2,6-dione

(1S/R,3S(Z),7S,10R,11 S,12S,16S/R)-16-Chloro-7,11-dihydroxy-3-(1-fluoro-2-(2-pyridyl)ethenyl)-8,8,10,12-tetramethyl-4-aza-17-oxabicyclo [14.1.0]heptadecane-5,9-dione

(4S,7R,8S,9S,13E/Z,16S(Z))-4,8-Dihydroxy-7-ethyl-13-chloro-16-(1-chloro-2-(2-pyridyl)ethenyl)-1-aza-5,5,9-trimethyl-cyclohexadec-13-ene-2,6-dione

(1S/R,3S(Z),7S,10R,11 S,12S,16S/R)-16-Chloro-7,11-dihydroxy-7-ethyl-3-(1-chloro-2-(2-pyridyl)ethenyl)-8,8,12-trimethyl-4-aza-17-oxabicyclo[14.1.0]heptadecane-5,9-dione

(4S,7R,8S,9S,13E/Z,16S(Z))-4,8-Dihydroxy-13-chloro-16-(1-chloro-2-(2-methyl-4-thiazolyl)ethenyl)-1-aza-5,5,7,9-tetraamethyl-cyclohexadec-13-ene-2,6-dione

(1S/R,3S(Z),7S,10R,11 S,12S,16S/R)-16-Chloro-7,11-dihydroxy-3-(1-chloro-2-(2-methyl-4-thiazolyl)ethenyl)-8,8,10,12-tetramethyl-4-aza-17-oxabicyclo[14.1.0]heptadecane-5,9-dione

(4S,7R,8S,9S,13E/Z,16S(Z))-4,8-Dihydroxy-13-chloro-16-(1-fluoro-2-(2-methyl-4-thiazolyl)ethenyl)-5,5-trimethylen-1-aza-7,9-dimethyl-cyclohexadec-13-ene-2,6-dione

(1S/R,3S(Z),7S,10R,11S,12S,16S/R)-16-Chloro-7,11-dihydroxy-3-(1-fluoro-2-(2-methyl-4-thiazolyl)ethenyl)-8,8-trimethylene-10,12-dimethyl-4-aza-17-oxabicyclo[14.1.0]heptadecane-5,9-dione

(4S,7R,8 S,9S,13E/Z,16S(Z))-4,8-Dihydroxy-13-chloro-16-(1-chloro-2-(2-methyl-4-thiazolyl)ethenyl)-5,5-trimethylen-1-aza-7,9-dimethyl-cyclohexadec-13-ene-2,6-dione

(1S/R,3S(Z),7S,10R,11 S,12S,16S/R)-16-Chloro-7,11-dihydroxy-3-(1-chloro-2-(2-methyl-4-thiazolyl)ethenyl)-8,8-trimethylene-10,12-dimethyl-4-aza-17-oxabicyclo[14.1.0]heptadecane-5,9-dione

(4S,7R,8S,9S,13E/Z,16S)-4,8-Dihydroxy-13-fluoro-16-(2-methyl-5-benzothiazolyl)-1-aza-5,5,7,9-tetramethyl-cyclohexadec-13-ene-2,6-dione

(1S/R,3S,7S,10R,11 S,12S,16S/R)-16-Fluoro-7,11-dihydroxy-3-(2-methyl-5-benzothiazolyl)-8,8,10,12-tetramethyl-4-aza-17-oxabicyclo[14.1.0]heptadecane-5,9-dione

(4S,7R,8S,9S,13E/Z,16S)-4,8-Dihydroxy-13-chloro-16-(2-methyl-5-benzothiazolyl)-1-aza-5,5,7,9-tetraamethyl-cyclohexadec-13-ene-2,6-dione

(1S/R,3S,7S,10R,11S,12S,16S/R)-16-Chloro-7,11-dihydroxy-3-(2-methyl-5-benzothiazolyl)-8,8,10,12-tetramethyl-4-aza-17-oxabicyclo[14.1.0]heptadecane-5,9-dione

(4S,7R,8S,9S,13E/Z,16S)-4,8-Dihydroxy-13-cyano-16-(2-methyl-5-benzothiazolyl)-1-aza-5,5,7,9-tetraamethyl-cyclohexadec-13-ene-2,6-dione

(1S/R,3S,7S,10R,11 S,12S,16S/R)-16-Cyano-7,11-dihydroxy-3-(2-methyl-5-benzothiazolyl)-8,8, 10,12-tetramethyl-4-aza-17-oxabicyclo[14.1.0]heptadecane-5,9-dione

(4S,7R,8S,9S,13E/Z,16S)-4,8-Dihydroxy-7-ethyl-13-chloro-16-(2-methyl-5-benzothiazolyl)-1-aza-5,5 ,9-trimethyl-cyclohexadec-13-ene-2,6-dione

(1S/R,3 S,7S,10R,11S,12S,16S/R)-16-Chloro-7,11-dihydroxy-10-ethyl-3-(2-methyl-5-benzothiazolyl)-8,8, 12-trimethyl-4-aza-17-oxabicyclo[14.1.0]heptadecane-5,9-dione

(4S,7R,8S,9S,13E/Z,16S)-4,8-Dihydroxy-7-allyl-13-chloro-16-(2-methyl-5-benzothiazolyl)-1-aza-5,5,9-trimethyl-cyclohexadec-13-ene-2,6-dione

(1S/R,3S,7S,10R,11 S,12S,16S/R)-16-Chloro-7,1-dihydroxy-10-allyl-3-(2-methyl-5-benzothiazolyl)-8,8, 12-trimethyl-4-aza-17-oxabicyclo[14.1.0]heptadecane-5,9-dione

Representation of Partial Fragments A:

The partial fragments (synthesis components) of general formula A can easily be from

a) a pantolactone of general formula IIa

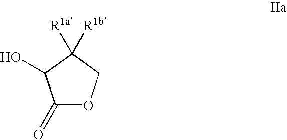

in which

R^{1a′}, R^{1b′} in each case stand for a methyl group, or

b) a malonic acid dialkyl ester of general formula XXVIII

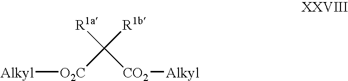

in which

R^{1a′}, R^{1b′} have the meaning that is indicated in general formula A, and alkyl, independently of one another, mean a C₁-C₂₀-alkyl-, C₃-C₁₀-cycloalkyl- or C₄-C₂₀-alkylcycloalkyl radical.

Partial fragments A, in which R^{1a}′=R^{1b′}=methyl, can be efficiently produced from inexpensive pantolactone with an optical purity of >98%.

The synthesis is described in Diagram 1 below in the example of D-(−)-pantolactone. From L-(+)-pantolactone are obtained the corresponding enantiomeric compounds ent-A-II to ent-A-XIV in A-II to A-XIV, and from racemic DL-pantolactone are obtained the corresponding racemic compounds rac-A-II to rac-A-XIV:

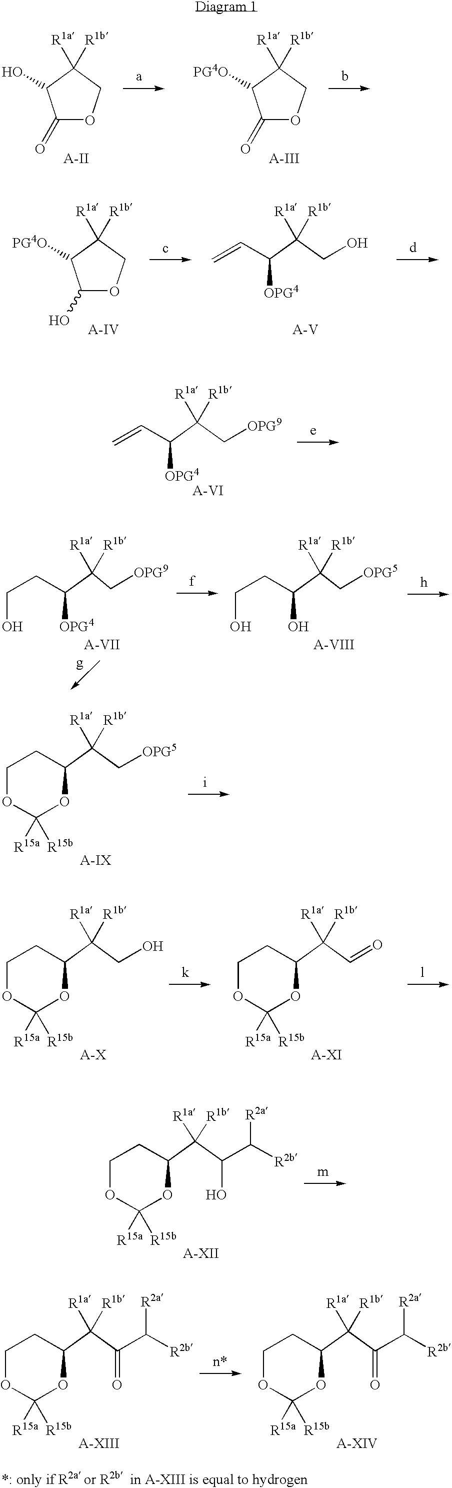

Step a (A-II→A-III):

The free hydroxy group of pantolactone (A-II) is protected according to the methods that are known to one skilled in the art. As protective group PG⁴, the protective groups that are known to one skilled in the art, such as, e.g., methoxymethyl, methoxyethyl, ethoxyethyl, tetrahydropyranyl, tetrahydrofuranyl, trimethylsilyl, triethylsilyl, tert-butyldimethylsilyl, tert-butyldiphenylsilyl, tribenzylsilyl, triisopropylsilyl, benzyl, para-nitrobenzyl, para-methoxybenzyl, trityl, dimethoxytrityl, formyl, acetyl, propionyl, isopropionyl, pivalyl, butyryl or benzoyl radicals, are suitable.

A survey is found in, e.g., “Protective Groups in Organic Synthesis” Theodora W. Green, John Wiley and Sons).

Preferred are those protective groups that can be cleaved under acidic reaction conditions, such as, e.g., methoxymethyl, tetrahydropyranyl, tetrahydrofuranyl, and trimethylsilyl radicals.

Especially preferred is the tetrahydropyranyl radical.

Step b (A-III→A-IV):

Protected lactone A-III is reduced to lactol A-IV. As a reducing agent, aluminum hydrides that are modified in their reactivity, such as, e.g., diisobutylaluminum hydride, are suitable. The reaction is carried out in an inert solvent such as, e.g., toluene, preferably at low temperatures.

Step c (A-IV→A-V):

Lactol A-IV is opened up to form hydroxyolefin A-V while expanding by one C atom. For this purpose, the methods that are known to one skilled in the art, such as, e.g., olefination according to Tebbe, the Wittig or Wittig/Homer reaction, the addition of an organometallic compound with dehydration, are suitable. Preferred is the Wittig reaction with use of methyltriarylphosphonium halides such as, e.g., methyltriphenylphosphonium bromide with strong bases, such as, e.g., n-butyllithium, potassium-tert-butanolate, sodium ethanolate, sodium hexamethyldisilazane; as a base, n-butyllithium is preferred.

Step d (A-V→A-VI):

The free hydroxy group in A-V is protected according to the methods that are known to one skilled in the art. As protective group PG⁵, the protective groups that are known to one skilled in the art, as were already mentioned above for PG⁴ in step a (A-II→A-III), are suitable.

Preferred are those protective groups that can be cleaved under the action of fluoride, such as, e.g., the trimethylsilyl, tert-butyldimethylsilyl, tert-butyldiphenylsilyl, tribenzylsilyl, or triisopropylsilyl radical.

Especially preferred is the tert-butyldimethylsilyl, the triisopropylsilyl and the tert-butyldiphenylsilyl radical.

Step e (A-VI→A-VII):

Water is added to the double bond in A-VI in an anti-Markovnikov orientation. For this purpose, the processes that are known to one skilled in the art, such as, e.g., reaction with boranes, their subsequent oxidation to the corresponding boric acid esters and their saponification are suitable. As boranes, e.g., the borane-tetrahydrofuran complex, the borane-dimethyl sulfide complex, 9-borabicyclo[3.3.1]nonane in an inert solvent such as, for example, tetrahydrofuran or diethyl ether, are preferred. As oxidizing agents, preferably hydrogen peroxide is used; for saponification of the boron esters, preferably alkali hydroxides, such as, e.g., sodium hydroxide, are used.

Step f (A-VI→A-VII):

Protective group PG⁴ that is introduced under step a) is now cleaved according to the processes that are known to one skilled in the art. If this is a protective group that can be cleaved acidically, then cleavage can be accomplished with dilute mineral acids in aqueous-alcoholic solutions and with the aid of catalytic quantities of acids, such as, e.g., para-toluenesulfonic acid, para-toluenesulfonic acid-pyridinium salt, camphorsulfonic acid in alcoholic solutions, preferably in ethanol or isopropanol.

Step g (A-VII→A-IX):

Common protection of the two alcohol functions of the mono-protected 1,3-diol in A-VU is possible under acid catalysis by direct ketalization with a carbonyl compound of general formula R^{15a}—CO—R^{15b} or by reketalization with a ketal of general formulas R^{15a}—C(OC₂H₅)₂—R^{15b}, R^{15a}—C(OC₂H₄)₂—R^{15b}, and R^{15a}—C(OCH₂C(CH₃)₂CH₂O)—R^{15b}, in which in each case R^{15a} and R^{15b} have the above-indicated meanings. As acids, the acids already mentioned under step f) are suitable; the use of para-toluenesulfonic acid optionally with the addition of copper(II) or cobalt(II) salts, such as, e.g., copper(II) sulfate, is preferred.

Step h (A-VIII→A-IX):

Protection of the two alcohol functions of 1,3-diol in A-VIII is possible under acid catalysis by direct ketalization with a carbonyl compound of general formula R^{15a}—CO—R^{15b}, or by reketalization with a ketal of general formulas R^{15a}—C(OC₂H₅)₂—R^{15b}, R^{15a}—C(OC₂H₄)₂—R^{15b}, R^{15a}—C(OCH₂C(CH₃)₂CH₂O)—R^{15b}, in which in each case R^{15a} and R^{15b} have the above-indicated meanings. Reketalization preferably with 2,2-dimethoxypropane is preferred. As acids, the acids already mentioned under step f) are suitable, and the use of camphorsulfonic acid is preferred.

Step i (A-IX→A-X):

Protective group PG⁵ introduced under step d) is now cleaved according to the process that is known to one skilled in the art. If this is a silyl ether, thus suitable for the cleavage are the reaction with fluorides, such as, for example, tetrabutylammonium fluoride, the hydrogen fluoride-pyridine complex, potassium fluoride or the use of dilute mineral acids, the use of catalytic quantities of acids, such as, e.g., para-toluenesulfonic acid, para-toluenesulfonic acid-pyridinium salt, and camphorsulfonic acid in alcoholic solutions, preferably in ethanol or isopropanol.

Step k (A-X→A-XI):

The oxidation of the primary alcohol in A-X to aldehyde is carried out according to the methods that are known to one skilled in the art. For example, oxidation with pyridinium chlorochromate, pyridinium dichromate, chromium trioxide-pyridine complex, oxidation according to Swem or related methods, e.g., with use of oxalyl chloride in dimethyl sulfoxide, the use of Dess-Martin periodinane, the use of nitrogen oxides, such as, e.g., N-methyl-morpholino-N-oxide in the presence of suitable catalysts, such as, e.g., tetrapropylammonium perruthenate in inert solvents, can be mentioned. Preferred is the oxidation according to Swem, with the SO₃-pyridine complex, as well as with N-methyl-morpholino-N-oxide using tetrapropylammonium perruthenate.

Step 1 (A-XI→A-XII):

The reaction of aldehydes A-XI to form alcohols of formula A-XII is carried out with organometallic compounds of general formula M-CHR^{2a′}R^{2b′}, in which M stands for an alkali metal, preferably lithium, or a divalent metal MX, in which X represents a halogen, and radicals R^{2a′} and R^{2b′} in each case have the above-mentioned meanings. As a divalent metal, magnesium and zinc are preferred; as halogen X, chlorine, bromine and iodine are preferred.

Step m (A-XII→A-XIII):

Oxidation of the secondary alcohol in A-XII to ketone A-XIII is carried out according to the conditions that are mentioned under step k). Oxidation with N-methyl-morpholino-N-oxide with use of tetrapropylammonium perruthenate is preferred.

Step n (A-III→A-XIV):

If R^{2a′}in A-XIII is equal to hydrogen, the possibility exists of introducing for this purpose a second radical R^{2a′}, which has the above-mentioned meanings, excluding hydrogen. For this purpose, ketone in A-XIII is introduced into the enolate with use of strong bases, such as, e.g., lithium diisopropylamide, and reacted with a compound of general formula X-R^{2a′}, in which X represents a halogen. As halogen X, chlorine, bromine and iodine are preferred.

The previously described path can also be used to synthesize C1-C6-epothilone components, which on C-1 contain a carboxylic acid or their esters (R¹³═CO₂R^{13b} in A).

The synthesis of component A-XXII is described in Diagram 2 below in the example of intermediate stage A-V that is derived from D-(−)-pantolactone. The corresponding enantiomer compounds ent-A-V to ent-A-XXVII in A-V to A-XXVII are obtained from L-(+)-pantolactone, and the corresponding racemic compounds rac-A-V to rac-A-XXVII are obtained from racemic DL-pantolactone:

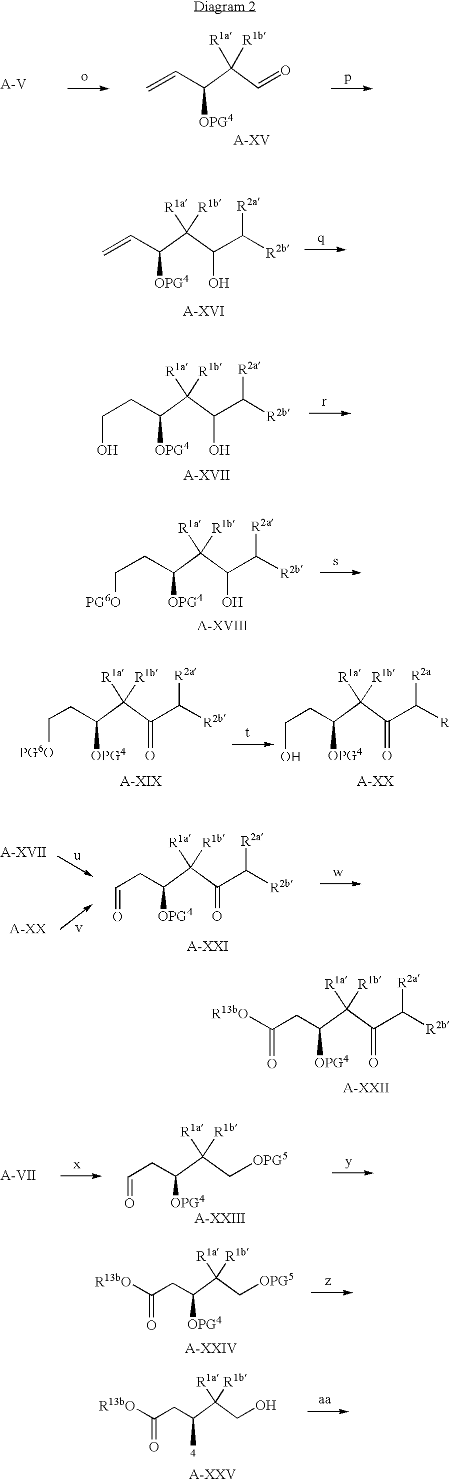

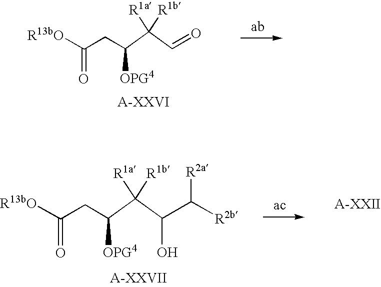

Step o (A-V→A-XV):

Oxidation of the primary alcohol in A-V to aldehyde A-XV is carried out according to the conditions that are mentioned under step k). The oxidation process according to Swem is preferred.

Step p (A-XV→A-XVI):

The reaction of aldehydes A-XV to form alcohols of formula A-XVI is carried out with organometallic compounds of general formula M-CHR^{2a′}R^{2b′}, in which M stands for an alkali metal, preferably lithium, or a divalent metal MX, in which X represents a halogen, and radicals R^{2a′} and R^{2b′} in each case have the above-mentioned meanings. As a divalent metal, magnesium and zinc are preferred; as halogen X, chlorine, bromine and iodine are preferred.

Step q (A-XVI→A-XVII):

Water is added to the double bond in A-XVI in an anti-Markovnikov orientation. For this purpose, the processes that are described under e) are suitable.

Step r (A-XVII→A-XVIII):

The free hydroxy group in A-XVII is protected according to the methods that are known to one skilled in the art. As protective group PG⁶, the protective groups that are known to one skilled in the art, as were already mentioned above for PG⁴ in step a (A-II→A-III), are suitable.

Preferred are those protective groups that can be cleaved under basic or hydrogenolytic reaction conditions, such as, e.g., benzyl, para-nitrobenzyl, acetyl, propionyl, butyryl, and benzoyl radicals. Especially preferred is the benzoyl radical.

Step s (A-XVIII→A-XIX):

Oxidation of the secondary alcohol in A-XVII to ketone A-XIX is carried out according to the conditions that are mentioned under step k). Preferred is oxidation with N-methyl-morpholino-N-oxide with use of tetrapropylammonium perruthenate.

Step t (A-XIX→A-XX):

Protective group PG⁶ in XIX is now selectively cleaved. If this is a hydrogenolytically cleavable protective group, it is preferably hydrogenated in the presence of palladium or platinum catalysts in inert solvents, such as, for example, ethyl acetate or ethanol. If this is a basically cleavable protective group, saponification with carbonates in alcoholic solution, such as, e.g., potassium carbonate in methanol, saponification with aqueous solutions of alkali hydroxides, such as, e.g., lithium hydroxide or sodium hydroxide, are preferably used while employing organic, water-miscible solvents, such as, e.g., methanol, ethanol, tetrahydrofuran or dioxane.

Step u (A-XVII→A-XXI):

Oxidation of alcohols in A-XVII to ketoaldehyde A-XXI is carried out according to the conditions that are mentioned under step k). Preferred is oxidation with N-methyl-morpholino-N-oxide with use of tetrapropylammonium perruthenate as well as the method according to Swem.

Step v (A-XXI→A-XXI):

Oxidation of primary alcohol in A-XX to ketoaldehyde A-XXI is carried out according to the conditions that are mentioned under step k). Preferred is oxidation with N-methyl-morpholino-N-oxide with use of tetrapropylammonium perruthenate.

Step w (A-XXI→A-XXII):

Oxidation of the aldehyde in A-XXI to carboxylic acid A-XXII (R^{13b}=hydrogen)is carried out according to the methods that are known to one skilled in the art. For example, the oxidation according to Jones, oxidation with potassium permanganate, for example in an aqueous system that consists of tert-butanol and sodium dihydrogen phosphate, oxidation with sodium chlorite in aqueous tert-butanol optionally in the presence of a chlorine trap, such as, e.g., 2-methyl-2-butene, can be mentioned.

Oxidation of the aldehyde in A-XXI to ester A-XXII, in which R^{13b} has the above-mentioned meanings and is unequal to hydrogen, can be carried out, for example, with pyridinium dichromate and the desired alcohol HO—R^{13b} in an inert solvent, such as, e.g., dimethylformamide.

Step x (A-VII→A-XXIII):

Oxidation of the primary alcohol in A-VII to aldehyde A-XXIII is carried out according to the conditions that are mentioned under step k). Preferred is oxidation with N-methyl-morpholino-N-oxide with use of tetrapropylammonium perruthenate as well as the method according to Swem.

Step y (A-XXIII→A-XXIV):

Oxidation of aldehyde A-XXIII to carboxylic acid or its esters A-XXIV is carried out according to the conditions already described under w).

Step z (A-XXIV→A-XXV):

Protective group PG⁵ introduced under step d) is cleaved as described under step i.

Step aa (A-XXV→A-XXVI):

Oxidation of the primary alcohol in A-XXV to aldehyde A-XXVI is carried out according to the conditions that are mentioned under step k). Preferred is oxidation with N-methyl-morpholino-N-oxide with use of tetrabutylammonium perruthenate as well as the method according to Swem.

Step ab (A-XXVI→A-XXVII):

The reaction of aldehyde A-XXVI to form alcohols of formula A-XXVII is carried out according to the conditions that are mentioned under step 1).

Step ac (A-XXVII→A-XXII):

Oxidation of the secondary alcohol in A-XXVII to ketone A-XXII is carried out according to the conditions that are mentioned under step k). Preferred is oxidation with N-methyl-morpholino-N-oxide with use of tetrapropylammonium perruthenate.

The compounds of formula A, in which R^{1a′} and R^{1b′} all can have the meanings that are indicated in general formula A, can also be produced from inexpensive or readily available malonic acid dialkyl esters in an efficient way with high optical purity.

The synthesis is described in Diagram 3 below:

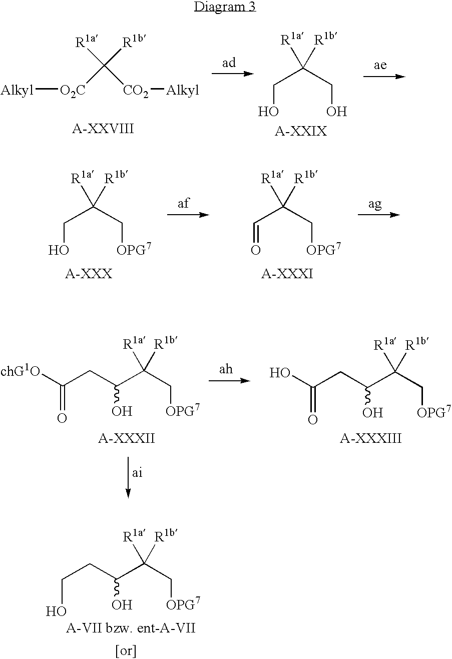

Step ad (A-XXVIII→A-XXIX):

Correspondingly substituted malonic acid ester derivatives A-XXVIII, which are either commercially available or can be produced according to the processes that are known to one skilled in the art from malonic acids or their alkyl esters, are reduced to diols A-XXIX. For this purpose, the reducing agents that are known to one skilled in the art, such as, e.g., diisobutylaluminum hydride, and complex metal hydrides, such as, e.g., lithium aluminum hydride, are suitable.

Step ae (A-XXIX→A-XXX):

A free hydroxyl group in A-XXIX is selectively protected according to the methods that are known to one skilled in the art. As protective group PG⁷, the protective groups that are known to one skilled in the art, as were already mentioned above for PG⁴ in step a (A-II→A-III), are suitable.

Preferred are silicon-containing protective groups.

Step af (A-XXX→A-XXXI):

Oxidation of the remaining, primary hydroxyl group in A-XXX to aldehyde A-XXXI is carried out according to the conditions that are mentioned under step k). Preferred is oxidation with N-methyl-morpholino-N-oxide with use of tetrapropylammonium perruthenate, the use of pyridinium chlorochromate, pyridinium dichromate as well as the method according to Swem.

Step ag (A-XXXI→A-XXXII):

Aldehydes A-XXXI are reacted with an ester of acetic acid chG¹OC(O)CH₃, in which chG¹ means a chiral auxiliary group, in terms of an aldol reaction. Compounds chG¹OC(O)CH₃ are used in optically pure form in the aldol reaction. The type of chiral auxiliary group determines whether the aldol reaction proceeds with high diastereoselectivity or yields a diastereomer mixture that can be separated with physical methods. A survey on comparable diastereoselective aldol reactions is found in Angew. Chem. 99 (1987), 24-37. As chiral auxiliary groups chG¹-OH, for example, optically pure 2-phenyl-cyclohexanol, pulegol, 2-hydroxy-1,2,2-triphenylethanol, and 8-phenylmenthol are suitable.

Step ah (A-XXXII→A-XXXIII):

Diastereomer-pure compounds A-XXXII can then be converted according to the process that is known to one skilled in the art by saponification of the ester unit with simultaneous release of reusable chiral auxiliary component chG¹-OH into enantiomer-pure compounds of type A-XXXIII or ent-A-XXXIII. For saponification, carbonates in alcoholic solution, such as, e.g., potassium carbonate in methanol, aqueous solutions of alkali hydroxides, such as, e.g., lithium hydroxide or sodium hydroxide with use of organic, water-miscible solvents, such as, e.g., methanol, ethanol, tetrahydrofuran or dioxane, are suitable.

Step ai (A-XXXII→A-VIII):

As an alternative to step ah, the chiral auxiliary group can also be removed reductively. In this way, the enantiomer-pure compounds of type A-VIII or ent-A-VIII are obtained. The reduction can be carried out according to the processes that are known to one skilled in the art. As a reducing agent, e.g., diisobutylaluminum hydride and complex metal hydrides, such as, e.g., lithium aluminum hydride, are suitable.

Compounds A-VIII or ent-A-VIII can be converted as previously described into compounds of type A-XIII or ent-A-XIII. Correspondingly, compounds of type A-XXXIII or ent-A-XXXIII can be converted into compounds of type A-XXII or ent-A-XXII according to the processes described above.

As an alternative to the above-described method, the sequence can also be carried out without using chiral auxiliary group chG¹. In this way, racemic mixtures of compounds of type rac-A-VIII or rac-A-XXXIII are then obtained via the corresponding, racemic precursors. These mixtures can in turn be separated according to the processes for racemate cleavage, e.g., chromatography on chiral columns, known to one skilled in the art. The continuation of synthesis can also be carried out with racemic mixtures, however.

Representation of Partial Fragments B:

The synthesis of partial fragments B is described in Diagram 4 below starting from the aldehydes of general formula B-1.

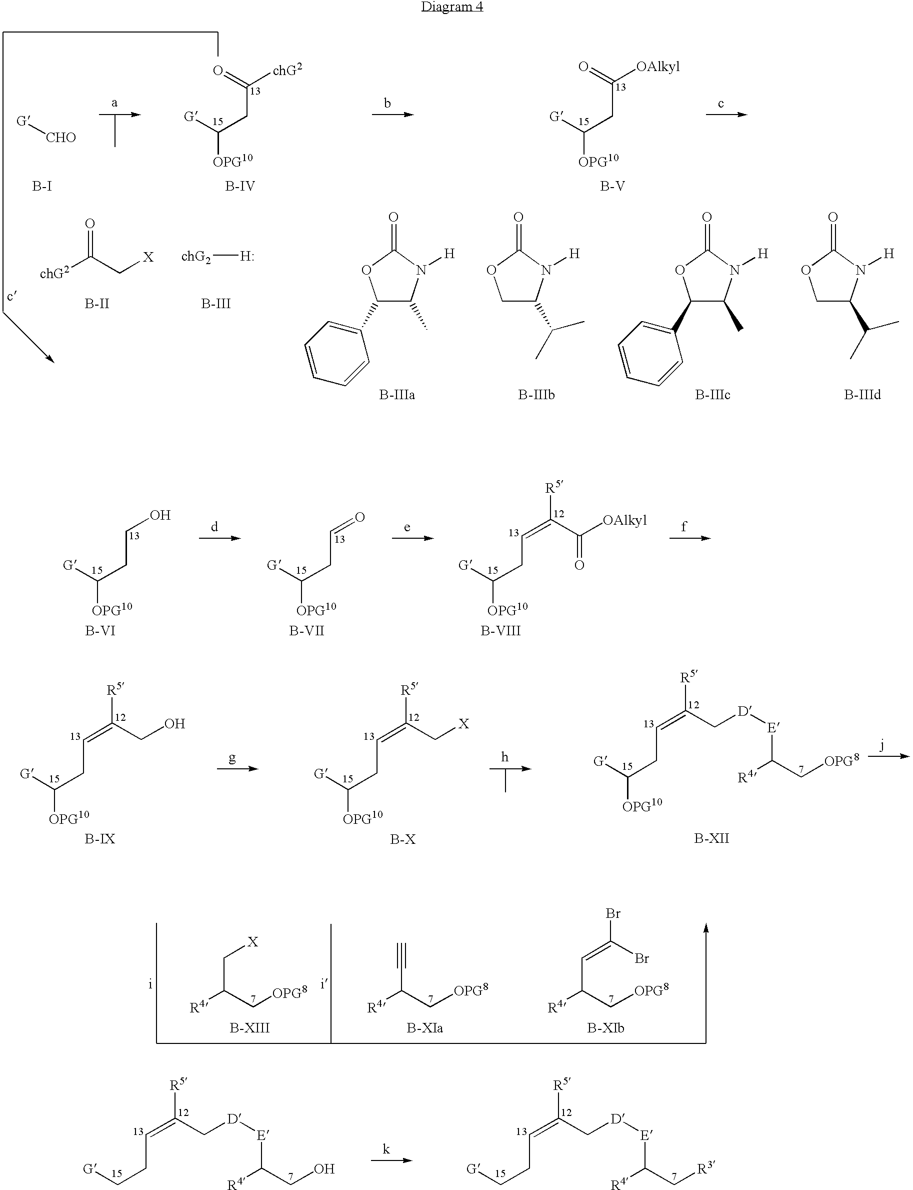

Step a (B-I→B-IV):

Compound B-I is alkylated with the enolate of a carbonyl compound of general formula B-II, in which X is a hydrogen, and chG² is a chiral auxiliary group according to the methods that are known to one skilled in the art. The enolate is produced by action of strong bases, such as, e.g., lithium diisopropylamide or lithium hexamethyldisilazane at low temperatures. Another possibility lies in a kind of Reformatzsky reaction in which the compound of general formula B-II with X=halogen, preferably chlorine or bromine, is converted with CrCl₂ into an organometallic reagent, which then is reacted with aldehyde B-I to form B-IV. As chiral auxiliary group chG²-H (B-III), chiral alcohols that can be produced in an optically pure and inexpensive manner, such as, e.g., pulegol, 2-phenylcyclohexanol, 2-hydroxy-1,2,2-triphenylethanol, 8-phenylmenthol or compounds that contain reactive NH-groups that can be produced in an optically pure and inexpensive manner, such as, e.g., amines, amino acids, lactams or oxazolidinones, are suitable. Preferred are oxazolidinones; especially preferred are the compounds of formulas B-IIIa to B-IIId. The absolute stereochemistry on the α-carbonylcarbon of the compound of general formula B-IV is set by the selection of the respective antipode. In this way, the compounds of general formulas B-IV to B-XV or their respective enantiomers ent-B-IV to ent-B-XV can be obtained in an enantiomer-pure manner. If an achiral alcohol, such as, e.g., ethanol, is used as chG²-H (B-III), the racemic compounds rac-B-IV to rac-B-XV are obtained.

The free hydroxyl group in B-IV is then protected according to the methods that are known to one skilled in the art. As protective groups PG10, the protective groups that are known to one skilled in the art, as were already mentioned above for PG4 in step a (A-II→A-III), are suitable.

Preferred are silicon-containing protective groups, which can be cleaved under acid reaction conditions or with use of fluoride, such as, e.g., trimethylsilyl, triethylsilyl, tert-butyldimethylsilyl, tert-butyldiphenylsilyl, tribenzylsilyl and triisopropylsilyl radicals.

Especially preferred are the tert-butyldiphenylsilyl radical and the tert-butyldimethylsilyl radicals.

Step b (B-IV→B-V):

If group chG² represents one of the chiral auxiliary groups that are mentioned under step a, the latter is recovered by reesterification of B-IV in an alkyl ester of general formula B-V. The reesterification is carried out according to the methods that are known to one skilled in the art. Preferred is reesterification with simple alcohols, such as, e.g., methanol or ethanol in the presence of corresponding titanium(IV) alcoholates.

Step c (B-V→B-VI):

The ester in B-V is reduced to alcohol B-VI. As a reducing agent, the reducing agents that are known to one skilled in the art, such as, e.g., aluminum hydrides, such as, e.g., lithium aluminum hydride or diisobutylaluminum hydride, are suitable. The reaction is carried out in an inert solvent, such as, e.g., diethyl ether, tetrahydrofuran, or toluene.

Step c′ (B-IV→B-VI):

As an alternative to steps b) and c), the carbonyl group in B-IV can be reduced immediately to the alcohols of general formula B-VI according to the conditions that are mentioned under step c). Here, the chiral auxiliary component chG²-H can also be recovered.

Step d (B-VI→B-VII):

The oxidation of the primary alcohol in B-VI to the aldehyde of general formula B-VII is carried out according to the processes that are known to one skilled in the art. For example, oxidation with pyridinium chlorochromate, pyridinium dichromate, the chromium trioxide-pyridine complex, oxidation according to Swem or related methods, e.g., with use of the SO₃— pyridine complex or oxalyl chloride in dimethyl sulfoxide, the use of Dess-Martin periodinane, the use of nitrogen oxides, such as, e.g., N-methyl-morpholino-N-oxide in the presence of suitable catalysts, such as, e.g., tetrapropylammonium perruthenate in inert solvents, can be mentioned. Preferred is the oxidation according to Swem, the SO₃— pyridine complex as well as with N-methyl-morpholino-N-oxide using tetrapropylammonium perruthenate.

Step e (B-VII→B-VIII):

The unsaturated esters of general formula B-VIII are produced by the processes that are known to one skilled in the art. In this respect, methods such as, e.g., the Wittig reaction or the Wittig/Horner reaction, or else the Peterson olefination are suitable. Preferred is the Wittig/Horner reaction with use of phosphonates of type alkylOOC—CHR^{5′}—P(O)(Oalkyl′)₂, whereby alkyl and alkyl′ can be the same or different and preferably mean methyl, ethyl, i-propyl, or trifluoroethyl, and R5′ has the already mentioned meaning, with bases such as, e.g., potassium carbonate, sodium hydride, n-butyllithium, potassium-tert-butanolate, sodium ethanolate, lithium hexamethyldisilazane, sodium hexamethyldisilazane, potassium hexamethyldisilazane and optionally with the additions of, for example, crown ethers, DMPU or HMPA, in solvents such as methanol, tetrahydrofuran, dimethylformamide, diethyl ether; the combination of potassium carbonate in methanol, sodium hydride in dimethylformamide or tetrahydrofuran and potassium hexamethyldisilazane with 18-crown-6 in tetrahydrofuran is preferred.

The E/Z diastereomers that are obtained can be separated, for example, in this stage or in the next step, and can be converted individually per se into the corresponding E- or Z-olefin end products. In the formula diagram, only the E-form is shown for the sake of clarity. All of the following steps also hold true, however, for the corresponding Z-isomer.

Step f (B-VIII→B-IX):

The ester in B-VIII is reduced to alcohol B-IX. As a reducing agent, the reducing agents that are known to one skilled in the art, such as, e.g., aluminum hydrides, such as, e.g., lithium aluminum hydride or diisobutylaluminum hydride, are suitable. The reaction is carried out in an inert solvent, such as, e.g., diethyl ether, tetrahydrofuran, or toluene.

Step g (B-IX→B-X):

The primary hydroxy group in B-IX is converted into a leaving group X in B-X, whereby X can be a grouping that is known to one skilled in the art, such as, for example, mesylate, triflate, nonaflate, chloride, bromide or iodide. In the case of sulfonates, alcohol B-IX is reacted with the corresponding sulfonyl chloride or the corresponding sulfonic acid anhydride in a basic solvent, such as, for example, pyridine, or in a neutral solvent, such as tetrahydrofuran, diethyl ether or methylene chloride with the addition of a base such as pyridine, triethylamine, diisopropylethylamine, sodium hydride or lithium diisopropylamide to form B-X. The halides can be obtained either by using a Finkelstein reaction from the corresponding sulfonates with alkali halides in acetone or else by reaction of alcohol B-IX with iodine, or CCl₄, CBr₄ or else correspondingly substituted ethanes or ethenes in the presence of triphenylphosphine or else bis(diphenylphosphinoethane) and imidazole in an inert solvent, such as, for example, tetrahydrofuran, diethyl ether or methylene chloride.

Step h (B-X→B-XII):

The alkylation of compound B-X is carried out either with the acetylene B-XIa with use of one equivalent of base or else by the dibromoalkene B-XIb with use of at least two equivalents of base in an inert solvent, such as, for example, tetrahydrofuran or diethyl ether, optionally with the addition of DMPU or else HMPA at −80° C. up to 50° C. As suitable bases, e.g., buthyllithium, lithium diisopropylamide or sodium amide can be mentioned.

Another possibility of alkylation would be a copper-catalyzed coupling reaction with the addition of a base, such as, e.g., triethylamine or else potassium carbonate or sodium carbonate in an inert solvent or a mixture of this solvent, such as, for example, diethyl ether, tetrahydrofuran, dimethylformamide or dimethyl sulfoxide. In the case of inorganic bases in the coupling reaction, a commonly used phase-transfer catalyst, such as, for example, tetrabutylammonium bromide, must then be added.

A further reaction is optionally carried out according to the meanings of D′-E′ in this step or in one of the subsequent steps, to the extent that the alkine that is obtained is hydrogenated in a hydrogen atmosphere with the addition of the catalyst that is known to one skilled in the art, such as, for example, palladium, rhodium or else platinum on a vehicle such as carbon, calcium carbonate and barium sulfate or else the Wilkinson catalyst or is reduced by chemical hydrogenation by means of lithium alkanate or diimine, then optionally is converted by the known process of dihydroxylation with osmium tetraoxide with or without chiral catalysts (Sharpless process) into a 1,2-diol or by dioxiram or peracid into the epoxide. Reactive hydroxy groups can optionally be intermediately protected; in this respect, the protective groups already mentioned under step a (A-II→A-III) are considered.

Another alternative for the synthesis of compounds B-XII would be the reaction of compounds of general formula B-X with alkali cyanide or copper cyanide in a polar solvent, such as, for example, dimethylformamide, dimethyl sulfoxide or else DMPU or NMP, followed by a reduction, for example, with diisobutyl aluminum hydride with subsequent hydrolysis to the corresponding aldehyde, and its reaction with the Wittig salt that is generated from compound B-XIII.

Step i (B-IX→B-XH):

If D′-E′ has the meaning of O—CH₂, primary alcohol B-IX is etherified with a sulfonate or halide of general formula B-XIII. In this case, the alcoholate of B-IX is produced by means of a base, such as, for example, sodium hydride, butyllithium or lithium diisopropylamide and reacted in an inert solvent, such as tetrahydrofuran, diethyl ether or dimethylformamide to form B-XII. As an alternative, a phase-transfer-catalyzed etherification in a two-phase system, such as, for example, toluene/sodium hydroxide solution or potassium hydroxide solution with use of a catalyst, such as, e.g., tetrabutylammonium hydrogen sulfate, is also suggested.

Step i′ (B-X→B-XII):

If D′-E′ has the meaning of S—CH₂, SO—CH2 or SO₂—CH₂, the compound of general formula B-X is converted according to the methods that are known to one skilled in the art into a corresponding mercaptan, e.g., by reaction with NaHS or else thioacetate, followed by a saponification. The thus obtained mercaptan is converted into a thioether of formula B-XII analogously to the methods that are described in step i. The latter can optionally be converted into the corresponding sulfoxides or sulfones of formula B-XII by oxidizing agents, such as, for example, H₂O₂/acetonitrile, manganese dioxide, osmium tetraoxide, peracids or sodium periodate in this stage or a later stage.

Step j (B-XII→B-XIV):

If L′=OPG10, protective group PG⁸ is now cleaved according to the process that is known to one skilled in the art. If this is a protective group that can be cleaved acidically, then cleavage can be accomplished with dilute mineral acids in aqueous-alcoholic solutions and with the use of catalytic quantities of acids, such as, e.g., para-toluenesulfonic acid, para-toluenesulfonic acid-pyridinium salt, or camphorsulfonic acid in alcoholic solutions, preferably in ethanol or isopropanol.

If L is to be an NR₂₂ group in the end compounds of formula I, first protective group PG 10 is cleaved selectively before the cleavage of the protective group PG 8 according to the methods that are known to one skilled in the art (also see above in this respect). The thus obtained secondary alcohol is converted with a sulfonyl chloride or a sulfonic acid anhydride into a sulfonate and optionally then in a Finkelstein reaction with an alkali bromide or alkali chloride, or by reaction of the secondary alcohol with CBr₄ in the presence of triphenylphosphine or bis(diphenylphosphinoethane) into a secondary halide. The thus obtained halides or.sulfonates can then be converted into a corresponding azide (L′=N₃) by a nucleophilic substitution with, e.g., sodium azide in a neutral polar solvent, such as, for example, dimethylformamide or dimethyl sulfoxide. The above-described cleavage of protective group PG⁸ would then follow.

Step k (B-XIV→B-XV):

The oxidation of the primary alcohol in B-XIV to the corresponding aldehyde is carried out according to the processes that are known to one skilled in the art. For example, oxidation with pyridinium chlorochromate, pyridinium dichromate, chromium trioxide-pyridine complex, oxidation according to Swem or related methods, e.g., with use of the SO₃-pyridine complex or oxalyl chloride in dimethyl sulfoxide, the use of Dess-Martin periodinane, the use of nitrogen oxides, such as, e.g., N-methyl-morpholino-N-oxide in the presence of suitable catalysts, such as, e.g., tetrapropylammonium perruthenate in inert solvents, can be mentioned. Preferred is the oxidation according to Swem, as well as with N-methyl-morpholino-N-oxide using tetrapropylammonium perruthenate.

If R3′≠H, the corresponding secondary alcohol can now be produced according to the methods that are known to one skilled in the art with organometallic compounds of general formula M—R³, in which M stands for an alkali metal, preferably lithium, or a divalent metal MX, in which X represents a halogen, and radical R^{3′}has the above-mentioned meaning. As a divalent metal, magnesium and zinc are preferred; as halogen X, chlorine, bromine and iodine are preferred.

The thus obtained secondary alcohol is converted by oxidation into the ketone of general formula B-XV with R3′≠H according to the process initially mentioned under k). The oxidation with N-methyl-morpholino-N-oxide with use of tetrapropylammonium perruthenate is preferred.

Representation of Compounds B-XI and B-XIII:

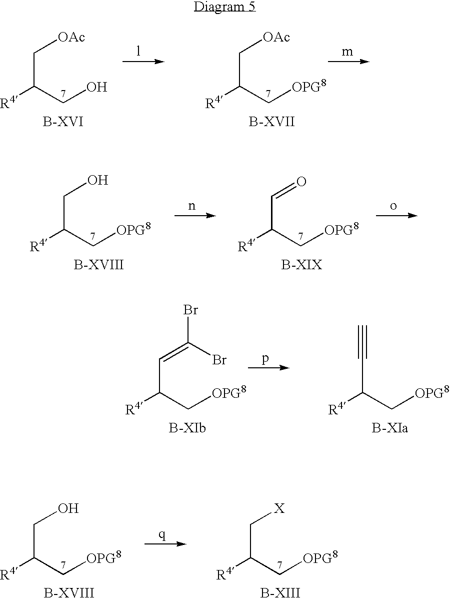

Step 1 (B-XVI→B-XVII):

The racemic starting materials are known or can be produced easily from the correspondingly substituted malonic esters by reduction and partial acetate formation. The production of the chiral starting substances of general formula B-XVI are either known or can be produced as described in, e.g., Tetrahedron Letters 27, 5707, starting from the corresponding prochiral diols by enzymatic acylation or starting from the prochiral diacetates by enzymatic hydrolysis.

The free hydroxyl group in B-XVI is protected according to the methods that are known to one skilled in the art. As protective group PG⁸, the protective groups that are known to one skilled in the art, as they were already mentioned above for PG⁴ in step a (A-II→A-III), are suitable.

Preferred are those protective groups that can be cleaved under acidic reaction conditions, such as, e.g., the methoxymethyl, tetrahydropyranyl, tetrahydrofuranyl, or trimethylsilyl radical.

Especially preferred is the tetrahydropyranyl radical.

Step m (B-XVII→B-XVIII):

The saponification of the acetate can be carried out by treatment with dilute sodium hydroxide solution or potassium hydroxide solution or by reesterification with potassium carbonate in methanol. An alternative would also be a reduction with modified aluminum hydrides, such as, for example, diisobutylaluminum hydride in an inert solvent, such as, e.g., toluene at −80° to 0° C.

Step n (B-XVIII→B-XIX):

The oxidation of the primary alcohol in B-XVIII to aldehyde B-XIX is carried out according to the processes that are known to one skilled in the art. For example, oxidation with pyridinium chlorochromate, pyridinium dichromate, the chromium trioxide-pyridine complex, oxidation according to Swern or related methods, e.g., with use of the SO₃-pyridine complex or oxalyl chloride in dimethyl sulfoxide, the use of Dess-Martin periodinane, the use of nitrogen oxides, such as, e.g., N-methyl-morpholino-N-oxide in the presence of suitable catalysts, such as, e.g., tetrapropylammonium perruthenate in inert solvents, can be mentioned. Preferred is the oxidation according to Swem, as well as with N-methyl-morpholino-N-oxide using tetrapropylammonium perruthenate.

Step o (B-XIX→B-XIb):

Aldehyde B-XIX is reacted to form alkene B-XIB according to the processes, known to one skilled in the art, with dibromomethylene phosphorane, generated from a mixture that consists of tetrabromomethane in the presence of zinc powder and triphenylphosphine.

Step p (B-XIb→B-XIa):

The dibromoalkene B-XIB can be converted into the alkine B-XIa by treatment with two equivalents of base, such as, for example, butyllithium, lithium diisopropylamide or sodium amide in an inert solvent such as tetrahydrofuran or diethyl ether.

Step q (B-XVIII→B-XIII):

The production of compounds of general formula B-XIII is carried out by the conversion of the primary hydroxy group in B-XVIII into a leaving group X in B-XIII, whereby X can be a grouping that is known to one skilled in the art, such as, for example, mesylate, triflate, nonaflate, chloride, bromide or iodide. In the case of sulfonates, the alcohol B-XVIII is reacted to form B-XIII with the corresponding sulfonyl chloride or the corresponding sulfonic acid anhydride in a basic solvent, such as, for example, pyridine, or in a neutral solvent, such as tetrahydrofuran, diethyl ether or methylene chloride with the addition of a base such as pyridine, triethylamine, diisopropylethylamine, sodium hydride or lithium diisopropylamide. The halides can be obtained either by using a Finkelstein reaction from the corresponding sulfonates with alkali halides in acetone or else by reaction with iodine or CCl₄, CBr4 or else correspondingly substituted ethane or ethenes in the presence of triphenylphosphine or else bis(diphenylphosphinoethane) and imidazole in an inert solvent, such as, for example, tetrahydrofuran, diethyl ether or methylene chloride.

If R^{4′}=Me, the synthesis of the corresponding component B-XVIII can also be carried out starting from the commercially available (S)- or (R)-3-hydroxy-2-methylpropionic acid methyl ester.

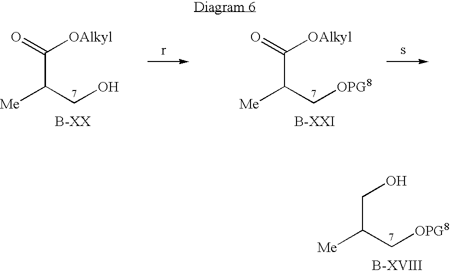

Step r (B-XX→B-XXI):

The free hydroxyl group in B-XX is protected according to the methods that are known to one skilled in the art. As protective group PG⁸, the protective groups that are known to one skilled in the art, as were already mentioned above for PG⁴ in step a (A-II→A-III), are suitable.

Preferred are those protective groups that can be cleaved under acidic reaction conditions, such as, e.g., the methoxymethyl, tetrahydropyranyl, tetrahydrofuranyl, and trimethylsilyl radical.

Especially preferred is the tetrahydropyranyl radical.

Step s (B-XXI→B-XXII):

The ester in B-XXI is reduced to alcohol B-XXII. As a reducing agent, the reducing agents that are known to one skilled in the art, such as, e.g., aluminum hydrides, such as, e.g., lithium aluminum hydride or diisobutylaluminum hydride, are suitable. The reaction is carried out in an inert solvent, such as, e.g., diethyl ether, tetrahydrofuran, or toluene.

If G represents the group

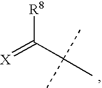

the corresponding alcohols of formula B-VI in partial fragment B can also be produced as follows.

The substances of formula B-VI, whereby G represents the group

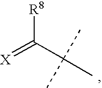

can be produced in an efficient way with high optical purity (>99.5%) from inexpensive malic acid that can be obtained at a reasonable price.

The synthesis is described in Diagrarn 7 below in the example of L-(−)-malic acid (B-XXII). Starting from D(+)-malic acid (ent-B-XXII), the corresponding enantiomeric compounds (ent-B-XXIII to ent-B-V) are obtained, and starting from racemic malic acid (rac-B-XII), the corresponding racemic compounds (rac-B-XXIII to rac-B-VI) are obtained.

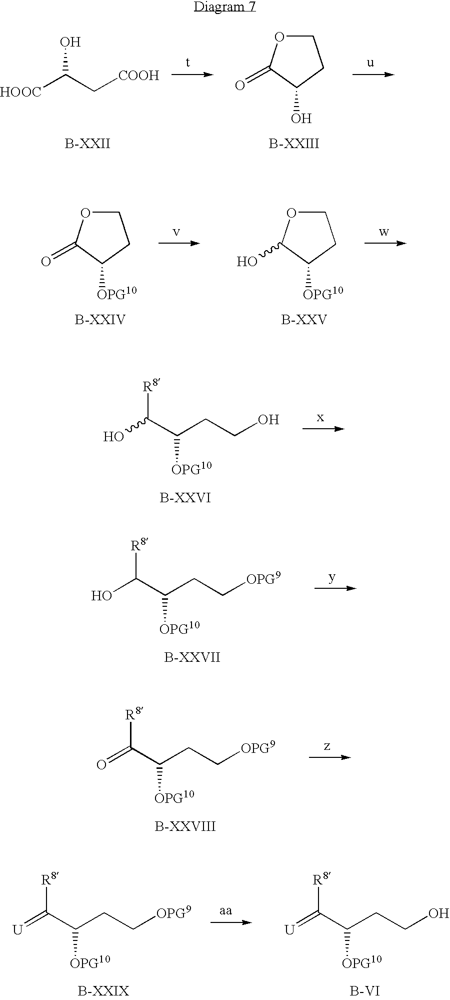

Step t (Malic acid B-XXII→B-XXIII):

L-(−)-Malic acid is converted into hydroxylactone B-XXIII according to a process that is known in the literature (Liebigs Ann. Chem. 1993, 1273-1278).

Step u (B-XXIII→B-XXIV):

The free hydroxy group in compound B-XXIII is protected according to the methods that are known to one skilled in the art. As protective group PG10, the protective groups that are known to one skilled in the art, as were already mentioned above for PG4 in step a (A-II→AIII), are suitable.

Preferred are those protective groups that can be cleaved under the action of fluoride, but are stable under weakly acidic reaction conditions, such as, e.g., the tert-butyldiphenylsilyl, tert-butyldimethylsilyl or triisopropylsilyl radical.

Especially preferred are the tert-butyldiphenylsilyl radical and the tert-butyldimethylsilyl radical.

Step v (B-XXIV→B-XXV):

Lactone B-XXIV is reduced to lactol B-XXV according to the methods that are known to one skilled in the art. As reducing agents, aluminum hydrides that are modified in their reactivity, such as, e.g., diisobutylaluminum hydride, are suitable. The reaction is carried out in an inert solvent, such as, e.g., toluene, preferably at low temperatures (−20 to −100° C.).

Step w (B-XXV→B-XXVI):

The reaction of lactol B-XXV to compounds of formula B-XXVI is carried out with organometallic compounds of general formula M-R^{8′}, in which M stands for an alkali metal, preferably lithium, or a divalent metal MX, in which X represents a halogen, and R^{8′} has the above-mentioned meanings. As a divalent metal, magnesium and zinc are preferred, and as halogen X, chlorine, bromine and iodine are preferred.

Step x (B-XXVI→B-XXVII):

The primary hydroxyl group in compound B-XXVI is protected in a selective manner relative to the secondary hydroxyl group according to the methods that are known to one skilled in the art.

The secondary hydroxy group is optionally then protected also according to the methods that are familiar to one skilled in the art.

As protective groups PG⁹ the protective groups that are known to one skilled in the art, as were already mentioned above for PG⁴ in step a (A-II→A-III), are suitable.

Preferred are those protective groups that can be cleaved under weakly acidic reaction conditions in a selective manner in the presence of protective group PG10, such as, e.g., the trimethylsilyl, triethylsilyl, or tert-butyldimethylsilyl radical.

Especially preferred is the tert-butyldimethylsilyl radical.

Step y (B-XXVII→B-XXVIII):

Oxidation of the secondary alcohol in B-XXVII to ketone B-XXVIII is carried out according to the methods that are known to one skilled in the art. For example, oxidation with pyridinium chlorochromate, pyridinium dichromate, or the chromium trioxide-pyridine complex, oxidation according to Swem or related methods, e.g., with use of oxalyl chloride in dimethyl sulfoxide, the use of Dess-Martin periodinane, the use of nitrogen oxides, such as, e.g., N-methyl-morpholino-N-oxide in the presence of suitable catalysts, such as, e.g., tetrapropylammonium perruthenate in inert solvents, can be mentioned. Preferred is oxidation according to Swem.

Step z (B-XXVIII→B-XXIX):

For compounds in which U is equal to CR10′R11′, this grouping is established according to the processes that are known to one skilled in the art. For this purpose, methods such as, e.g., the Wittig or Wittig/Homer reaction, or the addition of an organometallic compound MCHR10′R11′ with dehydration, are suitable. Preferred are the Wittig and Wittig/Homer reactions with use of phosphonium halides of type CR10′R11′P(Ph)3⁺ Hal⁻ or phosphonates of type CR10′R11′P(O)(Oalkyl)2 with Ph equal to phenyl, R10′, R11′ and halogen in the already mentioned conditions with strong bases, such as, e.g., n-butyllithium, potassium-tert-butanolate, sodium ethanolate, sodium hexamethyldisilazane; n-butyllithium is preferred as a base.

For compounds in which U represents two alkoxy groups OR¹⁹ or a C₂-C₁₀-alkylene-α,ω-dioxy group, the ketone is ketalized under acid catalysis according to the methods that are known to one skilled in the art, for example, with use of an alcohol HOR¹⁹ or a C₂-C₁₀-alkylene-α,ω-diol.

Step aa (B-XXIX→B-VI):

Protective group PG⁹ that is introduced under x is now cleaved in a selective manner in the presence of PG¹⁰ according to the processes that are known to one skilled in the art. If this is a protective group that can be cleaved acidically, then cleavage can be accomplished preferably under weakly acidic conditions, such as, e.g., by reaction with dilute organic acids in inert solvents. Acetic acid is preferred.

Representation of Partial Fragments AB and Their Cyclization to I:

Partial fragments of general formula AB AB,

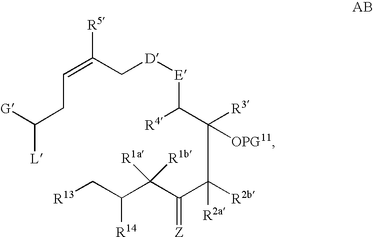

in which R^{1a′}, R^{1b′}, R^{2a′}, R^{2b′}, R^{3′}, R^{4′}, R^{5′}, R¹³, R¹⁴, D′, E′, L′, G′ and Z have the meanings that are already mentioned, are obtained from previously described fragments A and B according to the process that is shown in Diagram 8.

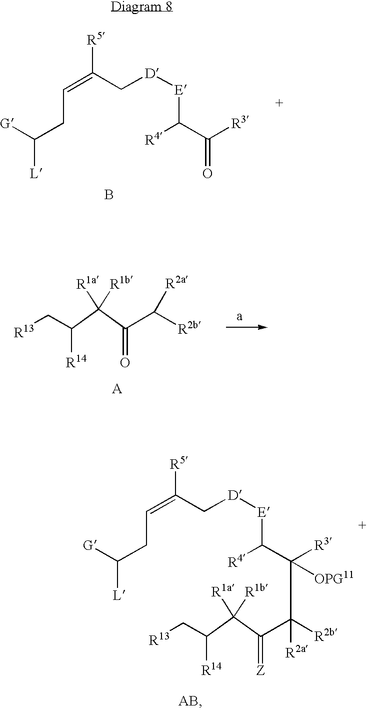

Step a (A+B→AB):

Compound B is alkylated with the enolate of a carbonyl compound of general formula A. The enolate is produced by action of strong bases, such as, e.g., lithium diisopropylamide and lithium hexamethyldisilazane, at low temperatures.

From thus obtained fragments AB, the different compounds of general formula I can then be produced as follows:

Step b (AB→I):

Compounds AB, in which R¹³ represents a carboxylic acid CO₂H and L′ represents a hydroxy group, are reacted according to the methods, known to one skilled in the art, for the formation of large macrolides to form compounds of formula I, in which Y has the meaning of an oxygen atom. The method that is described in “Reagents for Organic Synthesis, Vol. 16, p. 353” with use of 2,4,6-trichlorobenzoic acid chloride and suitable bases, such as, e.g., triethylamine, 4-dimethylaminopyridine, and sodium hydride, is preferred.

Step c (AB→I):

Compounds AB, in which R¹³ represents a group CH₂OH and L′ represents a hydroxy group, can preferably be reacted with use of triphenylphosphine and azodiesters, such as, for example, azodicarboxylic acid diethyl esters, to form compounds of formula I, in which Y has the meaning of two hydrogen atoms.

Compounds AB, in which R¹³ represents a group CH₂OSO₂alkyl or CH₂OSO₂aryl or CH₂OSO₂aralkyl and L′ represents a hydroxy group, can be cyclized after deprotonation with suitable bases, such as, for example, sodium hydride, n-butyllithium, 4-dimethylaminopyridine, Hünig base, and alkali hexamethyldisilazanes, to form compounds of general formula I, in which Y has the meaning of two hydrogen atoms.

Step d (AB→I):

Compounds AB, in which R¹³ represents a carboxylic acid CO₂H and L′ represents an azide, are reacted first according to the methods that are known to one skilled in the art for the formation of amines from azides, for example with triphenylphosphine in the presence of or by later addition of water, or by other reductive methods, such as, for example, tin(I) chloride in methanol. If R22 is not equal to hydrogen, the corresponding alkyl radical optionally can be introduced by a reductive amination. The cyclization to the large lactam ring of formula I, in which L has the meaning of NR²² and Y has the meaning of an oxygen atom, can be carried out, for example, by reaction with diphenyl phosphorylazide with the addition of bases to an inert solvent, such as, e.g., the combination of sodium bicarbonate in dimethylformamide.

Step e (AB→I):

Compounds AB, in which R¹³ represents a group CH₂OH and L′ represents an azide, are reacted first according to the methods that are known to one skilled in the art for the formation of amines from azides, for example with triphenylphosphine in the presence of water, or by other reductive methods, such as, for example, tin(II) chloride in methanol. If R²² is not equal to hydrogen, the corresponding alkyl radical optionally can be introduced by a reductive amination. The cyclization can be carried out after oxidation of the primary hydroxyl group to the corresponding aldehyde, followed by another reductive amination, by which then compounds of formula I are obtained, in which L has the meaning of NR²², and Y has the meaning of two hydrogen atoms.

Free hydroxyl groups in I, A, B, and AB can be further functionally modified by etherification or esterification, free carbonyl groups by ketalization, enol ether formation or reduction, triple and double bonds by hydrogenations or oxidations.

The invention relates to all stereoisomers of these compounds and also mixtures thereof.

In addition, the invention relates to all prodrug formulations of these compounds, i.e., all compounds that release in vivo a bioactive active ingredient component of general formula I.

Biological Actions and Applications of the New Derivatives:

The new compounds of formula I are valuable pharmaceutical agents. They interact with tubulin by stabilizing microtubuli that are formed and are thus able to influence the cell-splitting in a phase-specific manner. This relates mainly to quick-growing, neoplastic cells, whose growth is largely unaffected by intercellular regulating mechanisms. Active ingredients of this type are in principle suitable for treating malignant tumors. As applications, there can be mentioned, for example, the treatment of ovarian, stomach, colon, adeno-, breast, lung, head and neck carcinomas, malignant melanoma, acute lymphocytic and myelocytic leukemia. The compounds according to the invention are suitable owing to their properties basically for anti-angiogenesis therapy as well as for treatment of chronic inflammatory diseases, such as, for example, psoriasis or arthritis. To avoid uncontrolled proliferation of cells and for better compatibility of medical implants, they can basically be applied or introduced into the polymer materials that are used for this purpose. The compounds according to the invention can be used alone or to achieve additive or synergistic actions in combination with other principles and classes of substances that can be used in tumor therapy.

As examples, there can be mentioned the combination with

Platinum complexes, such as, e.g., cis-platinum, carboplatinum,

intercalating substances, e.g., from the class of anthracyclines, such as, e.g., doxorubicin or from the class of anthrapyrazoles, such as, e.g., C1-941,

substances that interact with tubulin, e.g., from the class of vinca-alkaloids, such as, e.g., vincristine, vinblastine or from the class of taxanes, such as, e.g., taxol, taxotere or from the class of macrolides, such as, e.g., rhizoxin or other compounds, such as, e.g., colchicine, combretastatin A-4, discodermolide and its analogs,

DNA topoisomerase inhibitors, such as, e.g., camptothecin, etoposide, topotecan, teniposide,

folate- or pyrimidine-antimetabolites, such as, e.g., lometrexol, gemcitubin,

DNA-alkylating compounds, such as, e.g., adozelesin, dystamycin A,

inhibitors of growth factors (e.g., of PDGF, EGF, TGFb, EGF), such as, e.g., somatostatin, suramin, bombesin antagonists,

inhibitors of protein tyrosine kinases or protein kinases A or C, such as, e.g., erbstatin, genistein, staurosporine, ilmofosine, 8-Cl-cAMP,

antihormones from the class of antigestagens, such as, e.g., mifepristone, onapristone or from the class of antiestrogens, such as, e.g., tamoxifen or from the class of antiandrogens, such as, e.g., cyproterone acetate,

metastases-inhibiting compounds, e.g., from the class of eicosanoids, such as, e.g., PGl₂, PGE₁, 6-oxo-PGE₁ as well as their more stable derivatives (e.g., iloprost, cicaprost, misoprostol),

inhibitory, oncogenic RAS proteins, which influence the mitotic signal transduction, such as, for example, inhibitors of the farnesyl-protein-transferase,

natural or synthetically produced antibodies, which are directed against factors or their receptors, which promote tumor growth, such as, for example, the erbB2 antibodies.

The invention also relates to pharmaceutical agents that are based on pharmaceutically compatible compounds, i.e., compounds of general formula I that are nontoxic in the doses used, optionally together with commonly used adjuvants and vehicles.

The compounds according to the invention can be encapsulated with liposomes or enclosed in an α-, β-, or γ-cyclodextrin clathrate.

According to methods of galenicals that are known in the art, the compounds according to the invention can be processed into pharmaceutical preparations for enteral, percutaneous, parenteral or local administration. They can be administered in the form of tablets, coated tablets, gel capsules, granulates, suppositories, implants, injectable, sterile, aqueous or oily solutions, suspensions or emulsions, ointments, creams and gels.

In this case, the active ingredient or ingredients can be mixed with the adjuvants that are commonly used in galenicals, such as, e.g., gum arabic, talc, starch, mannitol, methyl cellulose, lactose, surfactants such as Tweens or Myrj, magnesium stearate, aqueous or non-aqueous vehicles, paraffin derivatives, cleaning agents, dispersing agents, emulsifiers, preservatives and flavoring substances for taste correction (e.g., ethereal oils).

The invention thus also relates to pharmaceutical compositions that as active ingredients contain at least one compound according to the invention. A dosage unit contains about 0.1-100 mg of active ingredient(s). In humans, the dosage of the compounds according to the invention is approximately 0.1-1000 mg per day.

The examples below are used for a more detailed explanation of the invention, without intending that it be limited to these examples:

### EXAMPLE 1

(4S,7R,8S,9S,13E,16S(E))-4,8-Dihydroxy-13-fluoro-16-(1-methyl-2-(2-methyl-4-thiazolyl)ethenyl)-1,11-dioxa-5,5,7,9-tetramethyl-cyclohexadec-13-ene-2,6-dione

### EXAMPLE 1a

(3S)-1-Oxa-2-oxo-3-(tetrahydropyran-2(RS)-yloxy)-4,4-dimethyl-cyclopentane

The solution of 74.1 g (569 mmol) of D-(−)-pantolactone in 11 of anhydrous dichloromethane is mixed with 102 ml of 3,4-dihydro-2H-pyran, and 2 g of p-toluenesulfonic acid-pyridinium salt under an atmosphere of dry argon, and it is stirred for 16 hours at 23° C. It is poured into a saturated sodium bicarbonate solution, the organic phase is separated and dried on sodium sulfate. After filtration and removal of the solvent, the residue is chromatographed on about 5 kg of fine silica gel with a mixture that consists of n-hexane and ethyl acetate. 119.6 g (558 mmol, 98%) of the title compound is isolated as a colorless oil.

¹H-NMR (CDCl₃): δ=1.13 (3H), 1.22 (3H), 1.46-1.91 (6H), 3.50-3.61 (1H), 3.86 (1H), 3.92 (1H), 4.01 (1H), 4.16 (1H), 5.16 (1H) ppm.

### EXAMPLE 1b

(2RS,3S)-1-Oxa-2-hydroxy-3-(tetrahydropyran-2(RS)-yloxy)-4,4-dimethyl-cyclopentane

The solution of 117.5 g (548 mmol) of the compound, presented according to Example 1 a, in 2.4 l of anhydrous toluene is cooled under an atmosphere of dry argon to −70° C., mixed within 1 hour with 540 ml of a 1.2 molar solution of diisobutylaluminum hydride in toluene, and it is stirred for 3 more hours at −70° C. It is allowed to heat to −20° C., mixed with saturated ammonium chloride solution, water, and the precipitated aluminum salts are separated by filtration on Celite. The filtrate is washed with water and saturated sodium chloride solution and dried on magnesium sulfate. After filtration and removal of the solvent, 111.4 g (515 mmol, 94%) of the title compound is isolated as a colorless oil, which is further reacted without purification.

IR(CHCl₃): 3480, 3013, 2950, 2874, 1262, 1133, 1074, 1026 and 808 cm⁻¹.

### EXAMPLE 1c

(3S)-2,2-Dimethyl-3-(tetrahydropyran-2(R)-yloxy)-pent4-en-1-ol and (3S)-2,2-dimethyl-3-(tetrahydropyran-2(S)-yloxy)-pent4-en-1-ol

The suspension of 295 g of methyl-triphenylphosphonium bromide in 2.5 l of anhydrous tetrahydrofuran is mixed under an atmosphere of dry argon at −60° C. with 313 ml of a 2.4 molar solution of n-butyllithium in n-hexane, allowed to heat to 23° C., stirred for one more hour and cooled to 0° C. It is mixed with the solution of 66.2 g (306 mmol) of the compound, presented according to Example 2, in 250 ml of tetrahydrofuran, allowed to heat to 23° C. and stirred for 18 hours. It is poured into a saturated sodium bicarbonate solution, extracted several times with dichloromethane, and the combined organic extracts are dried on sodium sulfate. After filtration and removal of the solvent, the residue is chromatographed on about 5 l of fine silica gel with a gradient system that consists of n-hexane and ethyl acetate. 36.5 g (170 mmol, 56%) of the nonpolar THP-isomer of the title compound, 14.4 g (67.3 mmol, 22%) of the polar THP-isomer of the title compound, as well as 7.2 g (33.3 mmol; 11%) of the starting material are isolated in each case as a colorless oil.

¹H-NMR (CDCl₃), nonpolar isomer: δ=0.78 (3H), 0.92 (3H), 1.41-1.58 (4H), 1.63-1.87 (2H), 3.18 (1H), 3.41 (1H), 3.48 (1H), 3.68 (1H), 3.94 (1H), 4.00 (1H), 4.43 (1H), 5.19 (1H), 5.27 (1H), 5.75 (1H) ppm.

¹H-NMR (CDCl₃), polar isomer: δ=0.83 (3H), 0.93 (3H), 1.42-1.87 (6H), 2.76 (1H), 3.30 (1H), 3.45 (1H), 3.58 (1H), 3.83 (1H), 3.89 (1H), 4.65 (1H), 5.12-5.27 (2H), 5.92 (1H) ppm.

### EXAMPLE 1d

(3S)-1-(tert-Butyldiphenylsilyloxy)-2,2-dimethyl-pentane-3-(tetrahydropyran-2-yloxy)-pent-4-ene

The solution of 59.3 g (277 mmol) of the THP-isomer mixture, presented according to Example 1 c, in 1000 ml of anhydrous dimethylformamide is mixed under an atmosphere of dry argon with 28 g of imidazole and 85 ml of tert-butyldiphenylchlorosilane, and it is stirred for 16 hours at 23° C. It is poured into water, extracted several times with dichloromethane, the combined organic extracts are washed with water and dried on sodium sulfate. After filtration and removal of the solvent, the residue is chromatographed on fine silica gel with a gradient system that consists of n-hexane and ethyl acetate. 106.7 g (236 mmol, 85%) of the title compound is isolated as a colorless oil.

¹H-NMR (CDCl₃): δ=0.89 (3H), 0.99 (3H), 1.08 (9H), 1.34-1.82 (6H), 3.40 (1H), 3.51 (2H), 3.76 (1H), 4.02 (1H), 4.67 (1H), 5.18 (1H), 5.23 (1H), 5.68 (1H), 7.30-7.48 (6H), 7.60-7.73 (4H) ppm.

### EXAMPLE 1e

(3S)-1-(tert-Butyldiphenylsilyloxy)-2,2-dimethyl-3-(tetrahydropyran-2-yloxy)-pentan-5-ol

The solution of 3.09 g (6.83 mmol) of the compound, presented according to Example 1d, in 82 ml of tetrahydrofuran is mixed with 13.1 ml of a 1 molar solution of borane in tetrahydrofuran under an atmosphere of dry argon at 23° C., and it is allowed to react for 1 hour. Then, while being cooled with ice, it is mixed with 16.4 ml of a 5% sodium hydroxide solution as well as 8.2 ml of a 30% hydrogen peroxide solution, and it is stirred for another 30 minutes. It is poured into water, extracted several times with ethyl acetate, the combined organic extracts are washed with water, saturated sodium chloride solution and dried on magnesium sulfate. The residue that is obtained after filtration and removal of the solvent is purified by chromatography on fine silica gel with a gradient system that consists of n-hexane and ethyl acetate. 1.78 g (3.78 mmol, 55%) of the title compound is isolated as a chromatographically separable mixture of the two THP-epimers, as well as 0.44 g (1.14 mmol, 17%) of the title compound of Example 6 in each case as a colorless oil.

¹H-NMR (CDCl₃), nonpolar THP-isomer: δ=0.80 (3H), 0.88 (3H), 1.10 (9H), 1.18-1.80 (9H), 3.27 (1H), 3.39 (1H), 3.48 (1H), 3.64 (1H), 3.83 (1H), 3.90-4.08 (2H), 4.49 (1H), 7.31-7.50 (6H), 7.58-7.73 (4H) ppm.

¹H-NMR (CDCl₃), polar THP-isomer: δ=0.89 (3H), 0.98 (3H), 1.08 (9H), 1.36-1.60 (4H), 1.62-1.79 (3H), 1.88 (1H), 2.03 (1H), 3.37 (1H), 3.50 (1H), 3.57 (1H), 3.62-3.83 (4H), 4.70 (1H), 7.30-7.48 (6H), 7.61-7.73 (4H) ppm.

### EXAMPLE 1f

(3S)-1-(tert-Butyldiphenylsilyloxy)-2,2-dimethyl-3-hydroxy-pent4-ene

The solution of 106.7 g (236 mmol) of the compound, presented according to Example 1d, in 1.5 l of anhydrous ethanol is mixed with 5.9 g of pyridinium-p-toluenesulfonate under an atmosphere of dry argon, and it is heated for 6 hours to 50° C. After removal of the solvent, the residue is chromatographed on fine silica gel with a mixture that consists of n-hexane and ethyl acetate. 82.6 g (224 mmol, 95%) of the title compound is isolated as a colorless oil, in which in addition about 5 g of ethoxy-tetrahydropyran is contained.

¹H-NMR (CDCl₃) of an analytic sample: δ=0.89 (6H), 1.08 (9H), 3.45 (1H), 3.49 (1H), 3.58 (1H), 4.09 (1H), 5.21 (1H), 5.33 (1H), 5.93 (1H), 7.34-7.51 (6H), 7.63-7.73 (4H) ppm.

### EXAMPLE 1g

(3S)-1-(tert-Butyldiphenylsilyloxy)-2,2-dimethyl-pentane-3,5-diol

Analogously to Example 1e, the solution of 570 mg (1.55 mmol) of the compound that is presented according to Example 1f is reacted, and after working-up and purification, 410 mg (1.06 mmol, 68%) of the title compound is isolated as a colorless oil.

¹H-NMR (CDCl₃): δ=0.82 (3H), 0.93 (3H), 1.08 (9H), 1.56-1.79 (2H), 3.11 (1H), 3.50 (2H), 3.78-3.92 (3H), 4.02 (1H), 7.34-7.51 (6H), 7.61-7.71 (4H) ppm.

### EXAMPLE 1h

4(S)-[2-Methyl-1-(tert-butyldiphenylsilyloxy)-prop-2-yl]-2,2-dimethyl-[1,3] dioxane

The solution of 100 mg (0.212 mmol) of the compounds, presented according to Example 1e, in 2.6 ml of anhydrous acetone is mixed with 78.9 mg of copper(I) sulfate, a spatula tip full of p-toluenesulfonic acid-monohydrate under an atmosphere of dry argon, and it is stirred for 16 hours at 23° C. It is mixed with saturated sodium bicarbonate solution, extracted several times with diethyl ether, washed with saturated sodium chloride solution and dried on sodium sulfate. The residue that is obtained after filtration and removal of the solvent is purified by chromatography on fine silica gel with a gradient system that consists of n-hexane and ethyl acetate. 24 mg (56 μmol, 27%) of the title compound is isolated as a colorless oil.

¹H-NMR (CDCl₃): δ=0.83 (3H), 0.89 (3H), 1.07 (9H), 1.30 (1H), 1.36 (3H), 1.44 (3H), 1.71 (1H), 3.24 (1H), 3.62 (1H), 3.86 (1H), 3.91-4.03 (2H), 7.31-7.48 (6H), 7.61-7.74 (4H) ppm.

Variant II

320 mg (0.88 mmol) of the compound that is presented according to Example 6 is reacted analogously to Example 1h, variant 1, and after working-up and purification, 234 mg (0.548 mmol, 62%) of the title compound is isolated.

Variant III

The solution of 5.60 g (14.5 mmol) of the compound, presented according to Example 1g, in 250 ml of anhydrous dichloromethane, is mixed with 10 ml of 2,2-dimethoxypropane, 145 mg of camphor-10-sulfonic acid under an atmosphere of dry argon, and it is stirred for 6 hours at 23° C. It is mixed with triethylamine, diluted with ethyl acetate, washed with saturated sodium bicarbonate solution and dried on sodium sulfate. After filtration and removal of the solvent, the residue is chromatographed on fine silica gel with a mixture that consists of n-hexane and ethyl acetate. 5.52 g (12.9 mmol, 89%) of the title compound is isolated as a colorless oil.

### EXAMPLE 1i

(4S)4-(2-Methyl-1-hydroxy-prop-2-yl)-2,2-dimethyl-[1,3]dioxane

The solution of 5.6 g (13.1 mmol) of the compound, presented according to Example 1h, in 75 ml of anhydrous tetrahydrofuran is mixed under an atmosphere of dry argon with 39 ml of a 1 molar solution of tetrabutylammonium fluoride in tetrahydrofuran, and it is heated for 16 hours to 50° C. It is mixed with saturated sodium bicarbonate solution, extracted several times with ethyl acetate, washed with saturated sodium chloride solution and dried on sodium sulfate. The residue that is obtained after filtration and removal of the solvent is purified by chromatography on fine silica gel with a gradient system that consists of n-hexane and ethyl acetate. 2.43 g (12.9 mmol, 99%) of the title compound is isolated as a colorless oil.

¹H-NMR (CDCl₃): δ=0.87 (3H), 0.90 (3H), 1.35 (1H), 1.37 (3H), 1.43 (3H), 1.77 (1H), 2.93 (1H), 3.36 (1H), 3.53 (1H), 3.79 (1H), 3.87 (1H), 3.96 (1H) ppm.

### EXAMPLE 1k

(4S)-4-(2-Methyl-1-oxo-prop-2-yl)-2,2-dimethyl-[1,3]dioxane

The solution of 0.13 ml of oxalyl chloride in 5.7 ml of anhydrous dichloromethane is cooled under an atmosphere of dry argon to −70° C., mixed with 0.21 ml of dimethyl sulfoxide, the solution of 200 mg (1.06 mmol) of the compound, presented according to Example 1i, in 5.7 ml of anhydrous dichloromethane, and it is stirred for 0.5 hour. Then, it is mixed with 0.65 ml of triethylamine, allowed to react for 1 hour at −30° C. and mixed with n-hexane and saturated sodium bicarbonate solution. The organic phase is separated, the aqueous phase is extracted once more with n-hexane, the combined organic extracts are washed with water and dried on magnesium sulfate. The residue that is obtained after filtration and removal of the solvent is further reacted without purification.

### EXAMPLE 1l

(4S)-4-((3RS)-2-methyl-3-hydroxy-pent-2-yl)-2,2-dimethyl-[1,3] dioxane

The solution of 900 mg (4.83 mmol) of the compound, presented according to Example 1k, in 14 ml of anhydrous diethyl ether is mixed with 2.42 ml of a 2.4 molar solution of ethylmagnesium bromide in diethyl ether under an atmosphere of dry argon at 0° C., allowed to heat to 23° C. and stirred for 16 hours. It is mixed with saturated ammonium chloride solution, the organic phase is separated and dried on sodium sulfate. The residue that is obtained after filtration and removal of the solvent is purified by chromatography on fine silica gel with a gradient system that consists of n-hexane and ethyl acetate. 863 mg (3.99 mmol, 83%) of the chromatographically separable 3R- and 3S-epimers of the title compound as well as 77 mg of the title compound that is described in Example 1i are isolated in each case as a colorless oil.

¹H-NMR (CDCl₃) nonpolar isomer: δ=0.86 (3H), 0.89 (3H), 1.03 (3H), 1.25-1.37 (2H), 1.37 (3H), 1.46 (3H), 1.49 (1H), 1.84 (1H), 3.35 (1H), 3.55 (1H), 3.81-4.02 (3H) ppm.

¹H-NMR (CDCl₃) polar isomer: δ=0.72 (3H), 0.91 (3H), 0.99 (3H), 1.25-1.44 (2H), 1.38 (3H), 1.43-1.60 (1H), 1.49 (3H), 1.76 (1H), 3.39 (1H), 3.63 (1H), 3.79-4.03 (3H) ppm.

### EXAMPLE 1m

(4S)-4-(2-Methyl-3-oxo-pent-2-yl)-2,2-dimethyl-[1,3]dioxane

The solution of 850 mg (3.93 mmol) of a mixture of the compound, presented according to Example 1l, in 63 ml of anhydrous dichloromethane is mixed with a molecular sieve (4A, about 80 spheres), 690 mg of n-methylmorpholino-N-oxide, and 70 mg of tetrapropylammonium perruthenate, and it is stirred for 16 hours at 23° C. under an atmosphere of dry argon. It is concentrated by evaporation, and the crude product that is obtained is purified by chromatography on about 200 ml of fine silica gel with a gradient system that consists of n-hexane and ethyl acetate. 728 mg (3.39 mmol, 86%) of the title compound is isolated as a colorless oil.

¹H-NMR(CDCl₃): δ=1.00 (3H), 1.07 (3H), 1.11 (3H), 1.31 (3H), 1.32 (3H), 1.41 (3H), 1.62 (1H), 2.52 (2H), 3.86 (1H), 3.97 (1H), 4.05 (1H) ppm.

EXAMPLE 1n

(S)-Dihydro-3-hydroxy-2(3H)-furanone

10 g of L-(−)-malic acid is stirred in 45 ml of trifluoroacetic acid anhydride for 2 hours at 25° C. Then, it is concentrated by evaporation in a vacuum, 7 ml of methanol is added to the residue, and it is allowed to stir for 12 more hours. Then, it is concentrated by evaporation in a vacuum. The residue that is obtained is dissolved in 150 ml of absolute tetrahydrofuran. It is cooled to 0° C., and 150 ml of borane-tetrahydrofuran complex is added and allowed to stir for 2.5 more hours at 0° C. Then, 150 ml of methanol is added. It is allowed to stir for one more hour at room temperature and then concentrated by evaporation in a vacuum. The crude product that is obtained is dissolved in 80 ml of toluene. 5 g of Dowex (activated, acidic) is added, and it is refluxed for one hour. Then, Dowex is filtered off, and the filtrate is concentrated by evaporation in a vacuum. The crude product that is obtained (7.61 g) is used without purification in the next step.

### EXAMPLE 1o

(S)-Dihydro-3-[[(1,1-dimethylethyl)diphenylsilyl] oxy]-2-(3H)-furanone

24 ml of tert-butyldiphenylsilyl chloride is added to a solution of 7.61 g of the substance that is described under Example in and 10 g of imidazole in 100 ml of N,N-dimethylformamide. It is allowed to stir for two more hours at 25° C., and then the reaction mixture is poured onto ice-cold saturated sodium bicarbonate solution. It is extracted with ethyl acetate, the organic phase is washed with saturated sodium chloride solution, dried on sodium sulfate and concentrated by evaporation in a vacuum. After column chromatography of the crude product on silica gel with a mixture that consists of hexane/ethyl acetate, 13.4 g of the title compound is obtained.

¹H-NMR (CDCl₃): δ=7.72 (2H), 7.70 (2H), 7.40-7.50 (6H), 4.30-4.42 (2H), 4.01 (1H), 2.10-2.30 (2H), 1.11 (9H) ppm.

### EXAMPLE 1p

2RS,3S)-3-[[(1,1-Dimethylethyl)diphenylsilyl]oxy]tetrahydro-2-furanol

80 ml of a 1 molar solution of diisobutylaluminum hydride in hexane is added at −78° C. to a solution of 13.4 g of the substance, described under Example 1o, in 150 ml of absolute tetrahydrofuran. It is stirred for 45 more minutes at −78° C. and then quenched with water. It is extracted with ethyl acetate, the organic phase is washed with saturated sodium chloride solution, dried on sodium sulfate and concentrated by evaporation in a vacuum. 13.46 g of the title compound, which is used without purification in the next step, is obtained.

### EXAMPLE 1q

(2RS,3S)-3-[[(1,1-Dimethylethyl)diphenylsilyl] oxy]-1,4-pentanediol

A solution of 13.46 g of the substance, described under Example 1p, in 150 ml of absolute tetrahydrofuran is added in drops to 20 ml of a 3 molar solution of methylmagnesium chloride in tetrahydrofuran at 0° C. It is allowed to stir for one more hour at 0° C. and then poured onto saturated aqueous ammonium chloride solution. It is extracted with ethyl acetate, the organic phase is washed with saturated sodium chloride solution, dried on sodium sulfate and concentrated by evaporation in a vacuum. After column chromatography of the crude product on silica gel with a mixture of hexane/ethyl acetate, 11.42 g of the title compound is obtained.

¹H-NMR (CDCl₃): δ=7.65-7.75 (4H), 7.40-7.55 (6H), 5.20 (1H), 4.30 (2H), 3.70 (1H), 1.80 (2H), 1.05 (9H) ppm.

### EXAMPLE 1r

(2RS,3S)-5-[[Dimethyl(1,1-dimethylethyl)silyl]oxy]-3-[[(1,1-dimethylethyl)diphenylsilyl] oxy]-2-pentanol

4.9 g of tert-butyldimethylsilyl chloride is added to a solution of 11.42 g of the substance that is described under Example 1q, and 3.25 g of 1H-imidazole in 120 ml of N,N-dimethylformamide. It is allowed to stir for 2 more hours at 25° C., and then the reaction mixture is poured onto ice-cold, saturated sodium bicarbonate solution. It is extracted with ethyl acetate, the organic phase is washed with saturated sodium chloride solution, dried on sodium sulfate and concentrated by evaporation in a vacuum. After column chromatography of the crude product on silica gel with a mixture that consists of hexane/ethyl acetate, 10.64 g of the title compound is obtained.

¹H-NMR (CDCl₃): δ=7.60-7.70 (4H), 7.30-7.45 (6H), 3.70-3.80 (2H), 3.40 (1H), 3.00 (1H), 1.80 (1H), 1.60 (1H), 1.05-1.12 (12H), 0.82 (9H), 0.02 (6H) ppm.

### EXAMPLE 1s

(3S)-5-[[Dimethyl(1,1-dimethylethyl)silyl] oxy]-3-[[(1,1-dimethylethyl)diphenylsilyl] oxy]-2-pentanone

13 ml of dimethyl sulfoxide is added to 7.37 ml of oxalyl chloride in 80 ml of dichloromethane at −78° C. It is allowed to stir for 3 more minutes, and then 10.46 g of the substance, described under Example 1r, in 100 ml of dichloromethane, is added. After another 15 minutes of stirring time, 52 ml of triethylamine is added in drops. Then, it is allowed to heat to 0° C. Then, the reaction mixture is poured onto saturated sodium bicarbonate solution. It is extracted with dichloromethane, the organic phase is washed with saturated sodium chloride solution, dried on sodium sulfate and concentrated by evaporation in a vacuum. After column chromatography of the crude product on silica gel with a mixture that consists of hexane/ethyl acetate, 9.3 g of the title compound is obtained.

¹H-NMR (CDCl₃): δ=7.60-7.70 (4H), 7.32-7.50 (6H), 4.25 (1H), 3.72 (1H), 3.58 (1H), 2.05 (3H), 1.90 (1H), 1.75 (1H), 1.13 (9H), 0.89 (9H), 0.01 (6H) ppm.

### EXAMPLE 1t

(E,3S)-1-[[Dimethyl(1,1-dimethylethyl)silyl]oxy]-3-[[(1,1-dimethylethyl)diphenylsilyl] oxy]-4-methyl-5-(2-methylthiazol-4-yl)-pent-4-ene

The solution of 6.82 g of diethyl(2-methylthiazol-4-yl)methanephosphonate in 300 ml of anhydrous tetrahydrofuran is cooled under an atmosphere of dry argon to −5° C., mixed with 16.2 ml of a 1.6 molar solution of n-butyllithium in n-hexane, allowed to heat to 23° C. and stirred for 2 hours. Then, it is cooled to −78° C., the solution of 6.44 g (13.68 mmol) of the compound, presented according to Example is, in 150 ml of tetrahydrofuran is added in drops, allowed to heat to 23° C. and stirred for 16 hours. It is poured into saturated ammonium chloride solution, extracted several times with ethyl acetate, the combined organic extracts are washed with saturated sodium chloride solution and dried on sodium sulfate. The residue that is obtained after filtration and removal of the solvent is purified by chromatography on fine silica gel with a gradient system that consists of n-hexane and ethyl acetate. 6.46 g (11.4 mmol, 83%) of the title compound is isolated as a colorless oil.

¹H-NMR (CDCl₃): δ=−0.04 (6H), 0.83 (9H), 1.10 (9H), 1.79 (1H), 1.90 (1H), 1.97 (3H), 2.51 (3H), 3.51 (2H), 4.38 (1H), 6.22 (1H), 6.74 (1H), 7.23-7.47 (6H), 7.63 (2H), 7.70 (2H) ppm.

### EXAMPLE 1u

(E,3S)-3-[[(1,1-Dimethylethyl)diphenylsilyl] oxy]-4-methyl-5-(2-methylthiazol-4-yl)-pent-4-en-1-ol

The solution of 4.79 g (8.46 mmol) of the compound, presented according to Example 1t, in 48 ml of tetrahydrofuran is mixed with 48 ml of a 65:35:10 mixture that consists of glacial acetic acid/water/tetrahydrofuran, and it is stirred for 2.5 days at 23° C. It is poured into saturated sodium carbonate solution, extracted several times with ethyl acetate, the combined organic extracts are washed with saturated sodium chloride solution and dried on sodium sulfate. The residue that is obtained after filtration and removal of the solvent is purified by chromatography on fine silica gel with a gradient system that consists of n-hexane and ethyl acetate. 3.42 g (7.57 mmol, 90%) of the title compound is isolated as a colorless oil.

¹H-NMR (CDCl₃): δ=1.10 (9H), 1.53 (1H), 1.81 (2H), 1.96 (3H), 2.71 (3H), 3.59 (2H), 4.41 (1H), 6.38 (1H), 6.78 (1H), 7.26-7.49 (6H), 7.65 (2H), 7.72 (2H) ppm.

### EXAMPLE 1v

(E,3S)-3-[[(1,1-Dimethylethyl)diphenylsilyl]oxy]-4-methyl-5-(2-methylthiazol-4-yl)-pent4-enal

First 4.6 ml of triethylamine, followed by 2.11 g of sulfur trioxide-pyridine complex, are added to a solution of 3.0 g (6.64 mmol) of the compound, presented according to Example 1u, in 65 ml of methylene chloride and 22 ml of DMSO at 23° C. under nitrogen. After one hour of stirring, it is mixed with 20 ml of saturated ammonium chloride solution, stirred for 5 minutes and then diluted with 300 ml of ether. After phase separation, the organic phase is washed twice with 50 ml each of semi-saturated sodium chloride solution. After drying on sodium sulfate and filtration, it is concentrated by evaporation in a vacuum. The thus obtained residue is purified by column chromatography on silica gel. With hexane/0-10% ethyl acetate, 1.27 g of the title compound is obtained as a colorless oil.

¹H-NMR (CDCl₃): δ=1.08 (9H), 1.99 (3H), 2.50 (1H), 2.66 (1H), 2.70 (3H), 4.70 (1H), 6.43 (1H), 6.80 (1H), 7.26-7.49 (6H), 7.5-7.73 (4H) ppm.

### EXAMPLE 1w

(2E,6E,S)-5-[[(1,1-Dimethylethyl)diphenylsilyl] oxy]-2-fluoro-6-methyl-7-(2-methylthiazol-4-yl)-hepta-2,6-dien-1-ol

1.20 g of potassium carbonate is added to a solution of 2.10 g (8.67 mmol) of triethyl-2-fluoro-2-phosphonoacetate in 5 ml of ethanol, and it is stirred for 10 minutes at 23° C. under nitrogen. Then, a solution of 3.0 g (6.67 mmol) of the title compound, presented under lv, in 3 ml of ethanol is added in drops, and it is stirred for 3 hours. It is diluted with 300 ml of ether and washed twice with 30 ml of semi-saturated sodium chloride solution. After drying on sodium sulfate and filtration, it is concentrated by evaporation in a vacuum. The thus obtained residue is purified by column chromatography on silica gel. With hexane/0-10% ethyl acetate,3.32 g of (2E/Z,6E,3S)-5-[[(1,1-dimethylethyl)-diphenylsilyl]oxy]-2-fluoro-6-methyl-7-(2-methylthiazol-4-yl)-hepta-2,6-dienoic acid ethyl ester is obtained as a colorless oil.

16 ml of a 1.2 molar solution of DIBAH in toluene is added to a solution of 3.30 g (6.30 mmol) of the thus obtained ester in 26 ml of toluene at −70° C. under nitrogen. It is allowed to heat within 2 hours to 0° C. and then cooled again to −70° C. Then, 3 ml of isopropanol, followed by 8 ml of water, are carefully added in drops to the reaction mixture, allowed to heat to 23° C. and stirred for another 2 hours. Precipitate is filtered out, it is thoroughly rewashed with ethyl acetate and concentrated by evaporation in a vacuum. The thus obtained residue is purified by 2×column chromatography on silica gel. With hexane/0-20% ethyl acetate, 1.14 of the Z-isomeric alcohol is obtained as a polar fraction, and 1.47 g of the title compound is obtained as a colorless oil.

Title compound, nonpolar fraction: ¹H-NMR (CDCl₃): δ=1.08 (9H), 1.90 (3H), 2.22 (2H), 2.68 (3H), 3.96 (1H), 4.03 (1H), 4.17 (1H), 5.01 (1H), 6.25 (1H), 6.77 (1H), 7.26-7.49 (6H), 7.55-7.74 (4H) ppm.

Z-isomers, polar fraction: ¹H-NMR (CDCl₃): δ=1.08 (9H), 1.91 (3H), 2.36 (2H), 2.69 (3H), 3.95 (2H), 4.24 (1H), 4.66 (1H), 6.29 (1H), 6.78 (1H), 7.22-7.48 (6H), 7.58-7.74 (4H) ppm.

### EXAMPLE 1x

(6E,10E,2S,9S)-9-[[(1,1-Dimethylethyl)diphenylsilyl]oxy]-6-fluoro-2,10-dimethyl-11-(2-methylthiazol-4-yl)-1-(tetrahydro-2H-pyran-2-yloxy)-4-oxa-6,10-undecadiene

A mixture that consists of 1.45 g (2.93 mmol) of the title compound that was produced under lw and 2.77 g (11.7 mmol) of (R)-2-methyl-3-tetrahydropyranyloxypropyl bromide (K. Mori et al. Tetrahedron 41; 541-546 (1985)) in 4.5 ml of 50% sodium hydroxide solution and 1 ml of toluene is stirred vigorously for 18 hours. It is diluted with 400 ml of ether, washed three times with 30 ml each of semi-saturated sodium chloride solution, dried on sodium sulfate and concentrated by evaporation in a vacuum after filtration. The thus obtained residue is purified by column chromatography on silica gel. With hexane/0-16% ethyl acetate, 1.63 g of the title compound is obtained as a colorless oil.

¹H-NMR (CDCl₃): δ=0.91 (3H), 1.07 (9H), 1.4-2.05 (7H), 1.98 (3H), 2.23 (2H), 2.70 (3H), 3.10-3.70 (5H), 3.80 (2H), 3.75-3.90 (1H), 4.16 (1H), 4.55 (1H), 5.09 (1H), 6.28 (1H), 6.80 (1H), 7.20-7.48 (6H), 7.55-7.73 (4H) ppm.

### EXAMPLE 1y

(6E,10E,2S,9S)-2,10-Dimethyl-6-fluoro-11-(2-methylthiazol-4-yl)-1-(tetrahydro-2H-pyran-2-yloxy)-4-oxa-6,10-undecadien-9-ol

1.32 g of tetrabutylammonium fluoride trihydrate is added to a solution that consists of 1.71 g (2.62 mmol) of the title compound, produced under 1×, in 9 ml of tetrahydrofuran, and it is stirred for 3 hours at 40° C. Then, it is diluted with 150 ml of ether, washed once with 20 ml of water, twice with 20 ml each of semi-saturated sodium chloride solution, dried on sodium sulfate and concentrated by evaporation in a vacuum after filtration. The thus obtained residue is purified by column chromatography on silica gel. With hexane/0-20% ethyl acetate, 655 mg of the title compound is obtained as a colorless oil.

¹H-NMR (CDCl₃): δ=0.97 (3H), 1.42-2.15 (7H), 2.05 (3H), 2.25-2.46 (3H), 2.71 (3H), 3.24-3.57 (4H), 3.65 (1H), 3.84 (1H), 4.08 (2H), 4.16 (1H), 4.56 (1H), 5.32 (1H), 6.56 (1H), 6.95 (1H) ppm.

### EXAMPLE 1z

(6E,10E,2S,9S)-9-[[(1,1-Dimethylethyl)dimethylsilyl] oxy]-6-fluoro-2,10-dimethyl-11-(2-methylthiazol-4-yl)-4-oxa-6,10-undecadien-1-ol

392 mg of imidazole and 0.62 ml of a 4.64 M solution of tert-butyldimethylsilyl chloride in hexane are added to a solution of 650 mg (1.57 mmol) of the title compound, produced under ly, in 8 ml of DMF. After 4 hours of stirring at 23° C., it is diluted with 150 ml of a mixture that consists of ether and hexane at a ratio of 1:1. Then, the organic phase is washed once with 20 ml of water, twice with 20 ml each of semi-saturated sodium chloride solution, dried on sodium sulfate and concentrated by evaporation in a vacuum after filtration. The thus obtained residue is purified by column chromatography on silica gel. With hexane/0-10% ethyl acetate, 740 mg of silyl ether is obtained as a colorless oil.

113 mg of Amberlyst 15 is added to a solution of 734 mg of this silyl ether in 6 ml of methanol, and it is stirred for 3 hours at 23° C. under nitrogen. After filtration, it is concentrated by evaporation in a vacuum. The thus obtained residue is purified by column chromatography on silica gel. With hexane/0-15% ethyl acetate, 277 mg of the title compound is obtained as a colorless oil.

¹H-NMR (CDCl₃): δ=0.01 (3H), 0.06 (3H), 0.87 (3H), 0.89 (9H), 1.94-2.05 (1H), 2.00 (3H), 2.28 (2H), 2.49 (1H), 2.71 (3H), 3.40 (1H), 3.51 (1H), 3.58 (2H), 3.95-4.20 (3H), 5.32 (1H), 6.47 (1H), 6.94 (1H) ppm.

### EXAMPLE 1aa

(4S(4R,5S,6S,10E,14E))-{13-[[(1,1-Dimethylethyl)dimethylsilyl] oxy]-15-(2-methylthiazol-4-yl)-8-oxa-3-oxo-5-hydroxy-10-fluoro-2,4,6,14-tetramethyl-pentadeca-10,14-dien-2-yl}-2,2-dimethyl-[1,3]dioxane

0.43 ml of triethylamine is added in drops to a solution of 272 mg (0.61 mmol) of the alcohol, produced under 1z, in a mixture that consists of 6 ml of methylene chloride and 2 ml of DMSO. Then, 195 mg of sulfur trioxide-pyridine complex is added, and it is stirred for 1 hour at 23° C. under nitrogen. Then, 5 ml of ammonium chloride solution is added, and it is diluted after 5 minutes with 150 ml of ether. The organic phase is washed twice with 20 ml each of semi-saturated sodium chloride solution, dried on sodium sulfate and concentrated by evaporation in a vacuum after filtration. The thus obtained crude product (267 mg) is used without further purification in the next step.

1.33 ml of a 1.6 molar solution of butyllithium in hexane is added to a solution of 221 mg of diisopropylamine in 3.8 ml of tetrahydrofuran at 0° C. under nitrogen. After 15 minutes of stirring, it is cooled to −70° C., and a solution of 390 mg of the compound, produced according to Example 1m, in 3.8 ml of tetrahydrofuran is added in drops. After 1.25 hours of stirring, 267 mg of the above-produced aldehyde in 1.3 ml of tetrahydrofuran is added in drops. After 1 hour of stirring at this temperature, 5 ml of a saturated ammonium chloride solution is added in drops and diluted after 5 minutes with 150 ml of ethyl acetate. The organic phase is washed twice with 20 ml each of semi-saturated sodium chloride solution, dried on sodium sulfate, and concentrated by evaporation in a vacuum after filtration. The thus obtained residue is purified by chromatography on silica gel. With hexane/0-40% ether, 113 mg of the title compound as a nonpolar fraction is obtained as a colorless oil. In addition, 131 mg of a mixed fraction and 29 mg of the diastereomeric aldol product are obtained. The mixed fraction is chromatographed repeatedly. As a result, another 40 mg of the desired title compound is obtained.

¹H-NMR (CDCl₃): δ=0.00 (3H), 0.05 (3H), 0.89 (9H), 0.94 (3H), 1.04 (3H), 1.08 (3H), 1.21 (3H), 1.32 (3H), 1.39 (3H), 1.0-1.42 (1H), 1.54-1.72 (1H), 1.80 (1H), 1.99 (3H), 2.17-2.36 (2H), 2.71 (3H), 3.23 (1H), 3.42-3.69 (4H), 3.80-4.19 (6H), 5.28 (1H), 6.50 (1H), 6.93 (1H) ppm.

### EXAMPLE 1ab

(3S,6R,7S,8S,12E,16E)-15-[[(1,1-Dimethylethyl)dimethylsilyl] oxy]-10-oxa-5-oxo-17-(2-methylthiazol-4-yl)-12-fluoro-4,4,6,8,16-pentamethyl-heptadeca-12,16-diene-1,3,7-triol

44 mg of p-toluenesulfonic acid is added to a solution of 151 mg (0.23 mmol) of the title compound, produced in laa, in 10.8 ml of ethanol, and it is stirred for 3 hours at 23° C. Then, it is diluted with 80 ml of ethyl acetate, and the organic phase is washed once with 10 ml of saturated sodium bicarbonate solution and three times with 10 ml each of semi-saturated sodium chloride solution, and it is dried on sodium sulfate. After filtration, it is concentrated by evaporation in a vacuum, and the thus obtained residue is purified by chromatography on silica gel. With hexane/0-40% ethyl acetate, 125 mg of the title compound is obtained as a colorless oil.

¹H-NMR (CDCl₃): δ=0.00 (3H), 0.05 (3H), 0.89 (9H), 0.95 (3H), 1.08 (3H), 1.11 (3H), 1.24 (3H), 1.0-1.91 (3H), 1.98 (3H), 2.27 (2H), 2.71 (3H), 2.86 (1H), 3.24 (1H), 3.38-3.70 (6H), 3.88 (2H), 3.97-4.18 (3H), 5.30 (1H), 6.45 (1H), 6.94 (1H) ppm.

### EXAMPLE 1ac

(3S,6R,7S,8S,12E,16E)-1,3,7,15-Tetrakis [[(1,1-dimethylethyl)dimethylsilyl]oxy]-10-oxa-5-oxo-17-(2-methylthiazol-4-yl)-12-fluoro-4,4,6,8,16-pentamethyl-heptadeca-12,16-diene

226 mg of 2,6-lutidine and 319 mg of trifluoromethanesulfonic acid-tert-butyldimethylsilyl ester are added to a solution of 123 mg of the title compound, produced in Example lab, in 6.8 ml of methylene chloride, and it is stirred for 16 hours at −20° C. Then, 5 ml of saturated ammonium chloride solution is added, diluted with 80 ml of ethyl acetate, and after phase separation, the organic phase is washed once with 10 ml of water and twice with semi-saturated sodium chloride solution. It is dried on sodium sulfate and concentrated by evaporation in a vacuum after filtration. The thus obtained residue is purified by chromatography on silica gel. With hexane/0-10% ethyl acetate, 166 mg of the title compound is obtained as a colorless oil.

¹H-NMR (CDCl₃): δ=0.00-0.10 (24H), 0.85-0.90 (36H), 0.95 (3H), 1.03 (3H), 1.05 (3H), 1.21 (3H), 1.1-1.75 (3H), 2.00 (3H), 2.27 (2H), 2.71 (3H), 3.18 (1H), 3.31 (1H), 3.52-3.75 (3H), 3.80-4.16 (5H), 5.28 (1H), 6.46 (1H), 6.92 (1H) ppm.

### EXAMPLE 1ad

(3S,6R,7S,8S,12E,16E)-3,7,15-Tris [[(1,1-dimethylethyl)dimethylsilyl]oxy]-10-oxa-5-oxo-17-(2-methylthiazol-4-yl)-12-fluoro-4,4,6,8,16-pentamethyl-heptadeca-12,16-dien-1-ol

40 mg of camphor-10-sulfonic acid is added to a solution of 165 mg of the title compound, produced under 1 ac, in 2.7 ml of a 1:1 mixture of methylene chloride and methanol at 0° C., and it is stirred for 3.5 hours at this temperature. After adding 0.5 ml of triethylamine, it is stirred for 5 minutes and then added to 20 ml of saturated sodium bicarbonate solution. It is extracted three times with 30 ml each of methylene chloride and then the combined organic phases are washed twice with 10 ml each of semi-saturated sodium chloride solution, dried on sodium sulfate and concentrated by evaporation in a vacuum after filtration. The thus obtained residue is purified by chromatography on silica gel. With hexane/0-10% ethyl acetate, 134 mg of the title compound is obtained as a colorless oil.

¹H-NMR (CDCl₃): δ=0.00-0.10 (18H), 0.85-0.92 (27H), 0.95 (3H), 1.04 (3H), 1.11 (3H), 1.19 (3H), 1.69 (2H), 1.91 (1H), 2.00 (3H), 2.27 (2H), 2.71 (3H), 3.19 (1H), 3.35 (1H), 3.56-3.71 (3H), 3.85 (1H), 3.91-4.16 (4H), 5.28 (1H), 6.46 (1H), 6.92 (1H) ppm.

### EXAMPLE 1ae

(3S,6R,7S,8S,12E,16E)-3,7,15-Tris [[(1,1-dimethylethyl)dimethylsilyl]oxy]-10-oxa-5-oxo-17-(2-methylthiazol-4-yl)-12-fluoro-4,4,6,8,16-pentamethyl-heptadeca-12,16-dienoic acid

50 mg of sulfur trioxide-pyridine complex is added to a solution of 133 mg of the title compound, produced under 1ad, in a mixture of 1.7 ml of methylene chloride and 0.4 ml of dimethyl sulfoxide at 23° C., and it is stirred for 1 hour at this temperature. Then, 2 ml of saturated ammonium chloride solution is added, and it is diluted with 80 ml of ether. The organic phase is washed twice with 10 ml each of semi-saturated sodium chloride solution, dried on sodium sulfate, and concentrated by evaporation in a vacuum after filtration. The thus obtained crude product is used without further purification in the next step.

1.5 ml of water, 133 mg of sodium chlorite (80%) and 79 mg of sodium dihydrogen phosphate-monohydrate are added to the above-produced aldehyde in a solution of 5.4 ml of a 2 molar solution of 2-methyl-2-butene in tetrahydrofuran and 7.2 ml of tert-butanol at 0° C. while being stirred vigorously, and it is stirred for 3 hours at this temperature. Then, the reaction mixture is added to 10 ml of saturated sodium thiosuflate solution and extracted three times with 30 ml each of ethyl acetate. The combined organic phases are washed twice with 10 ml each of semi-saturated sodium chloride solution, dried on sodium sulfate and concentrated by evaporation in a vacuum after filtration. The thus obtained residue is purified by chromatography on silica gel. With hexane/0-10% ethyl acetate, 110 mg of the title compound is obtained as a colorless oil.

¹H-NMR (CDCl₃): δ=0.00-0.12 (18H), 0.82-0.91 (27H), 0.95 (3H), 1.06 (3H), 1.17 (3H), 1.18 (3H), 1.75 (1H), 1.95 (3H), 2.15-2.53 (4H), 2.72 (3H), 3.22 (1H), 3.32 (1H), 3.58 (1H), 3.84 (1H), 3.93-4.19 (3H), 4.42 (1H), 5.31 (1H), 6.60 (1H), 6.94 (1H) ppm.

### EXAMPLE 1af

(3S,6R,7S,8S,12E,16E)-3,7-Bis [[(1,1-dimethylethyl)dimethylsilyl] oxy]-10-oxa-5-oxo-17-(2-methylthiazol-4-yl)-12-fluoro-15-hydroxy-4,4,6,8,16-pentamethyl-heptadeca-12,16-dienoic Acid

238 mg of tetrabutylammonium fluoride trihydrate is added to a solution of 108 mg of the title compound, produced under lae, in 2.2 ml of tetrahydrofuran at 23° C., and it is stirred for 2 hours at this temperature. Then, the reaction mixture is diluted with 80 ml of ethyl acetate and washed three times with 10 ml each of semi-saturated sodium chloride solution, dried on sodium sulfate and concentrated by evaporation in a vacuum after filtration. The thus obtained residue is purified by chromatography on silica gel. With hexane/0-50% ethyl acetate, 46 mg of the title compound, in addition to 30 mg of starting material, is obtained as a colorless oil.

¹H-NMR (CDCl₃): δ=0.07 (9H), 0.10 (3H), 0.88 (9H), 0.90 (9H), 0.99 (3H), 1.07 (3H), 1.17 (3H), 1.26 (3H), 2.00 (3H), 2.21-2.56 (4H), 2.72 (3H), 3.18-3.35 (2H), 3.61 (1H), 3.86 (1H), 3.96-4.20 (4H), 4.43 (1H), 5.32 (1H), 6.70 (1H), 6.96 (1H) ppm.

### EXAMPLE 1ag

(4S,7R,8S,9S,13E,16S(E))-4,8-Bis [[(1,1-dimethylethyl)dimethylsilyl]oxy]-13-fluoro-16-(1-methyl-2-(2-methyl-4-thiazolyl)ethenyl)-1,l 1-dioxa-5,5,7,9-tetramethyl-cyclohexadec-13-ene-2,6-dione

0.022 ml of triethylamine, followed by 0.014 ml of 2,4,6-trichlorobenzoyl chloride are added to a solution of 52 mg of the title compound, produced under 1af, in 1.0 ml of tetrahydrofuran at 0° C., and it is stirred for 1 hour at this temperature. Then, this mixture is added by means of a metering pump within 3 hours to a solution of 86 mg of p-N,N-dimethylaminopyridine in 33 ml of toluene, and it is stirred for 18 hours at 23° C. The reaction mixture is concentrated by evaporation in a vacuum, and the thus obtained residue is purified by chromatography on silica gel. With hexane/0-30% ethyl acetate, 28 mg of the title compound is obtained as a colorless oil.

¹H-NMR (CDCl₃): δ=−0.07 (3H), 0.06 (3H), 0.09 (3H), 0.11 (3H), 0.83 (9H), 0.93 (9H), 1.06 (3H), 1.09 (3H), 1.15 (3H), 1.21 (3H), 1.94 (1H), 2.11 (3H), 2.11-2.54 (2H), 2.71 (3H), 2.77-3.26 (4H), 3.65-4.28 (5H), 5.05 (1H), 5.19 (1H), 6.57 (1H), 6.98 (1H) ppm.

### EXAMPLE 1

(4S,7R,8S,9S,13E,16S(E))-4,8-Dihydroxy-13-fluoro-16-(1-methyl-2-(2-methyl-4-thiazolyl)ethenyl)-1,11-dioxa-5,5,7,9-tetramethyl-cyclohexadec-13-ene-2,6-dione

0.24 ml of HF/pyridine is added to a solution of 24 mg of the title compound, produced under lag, in 2.5 ml of tetrahydrofuran at 230C, and it is stirred for 2 hours at this temperature. After adding another amount of 0.24 ml of HF/pyridine, it is then stirred for another 18 hours at this temperature. Then, this mixture is added to 10 ml of saturated sodium bicarbonate solution and diluted with 30 ml of water. It is extracted three times with 30 ml of ethyl acetate each. The combined organic phases are washed once with 10 ml of semi-saturated sodium chloride solution, dried on sodium sulfate and concentrated by evaporation in a vacuum after filtration. The thus obtained residue is purified by a preparative thick-layer chromatography. With hexane/50% ethyl acetate, 12.8 mg of the title compound is obtained as a colorless oil.

¹H-NMR (CDCl₃): δ=1.04 (3H), 1.06 (3H), 1.13 (3H), 1.36 (3H), 1.85 (1H), 2.06 (3H), 2.22-2.49 (3H), 2.69 (3H), 2.68-2.86 (1H), 3.29 (2H), 3.44 (1H), 3.82 (1H), 3.91 (1H), 3.99 (1H), 4.23 (1H), 4.46 (1H), 5.13-5.33 (2H), 6.59 (1H), 6.95 (1H) ppm.

### EXAMPLE 2

(4S,7R,8S,9S,13E,16S(E))-4,8-Dihydroxy-13-chloro-16-(1-methyl-2-(2-methyl-4-thiazolyl)ethenyl)-1,11-dioxa-5,5,7,9-tetramethyl-cyclohexadec-13-ene-2,6-dione

### EXAMPLE 2a

(2E,6E,S)-5-[[(1,1-Dimethylethyl)diphenylsilyl]oxy]-2-chloro-6-methyl-7-(2-methylthiazol-4-yl)-hepta-2,6-dien-1-ol

30 ml of a 0.5 molar solution of potassium-bis-(trimethylsilyl)-amide in toluene is added to a solution of 3.88 g (15.0 mmol) of triethyl-2-chloro-2-phosphonoacetate and 19.8 g of 18-crown-6 in 280 ml of tetrahydrofuran at −70° C. After 30 minutes of stirring, 4.5 g (10.0 mmol) of the aldehyde, produced under lv, in 45 ml of tetrahydrofuran is added in drops and stirred for 1 hour at −70° C. The reaction mixture is added to 50 ml of saturated ammonium chloride solution and extracted three times with 300 ml of ether each. The combined organic phases are washed twice with 50 ml each of semi-saturated sodium chloride solution, dried on sodium sulfate, and concentrated by evaporation in a vacuum after filtration. The thus obtained residue is purified by column chromatography on silica gel. With hexane/0-15% ethyl acetate, 4.92 g of (2E/Z,6E,3S)-5-[[(1,1-dimethylethyl)diphenylsilyl]oxy]-2-chloro-6-methyl-7-(2-methylthiazol-4-yl)-hepta-2,6-dienoic acid ethyl ester is obtained as a colorless oil.

22 ml of a 1.2 molar solution of DIBAH in toluene is added to a solution of 4.92 g (8.88 mmol) of the thus obtained ester in 37 ml of toluene at −70° C. under nitrogen. It is allowed to heat to 0° C. within 2 hours and then cooled again to −70° C. Then, 2 ml of isopropanol, followed by 11 ml of water, are carefully added in drops to the reaction mixture, allowed to heat to 23° C. and stirred for another two hours. Precipitate is filtered out, it is thoroughly rewashed with ethyl acetate and concentrated by evaporation in a vacuum. The thus obtained residue is purified by column chromatography on silica gel. With hexane/0-20% ethyl acetate, 1.31 of the Z-isomeric alcohol is obtained as a polar fraction, and 1.26 g of the title compound is obtained as a colorless oil.

Title compound, nonpolar fraction: ¹H-NMR (CDCl₃): δ=1.08 (9H), 1.91 (3H), 2.34 (2H), 2.69 (3H), 4.01 (1H), 4.10 (1H), 4.19 (1H), 5.60 (1H), 6.26 (1H), 6.77 (1H), 7.26-7.49 (6H), 7.55-7.74 (4H) ppm.

Z-isomer, polar fraction: ¹H-NMR (CDCl₃): δ=1.08 (9H), 1.95 (3H), 2.48 (2H), 2.69 (3H), 4.01 (2H), 4.30 (1H), 5.61 (1H), 6.29 (1H), 6.79 (1H), 7.22-7.48 (6H), 7.58-7.74 (4H) ppm.

### EXAMPLE 2b

(6E,10E,2S,9S)-9-[[(1,1-Dimethylethyl)diphenylsilyl]oxy]-6-chloro-2,10-dimethyl-11-(2-methylthiazol-4-yl)-1-(tetrahydro-2H-pyran-2-yloxy)-4-oxa-6,10-undecadiene

Analogously to Example 1x, 1.03 g of the title compound is obtained from 1.15 g (2.25 mmol) of the alcohol, produced under 2a, as a colorless oil.

¹H-NMR (CDCl₃): δ=0.92 (3H), 1.07 (9H), 1.40-2.09 (7H), 1.98 (3H), 2.24-2.48 (2H), 2.70 (3H), 3.08-3.35 (3H), 3.43-3.69 (2H), 3.83 (1H), 3.89 (2H), 4.18 (1H), 4.55 (1H), 5.69 (1H), 6.28 (1H), 6.80 (1H), 7.22-7.48 (6H), 7.56-7.71 (4H) ppm.

### EXAMPLE 2c

(6E,10E,2S,9S)-2,10-Dimethyl-6-chloro-11-(2-methylthiazol-4-yl)-1-(tetrahydro-2H-pyran-2-yloxy)-4-oxa-6,10-undecadien-9-ol

Analogously to Example 1y, 597 mg of the title compound is obtained from 1.02 g (1.53 mmol) of the compound, produced under 2b, as a colorless oil.

¹H-NMR (CDCl₃): δ=0.98 (3H), 1.42-1.90 (4H), 2.05 (3H), 1.97-2.14 (1H), 2.25 (1H), 2.48 (2H), 2.71 (3H), 3.26-3.56 (4H), 3.66 (1H), 3.84 (1H), 4.08-4.25 (4H), 4.57 (1H), 5.92 (1H), 6.57 (1H), 6.96 (1H) ppm.

### EXAMPLE 2d

(6E,10E,2S,9S)-9-[[(1,1-Dimethylethyl)dimethylsilyl]oxy]-6-chloro-2,10-dimethyl-11-(2-methylthiazol-4-yl)-4-oxa-6,10-undecadien-1-ol

Analogously to Example 1z, 454 mg of the title compound is obtained from 595 mg (1.38 mmol) of the compound, produced under 2c, as a colorless oil.

¹H-NMR (CDCl₃): δ=0.01 (3H), 0.06 (3H), 0.88 (3H), 0.89 (9H), 2.01 (3H), 2.04 (1H), 2.25-2.65 (3H), 2.71 (3H), 3.36 (1H), 3.49 (1H), 3.55-3.70 (2H), 4.08 (1H), 4.15 (1H), 4.21 (1H), 5.91 (1H), 6.48 (1H), 6.94 (1H) ppm.

### EXAMPLE 2e

(4S(4R,5S,6S,10E,14E))-{13-[[(1,1-Dimethylethyl)dimethylsilyl]oxy]-15-(2-methylthiazol4-yl)-8-oxa-3-oxo-5-hydroxy-10-chloro-2,4,6,14-tetramethyl-pentadeca-10,14-dien-2-yl}-2,2-dimethyl-[1,3]dioxane

Analogously to Example 1aa, in addition to 168 mg of starting material, 128 mg of slightly contaminated title compound and 174 mg of a clean fraction of the title compound are obtained from 450 mg (0.98 mmol) of the compound, produced under 2d, as a colorless oil.

¹H-NMR (CDCl₃): δ=0.00 (3H), 0.05 (3H), 0.89 (9H), 0.96 (3H), 1.04 (3H), 1.08 (3H), 1.21 (3H), 1.32 (3H), 1.39 (3H), 1.44-1.72 (2H), 1.79 (1H), 2.00 (3H), 2.19-2.28 (2H), 2.71 (3H), 3.24 (1H), 3.39-3.65 (4H), 3.86 (1H), 3.90-4.23 (5H), 5.87 (1H), 6.47 (1H), 6.94 (1H) ppm.

### EXAMPLE 2f

(3S,6R,7S,8S,12E,16E)-15-[[(1,1-Dimethylethyl)dimethylsilyl]oxy]-10-oxa-5-oxo-17-(2-methylthiazol-4-yl)-12-chloro-4,4,6,8,16-pentamethyl-heptadeca-12,16-diene-1,3,7-triol

Analogously to Example 1ab, 187 mg of the title compound is obtained from 226 mg (0.336 mmol) of the compound, produced under 2e, as a colorless oil.

¹H-NMR (CDCl₃): δ=0.00 (3H), 0.06 (3H), 0.89 (9H), 0.97 (3H), 1.09 (3H), 1.12 (3H), 1.24 (3H), 1.2-1.9 (3H), 1.99 (3H), 2.38 (2H), 2.71 (3H), 2.86 (1H), 3.25 (1H), 3.38-3.70 (6H), 3.87 (2H), 3.97-4.25 (3H), 5.89 (1H), 6.47 (1H), 6.95 (1H) ppm.

### EXAMPLE 2g

(3S,6R,7S,8S,12E,16E)-1,3,7,15-Tetrakis [[(1,1-dimethylethyl)dimethylsilyl]oxy]-10-oxa-5-oxo-17-(2-methylthiazol4-yl)-12-chloro-4,4,6,8,16-pentamethyl-heptadeca-12,16-diene

Analogously to Example 1ac, 252 mg of the title compound is obtained from 186 mg (0.29 mmol) of the title compound, produced under 2f, as a colorless oil.

¹H-NMR (CDCl₃): δ=0.00-0.10 (24H), 0.80-0.90 (36H), 0.97 (3H), 1.03 (3H), 1.06 (3H), 1.22 (3H), 1.35-1.78 (3H), 2.00 (3H), 2.24-2.51 (2H), 2.71 (3H), 3.14 (1H), 3.35 (1H), 3.51-3.73 (3H), 3.82 (1H), 3.89 (1H), 4.03 (1H), 4.12 (1H), 4.15 (1H), 5.87 (1H), 6.48 (1H), 6.93 (1H) ppm.

### EXAMPLE 2h

(3S,6R,7S,8S,12E,16E)-3,7,15-Tris[[(1,1-dimethylethyl)dimethylsilyl]oxy]-10-oxa-5-oxo-17-(2-methylthiazol-4-yl)-12-chloro-4,4,6,8,16-pentamethyl-heptadeca-12,16-dien-1-ol

Analogously to Example 1ad, 204 mg of the title compound is obtained from 248 mg (0.25 mmol) of the compound, produced under 2 g, as a colorless oil.

¹H-NMR (CDCl₃): δ=0.00-0.10 (18H), 0.85-0.92 (27H), 0.96 (3H), 1.05 (3H), 1.13 (3H), 1.21 (3H), 1.2-2.0 (4H), 2.01 (3H), 2.28-2.50 (2H), 2.71 (3H), 3.16 (1H), 3.40 (1H), 3.59 (1H), 3.64 (2H), 3.86 (1H), 4.03 (1H), 4.09 (1H), 4.13 (1H), 4.16 (1H), 5.87 (1H), 6.48 (1H), 6.93 (1H) ppm.

### EXAMPLE 2i

(3S,6R,7S,8S,12E,16E)-3,7,15-Tris[[(1,1-dimethylethyl)dimethylsilyl]oxy]-10-oxa-5-oxo-17-(2-methylthiazol-4-yl)-12-chloro-4,4,6,8,16-pentamethyl-heptadeca-12,16-dienoic Acid

Analogously to Example 1ae, 156 mg of the title compound is obtained from 202 mg (0.235 mmol) of the compound, produced under 2 h, as a colorless oil.

¹H-NMR (CDCl₃): δ=0.00-0.12 (18H), 0.82-0.92 (27H), 0.96 (3H), 1.06 (3H), 1.19 (6H), 1.96 (3H), 2.24-2.52 (4H), 2.72 (3H), 3.18 (1H), 3.35 (1H), 3.56 (1H), 3.85 (1H), 3.94 (1H), 3.97 (1H), 4.10 (1H), 4.09-4.19 (1H), 4.19 (1H), 4.42 (1H), 5.91 (1H), 6.61 (1H), 6.94 (1H) ppm.

### EXAMPLE 2j

(3S,6R,7S,8S,12E,16E)-3,7-Bis[[(1,1-dimethylethyl)dimethylsilyl]oxy]-10-oxa-5-oxo-17-(2-methylthiazol-4-yl)-12-chloro-15-hydroxy-4,4,6,8,16-pentamethyl-heptadeca-12,16-dienoic Acid

Analogously to Example 1af, 108 mg of the title compound is obtained from 155 mg (0.177 mmol) of the compound, produced under 2i, as a colorless oil.

¹H-NMR (CDCl₃): δ=0.00-0.12 (12H), 0.82-0.92 (18H), 0.99 (3H), 1.07 (3H), 1.17 (3H), 1.20 (3H), 2.00 (3H), 1.2-2.52 (6H), 2.72 (3H), 3.18 (1H), 3.30 (1H), 3.58 (1H), 3.85 (1H), 4.10 (1H), 4.10-4.23 (1H), 4.30 (1H), 4.42 (1H), 5.94 (1H), 6.69 (1H), 6.96 (1H) ppm.

### EXAMPLE 2k

(4S,7R,8S,9S,13E,16S(E))-4,8-Bis [[(1,1-dimethylethyl)dimethylsilyl]oxy]-13-chloro-16-(1-methylActive-ingredient-containing(2-methyl4-thiazolyl)ethenyl)-1,11-dioxa-5,5,7,9-tetramethyl-cyclohexadec-13-ene-2,6-dione

Analogously to Example 1ag, 76 mg of the title compound is obtained from 105 mg (0.138 mmol) of the compound, produced under 2j, as a colorless oil.

¹H-NMR (CDCl₃): δ=−0.05 (3H), 0.07 (3H), 0.10 (3H), 0.12 (3H), 0.84 (9H), 0.93 (9H), 1.08 (3H), 1.09 (3H), 1.15 (3H), 1.20 (3H), 1.8-2.4 (2H), 2.11 (3H), 2.71 (3H), 2.75-2.95 (2H), 3.05-3.28 (2H), 3.75 (2H), 3.94 (1H), 4.04 (1H), 4.22 (1H), 4.42 (1H), 5.05 (1H), 5.78 (1H), 6.57 (1H), 6.98 (1H) ppm.

### EXAMPLE 2

(4S,7R,8S,9S,13E,16S(E))-4,8-Dihydroxy-13-chloro-16-(1-methyl-2-(2-methyl-4-thiazolyl)ethenyl)-1,11-dioxa-5,5,7,9-tetramethyl-cyclohexadec-13-ene-2,6-dione

0.22 ml of a 20% trifluoroacetic acid solution in methylene chloride is added to a solution of 32 mg (0.044 mmol) of the compound, produced under 2 k, in 0.22 ml of methylene chloride at −20° C., it is allowed to heat slowly to 0° C. and stirred for another 4 hours. Then, the reaction mixture is concentrated by evaporation in a vacuum, and the thus obtained residue is purified analogously to Example 1. In this way, 10.4 mg of the title compound is obtained as a colorless oil.

¹H-NMR (CDCl₃): δ=1.04 (3H), 1.07 (3H), 1.15 (3H), 1.35 (3H), 1.86 (1H), 2.07 (3H), 2.30 (1H), 2.39 (1H), 2.43 (1H), 2.69 (3H), 3.00 (1H), 3.24 (1H), 3.29 (1H), 3.44 (1H), 3.49 (1H), 3.78 (1H), 3.89 (1H), 4.04 (1H), 4.31 (1H), 4.46 (1H), 5.26 (1H), 5.79 (1H), 6.59 (1H), 6.96 (1H) ppm.

1. Epothilone derivatives of general formula I, 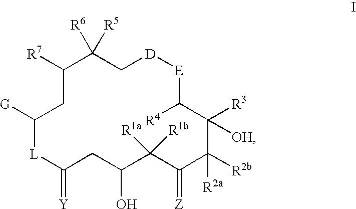
in which
R^{1a}, R^{1b} are the same or different and mean hydrogen, C₁-C₁₀-alkyl, aryl, C₇-C₂₀-aralkyl, or together a —(CH₂)ₘ group with m=2, 3, 4 or 5, or a —CH₂—O—CH₂ group,
R^{2a}, R^{2b} are the same or different and mean hydrogen, C₁-C₁₀-alkyl, aryl, C₇-C₂₀-aralkyl, —(CH₂)ᵣ—C≡C—(CH₂)ₚ—R²⁰, —(CH₂)ₙCH═CH—(CH₂)ₚ—R²⁰,
r are the same or different and mean 0 to 4,
p are the same or different and mean 0 to 3,
R²⁰ is equal to hydrogen, C₁-C₁₀-alkyl, aryl, C₇-C₂₀-aralkyl, C₁-C₁₀-acyl or if p>0, a group OR²¹,
R²¹ means hydrogen, or a protective group PG6,
R³ means hydrogen, C₁-C₁₀-alkyl, aryl, C₇-C₂₀-aralkyl,
R⁴ means hydrogen, C₁-C₁₀-alkyl, aryl, C₇-C₂₀-aralkyl,
D means oxygen, sulfur, sulfoxide or sulfone, whereby then E must be equal to methylene or
D-E together mean a group
H₂C—CH₂, HC═CH C≡C 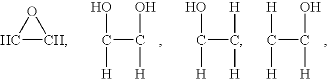
R⁵ means halogen or cyano,
R⁶, R7 together mean an additional bond or an oxygen atom,
G means a bicyclic or tricyclic aryl radical or the group 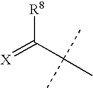
whereby R⁸ means hydrogen, fluorine, chlorine, bromine, cyano, C₁-C₂₀-alkyl, aryl, C₇-C₂₀-aralkyl, which can all be substituted,
X means an oxygen atom, two alkoxy groups OR¹⁹, a C₂-C₁₀-alkylene-α,ω-dioxy group, which can be straight-chain or branched, H/OR⁹
or a grouping CR¹⁰R¹¹,
whereby
R¹⁹ stands for a C₁-C₂₀-alkyl radical,
R⁹ stands for hydrogen or a protective group PGX,
R¹⁰, R¹¹ are the same or different and stand for hydrogen, a C₁-C₂₀ alkyl, aryl, C₇-C₂₀-aralkyl radical or R¹⁰ and R¹¹ together with the methylene carbon atom together stand for a 5- to 7-membered carbocyclic ring,
L means oxygen or NR²², whereby R²² is a hydrogen atom or a C₁-C₂₀-alkyl radical,
Y means an oxygen atom or two hydrogen atoms,
Z means an oxygen atom or H/OR¹²,
whereby
R¹² means hydrogen or a protective group PGz.

2. Epothilone derivatives according to claim 1, in which R^{1a} and R^{1b} each stand for a methyl group or together for an ethylene or trimethylene group.

3. Epothilone derivatives according to claim 1, in which Z stands for an oxygen atom.

4. Epothilone derivatives according to claim 1, in which the two substituents R^{2a} and R^{2b} are selected such that one stands for a hydrogen atom and the other stands for a methyl, ethyl, propyl, butyl, benzyl, allyl, homoallyl, propargyl or homopropargyl group.

5. Epothilone derivatives according to claim 1, in which R³ stands for a hydrogen atom.

6. Epothilone derivatives according to claim 1, in which R⁴ stands for a methyl, ethyl, propyl, butyl or benzyl group.

7. Epothilone derivatives according to claim 1, in which D stands for an oxygen atom and E stands for a methylene group, or D and E together stand for an ethylene group.

8. Epothilone derivatives according to claim 1, in which R⁵ stands for a fluorine, chlorine or bromine atom.

9. Epothilone derivatives according to claim 1, in which G stands for a bicyclic heteroaryl radical with at least one nitrogen atom.

10. Epothilone derivatives according to claim 9, in which the bicyclic heteroaryl radical is a 2-methyl-5-benzothiazolyl radical or 2-methyl-5-benzoxazolyl radical.

11. Epothilone derivatives according to claim 1, in which G stands for the grouping X═C(R⁸)— and R⁸ means a hydrogen, fluorine, chlorine or bromine atom or a methyl group and X means a group ═CR¹⁰R¹¹, in which R¹⁰ stands for a hydrogen atom, and R¹¹ stands for a heteroaryl-radical or vice versa.

12. Epothilone derivatives according to claim 11, in which the heteroaryl radical is a 2-methyl-4-thiazolyl, 2-pyridyl or 2-methyl-4-oxazolyl radical.

13. Epothilone derivatives according to claim 1, in which L is an oxygen atom or a nitrogen group —NR²²— with R²² in the meaning of a hydrogen atom or a methyl or ethyl group.

14. Epothilone derivatives according to claim 13, in which Y stands for an oxygen atom.

15. Epothilone derivatives according to claim 1, namely
(4S,7R,8S,9S,13E/Z,16S(E))-4,8-Dihydroxy-13-fluoro-16-(1-methyl-2-(2-methyl-4-thiazolyl)ethenyl)-1,11-dioxa-5,5,7,9-tetramethyl-cyclohexadec-13-ene-2,6-dione
(1S/R,3S(E),7S,10R,11S,12S,16S/R)-16-Fluoro-7,11-dihydroxy-3-(1-methyl-2-(2-methyl-4-thiazolyl)ethenyl)-8,8,10,12-tetramethyl-4,14,17-trioxabicyclo[14.1.0]heptadecane-5,9-dione
(4S,7R,8S,9S,13E/Z,16S(E))-4,8-Dihydroxy-13-chloro-16-(1-methyl-2-(2-methyl-4-thiazolyl)ethenyl)-1,11-dioxa-5,5,7,9-tetraamethyl-cyclohexadec-13-ene-2,6-dione
(1S/R,3S(E),7S,10R,11S,12S,16S/R)-16-Chloro-7,11-dihydroxy-3-(1-methyl-2-(2-methyl-4-thiazolyl)ethenyl)-8,8,10,12-tetramethyl-4,14,17-trioxabicyclo[14.1.0]heptadecane-5,9-dione
(4S,7R,8S,9S,13E/Z,16S(E))-4,8-Dihydroxy-13-cyano-16-(1-methyl-2-(2-methyl-4-thiazolyl)ethenyl)-1,11-dioxa-5,5,7,9-tetraamethyl-cyclohexadec-13-ene-2,6-dione
(1S/R,3 S(E),7S,10R,11S,12S,16S/R)-16-Cyano-7,11-dihydroxy-3-(1-methyl-2-(2-methyl-4-thiazolyl)ethenyl)-8,8,10,12-tetramethyl-4,14,17-trioxabicyclo[14.1.0]heptadecane-5,9-dione
(4S,7R,8S,9S,13E/Z,16S(E))-4,8-Dihydroxy-7-ethyl-13-fluoro-16-(1-methyl-2-(2-methyl-4-thiazolyl)ethenyl)-1,11-dioxa-5,5,9-trimethyl-cyclohexadec-13-ene-2,6-dione
(1S/R,3S(E),7S,10R,11 S,12S,16S/R)-16-Fluoro-10-ethyl-7,11-dihydroxy-3-(1-methyl-2-(2-methyl-4-thiazolyl)ethenyl)-8,8,12-trimethyl-4,14,17-trioxabicyclo[14.1.0]heptadecane-5,9-dione
(4S,7R,8S,9S,13E/Z,16S(E))-4,8-Dihydroxy-7-allyl-13-chloro-16-(1-methyl-2-(2-methyl-4-thiazolyl)ethenyl)-1,11-dioxa-5,5,9-trimethyl-cyclohexadec-13-ene-2,6-dione
(1S/R,3 S(E),7S,10R,11S,12S,16S/R)-16-Chloro-10-allyl-7,11-dihydroxy-3-(1-methyl-2-(2-methyl-4-thiazolyl)ethenyl)-8,8,12-trimethyl-4,14,17-trioxabicyclo [14.1.0]heptadecane-5,9-dione
(4S,7R,8S,9S,13E/Z,16S(E))-4,8-Dihydroxy-13-chloro-16-(1-methyl-2-(2-pyridyl)ethenyl)-1,11-dioxa-5,5,7,9-tetraamethyl-cyclohexadec-13-ene-2,6-dione
(1S/R,3 S(E),7S,10R,11S,12S,16S/R)-16-Chloro-7,11-dihydroxy-3-(1-methyl-2-(2-pyridyl)ethenyl)-8,8,10,12-tetramethyl-4,14,17-trioxabicyclo[14.1.0]heptadecane-5,9-dione
(4S,7R,8S,9S,13E/Z,16S(Z))-4,8-Dihydroxy-13-chloro-16-(1-fluoro-2-(2-methyl-4-thiazolyl)ethenyl)-1,11-dioxa-5,5,7,9-tetraamethyl-cyclohexadec-13-ene-2,6-dione
(1S/R,3S(Z),7S,10R,11S,12S,16S/R)-16-Chloro-7,11-dihydroxy-3-(1-fluoro-2-(2-methyl-4-thiazolyl)ethenyl)-8,8,10,12-tetramethyl-4,14,17-trioxabicyclo[14.1.0]heptadecane-5,9-dione
(4S,7R,8S,9S,13E/Z,16S(Z))-4,8-Dihydroxy-13-chloro-16-(1-chloro-2-(2-methyl-4-thiazolyl)ethenyl)-1,11-dioxa-5,5,7,9-tetraamethyl-cyclohexadec-13-ene-2,6-dione
(1S/R,3 S(Z),7S,10R,11S,12S,16S/R)-16-Chloro-7,11-dihydroxy-3-(1-chloro-2-(2-methyl-4-thiazolyl)ethenyl)-8,8,10,12-tetramethyl-4,14,17-trioxabicyclo[14.1.0]heptadecane-5,9-dione
(4S,7R,8S,9S,13E/Z,16S(Z))-4,8-Dihydroxy-13-chloro-16-(1-fluoro-2-(2-methyl-4-thiazolyl)ethenyl)-5,5-trimethylene-1,11-dioxa-7,9-dimethyl-cyclohexadec-13-ene-2,6-dione
(1S/R,3S(Z),7S,10R,11S,12S,16S/R)-16-Chloro-7,11-dihydroxy-3-(1-fluoro-2-(2-methyl-4-thiazolyl)ethenyl)-8,8-trimethylene-10,12-dimethyl-4,14,17-trioxabicyclo[14.1.0]heptadecane-5,9-dione
(4S,7R,8S,9S,13E/Z,16S(Z))-4,8-Dihydroxy-13-chloro-16-(1-chloro-2-(2-methyl-4-thiazolyl)ethenyl)-5,5-trimethylene-1,11-dioxa-7,9-dimethyl-cyclohexadec-13-ene-2,6-dione
(1S/R,3S(Z),7S,10R,11S,12S,16S/R)-16-Chloro-7,11-dihydroxy-3-(1-chloro-2-(2-methyl-4-thiazolyl)ethenyl)-8,8-trimethylene-10,12-dimethyl-4,14,17-trioxabicyclo [14.1.0]heptadecane-5,9-dione
(4S,7R,8S,9S,13E/Z,16S(Z))-4,8-Dihydroxy-13-chloro-16-(1-fluoro-2-(2-methyl-4-oxazolyl)ethenyl)-1,11-dioxa-5,5,7,9-tetraamethyl-cyclohexadec-13-ene-2,6-dione
(1S/R,3S(Z),7S,10R,11S,12S,16S/R)-16-Chloro-7,11-dihydroxy-3-(1-fluoro-2-(2-methyl-4-oxazolyl)ethenyl)-8,8,10,12-tetramethyl-4,14,17-trioxabicyclo [14.1.0]heptadecane-5,9-dione
(4S,7R,8S,9S,13E/Z,16S(Z))-4,8-Dihydroxy-13-chloro-16-(1-fluoro-2-(2-pyridyl)ethenyl)-1,11-dioxa-5,5,7,9-tetraamethyl-cyclohexadec-13-ene-2,6-dione
(1S/R,3S(Z),7S,10R,11S,12S,16S/R)-16-Chloro-7,11-dihydroxy-3-(1-fluoro-2-(2-pyridyl)ethenyl)-8,8,10,12-tetramethyl-4,14,17-trioxabicyclo[14.1.0]heptadecane-5,9-dione
(4S,7R,8S,9S,13E/Z,16S(Z))-4,8-Dihydroxy-7-ethyl-13-chloro-16-(1-chloro-2-(2-pyridyl)ethenyl)-1,11-dioxa-5,5,9-trimethyl-cyclohexadec-13-ene-2,6-dione
(1S/R,3S(Z),7S,10R,11S,12S,16S/R)-16-Chloro-7,11-dihydroxy-7-ethyl-3-(1-chloro-2-(2-pyridyl)ethenyl)-8,8,12-trimethyl-4,14,17-trioxabicyclo[14.1.0]heptadecane-5,9-dione
(4S,7R,8S,9S,13E/Z,16S)-4,8-Dihydroxy-13-fluoro-16-(2-methyl-5-benzothiazolyl)-1,11-dioxa-5,5,7,9-tetramethyl-cyclohexadec-13-ene-2,6-dione
(1S/R,3S,7S,10R,11S,12S,16S/R)-16-Fluoro-7,11-dihydroxy-3-(2-methyl-5-benzothiazolyl)-8,8,10,12-tetramethyl-4,14,17-trioxabicyclo[14.1.0]heptadecane-5,9-dione
(4S,7R,8S,9S,13E/Z,16S)-4,8-Dihydroxy-13-chloro-16-(2-methyl-5-benzothiazolyl)-1,11-dioxa-5,5,7,9-tetraamethyl-cyclohexadec-13-ene-2,6-dione
(1S/R,3S,7S,10R,11S,12S,16S/R)-16-Chloro-7,11-dihydroxy-3-(2-methyl-5-benzothiazolyl)-8,8,10,12-tetramethyl-4,14,17-trioxabicyclo[14.1.0]heptadecane-5,9-dione
(4S,7R,8S,9S,13E/Z,16S)-4,8-Dihydroxy-13-cyano-16-(2-methyl-5-benzothiazolyl)-1,11-dioxa-5,5,7,9-tetraamethyl-cyclohexadec-13-ene-2,6-dione
(1S/R,3S,7S,10R,11S,12S,16S/R)-16-Cyano-7,11-dihydroxy-3-(2-methyl-5-benzothiazolyl)-8,8,10,12-tetramethyl-4,14,17-trioxabicyclo [14.1.0]heptadecane-5,9-dione
(4S,7R,8S,9S,13E/Z,16S)-4,8-Dihydroxy-7-ethyl-13-chloro-16-(2-methyl-5-benzothiazolyl)-1,11-dioxa-5,5,9-trimethyl-cyclohexadec-13-ene-2,6-dione
(1S/R,3S,7S,10R,11S,12S,16S/R)-16-Chloro-7,11-dihydroxy-10-ethyl-3-(2-methyl-5-benzothiazolyl)-8,8,12-trimethyl-4,14,17-trioxabicyclo[14.1.0]heptadecane-5,9-dione
(4S,7R,8S,9S,13E/Z,16S)-4,8-Dihydroxy-7-allyl-13-chloro-16-(2-methyl-5-benzothiazolyl)-1,11-dioxa-5,5,9-trimethyl-cyclohexadec-13-ene-2,6-dione
(1S/R,3S,7S,10R,11S,12S,16S/R)-16-Chloro-7,11-dihydroxy-10-allyl-3-(2-methyl-5-benzothiazolyl)-8,8,12-trimethyl-4,14,17-trioxabicyclo [14.1.0]heptadecane-5,9-dione
(4S,7R,8S,9S,13 E/Z,16S)-4,8-Dihydroxy-13-fluoro-16-(2-methyl-5-benzoxazolyl)-1,11-dioxa-5,5,7,9-tetramethyl-cyclohexadec-13-ene-2,6-dione
(1S/R,3S,7S,10R,11S,12S,16S/R)-16-Fluoro-7,11-dihydroxy-3-(2-methyl-5-benzoxazolyl)-8,8,10,12-tetramethyl-4,14,17-trioxabicyclo[14.1.0]heptadecane-5,9-dione
(4S,7R,8S,9S,13E/Z,16S)-4,8-Dihydroxy-13-chloro-16-(2-methyl-5-benzoxazolyl)-1,11-dioxa-5,5,7,9-tetraamethyl-cyclohexadec-13-ene-2,6-dione
(1S/R,3S,7S,10R,11S,12S,16S/R)-16-Chloro-7,11-dihydroxy-3-(2-methyl-5-benzoxazolyl)-8,8,10,12-tetramethyl-4,14,17-trioxabicyclo[14.1.0]heptadecane-5,9-dione
(4S,7R,8S,9S,13E/Z,16S(E))-4,8-Dihydroxy-13-chloro-16-(1-methyl-2-(2-methyl-4-thiazolyl)ethenyl)-1-oxa-5,5,7,9-tetraamethyl-cyclohexadec-13-ene-2,6-dione
(1S/R,3S(E),7S,10R,11S,12S,16S/R)-16-Chloro-7,11-dihydroxy-3-(1-methyl-2-(2-methyl-4-thiazolyl)ethenyl)-8,8,10,12-tetramethyl-4,17-dioxabicyclo[14.1.0]heptadecane-5,9-dione
(4S,7R,8S,9S,13E/Z,16S(E))-4,8-Dihydroxy-13-cyano-16-(1-methyl-2-(2-methyl-4-thiazolyl)ethenyl)-1-oxa-5,5,7,9-tetraamethyl-cyclohexadec-13-ene-2,6-dione
(1S/R,3S(E),7S,10R,11S,12S,16S/R)-16-Cyano-7,11-dihydroxy-3-(1-methyl-2-(2-methyl-4-thiazolyl)ethenyl)-8,8,10,12-tetramethyl-4,17-dioxabicyclo[14.1.0]heptadecane-5,9-dione
(4S,7R,8S,9S,13E/Z,16S(E))-4,8-Dihydroxy-7-ethyl-13-fluoro-16-(1-methyl-2-(2-methyl-4-thiazolyl)ethenyl)-1-oxa-5,5,9-trimethyl-cyclohexadec-13-ene-2,6-dione
(1S/R,3S(E),7S,10R,1 S,12S,16S/R)-16-Fluoro-1 O-ethyl-7,11-dihydroxy-3-(1-methyl-2-(2-methyl-4-thiazolyl)ethenyl)-8,8,12-trimethyl-4,17-dioxabicyclo[14.1.0]heptadecane-5,9-dione
(4S,7R,8S,9S,13E/Z,16S(E))-4,8-Dihydroxy-7-allyl-13-chloro-16-(1-methyl-2-(2-methyl-4-thiazolyl)ethenyl)-1-oxa-5,5,9-trimethyl-cyclohexadec-13-ene-2,6-dione
(1S/R,3S(E),7S,10R,11S,12S,16S/R)-16-Chloro-10-allyl-7,11-dihydroxy-3-(1-methyl-2-(2-methyl-4-thiazolyl)ethenyl)-8,8,12-trimethyl-4,17-dioxabicyclo[14.1.0]heptadecane-5,9-dione
(4S,7R,8S,9S,13E/Z,16S(E))-4,8-Dihydroxy-13-chloro-16-(1-methyl-2-(2-pyridyl)ethenyl)-1-oxa-5,5,7,9-tetraamethyl-cyclohexadec-13-ene-2,6-dione
(1S/R,3S(E),7S,10R,11S,12S,16S/R)-16-Chloro-7,11-dihydroxy-3-(1-methyl-2-(2-pyridyl)ethenyl)-8,8,10,12-tetramethyl-4,17-dioxabicyclo[14.1.0]heptadecane-5,9-dione
(4S,7R,8S,9S,13E/Z,16S(Z))-4,8-Dihydroxy-13-chloro-16-(1-fluoro-2-(2-pyridyl)ethenyl)-1-oxa-5,5,7,9-tetraamethyl-cyclohexadec-13-ene-2,6-dione
(1S/R,3S(Z),7S,10R,11S,12S,16S/R)-16-Chloro-7,11-dihydroxy-3-(1-fluoro-2-(2-pyridyl)ethenyl)-8,8,10,12-tetramethyl-4,17-dioxabicyclo[14.1.0]heptadecane-5,9-dione
(4S,7R,8S,9S,13E/Z,16S(Z))-4,8-Dihydroxy-7-ethyl-13-chloro-16-(1-chloro-2-(2-pyridyl)ethenyl)-1-oxa-5,5,9-trimethyl-cyclohexadec-13-ene-2,6-dione
(1S/R,3S(Z),7S,10R,11S,12S,16S/R)-16-Chloro-7,11-dihydroxy-7-ethyl-3-(1-chloro-2-(2-pyridyl)ethenyl)-8,8,12-trimethyl-4,17-dioxabicyclo[14.1.0]heptadecane-5,9-dione
(4S,7R,8S,9S,13E/Z,16S(Z))-4,8-Dihydroxy-13-chloro-16-(1-chloro-2-(2-methyl-4-thiazolyl)ethenyl)-1-oxa-5,5,7,9-tetraamethyl-cyclohexadec-13-ene-2,6-dione
(1S/R,3S(Z),7S,10R,11S,12S,16S/R)-16-Chloro-7,11-dihydroxy-3-(1-chloro-2-(2-methyl-4-thiazolyl)ethenyl)-8,8,10,12-tetramethyl-4,17-dioxabicyclo [14.1.0]heptadecane-5,9-dione
(4S,7R,8S,9S,13E/Z,16S(Z))-4,8-Dihydroxy-13-chloro-16-(1-fluoro-2-(2-methyl-4-thiazolyl)ethenyl)-5,5-trimethylen-1-oxa-7,9-dimethyl-cyclohexadec-13-ene-2,6-dione
(1S/R,3S(Z),7S,10R,11S,12S,16S/R)-16-Chloro-7,11-dihydroxy-3-(1-fluoro-2-(2-methyl-4-thiazolyl)ethenyl)-8,8-trimethylene-10,12-dimethyl-4,17-dioxabicyclo[14.1.0]heptadecane-5,9-dione
(4S,7R,8S,9S,13E/Z,16S(Z))-4,8-Dihydroxy-13-chloro-16-(1-chloro-2-(2-methyl-4-thiazolyl)ethenyl)-5,5-trimethylen-1-oxa-7,9-dimethyl-cyclohexadec-13-ene-2,6-dione
(1S/R,3S(Z),7S,10R,11 S,12S,16S/R)-16-Chloro-7,11-dihydroxy-3-(1-chloro-2-(2-methyl-4-thiazolyl)ethenyl)-8,8-trimethylene-10,12-dimethyl-4,17-dioxabicyclo[14.1.0]heptadecane-5,9-dione
(4S,7R,8S,9S,13E/Z,16S)-4,8-Dihydroxy-13-fluoro-16-(2-methyl-5-benzothiazolyl)-1-oxa-5,5,7,9-tetramethyl-cyclohexadec-13-ene-2,6-dione
(1S/R,3S,7S,10R,11S,12S,16S/R)-16-Fluoro-7,11-dihydroxy-3-(2-methyl-5-benzothiazolyl)-8,8,10,12-tetramethyl-4,17-dioxabicyclo[14.1.0]heptadecane-5,9-dione
(4S,7R,8S,9S,13E/Z,16S)-4,8-Dihydroxy-13-chloro-16-(2-methyl-5-benzothiazolyl)-1-oxa-5,5,7,9-tetraamethyl-cyclohexadec-13-ene-2,6-dione
(1S/R,3S,7S,10R,11S,12S,16S/R)-16-Chloro-7,11-dihydroxy-3-(2-methyl-5-benzothiazolyl)-8,8,10,12-tetramethyl-4,17-dioxabicyclo[14.1.0]heptadecane-5,9-dione
(4S,7R,8S,9S,13E/Z,16S)-4,8-Dihydroxy-7-ethyl-13-chloro-16-(2-methyl-5-benzothiazolyl)-1-oxa-5,5,9-trimethyl-cyclohexadec-13-ene-2,6-dione
(1S/R,3S,7S,10R,11S,12S,16S/R)-16-Chloro-7,11-dihydroxy-10-ethyl-3-(2-methyl-5-benzothiazolyl)-8,8,12-trimethyl-4,17-dioxabicyclo[14.1.0]heptadecane-5,9-dione
(4S,7R,8S,9S,13E/Z,16S)-4,8-Dihydroxy-7-allyl-13-chloro-16-(2-methyl-5-benzothiazolyl)-1-oxa-5,5,9-trimethyl-cyclohexadec-13-ene-2,6-dione
(1S/R,3S,7S,10R,11S,12S,16S/R)-16-Chloro-7,11-dihydroxy-10-allyl-3-(2-methyl-5-benzothiazolyl)-8,8,12-trimethyl-4,17-dioxabicyclo[14.1.0]heptadecane-5,9-dione
(4S,7R,8S,9S,13E/Z,16S(E))-4,8-Dihydroxy-13-fluoro-16-(1-methyl-2-(2-methyl-4-thiazolyl)ethenyl)-1-aza-11-oxa-5,5,7,9-tetramethyl-cyclohexadec-13-ene-2,6-dione
(1S/R,3S(E),7S,10R,11S,12S,16S/R)-16-Fluoro-7,11-dihydroxy-3-(1-methyl-2-(2-methyl-4-thiazolyl)ethenyl)-8,8,10,12-tetramethyl-4-aza-14,17-dioxabicyclo[14.1.0]heptadecane-5,9-dione
(4S,7R,8S,9S,13E/Z,16S(E))-4,8-Dihydroxy-13-chloro-16-(1-methyl-2-(2-methyl-4-thiazolyl)ethenyl)-1-aza-11-oxa-5,5,7,9-tetraamethyl-cyclohexadec-13-ene-2,6-dione
(1S/R,3S(E),7S,10R,11S,12S,16S/R)-16-Chloro-7,11-dihydroxy-3-(1-methyl-2-(2-methyl-4-thiazolyl)ethenyl)-8,8,10,12-tetramethyl-4-aza-14,17-dioxabicyclo [14.1.0]heptadecane-5,9-dione
(4S,7R,8S,9S,13E/Z,16S(E))-4,8-Dihydroxy-13-cyano-16-(1-methyl-2-(2-methyl-4-thiazolyl)ethenyl)-1-aza-1′-oxa-5,5,7,9-tetraamethyl-cyclohexadec-13-ene-2,6-dione
(1S/R,3S(E),7S,10R,11S,12S,16S/R)-16-Cyano-7,11-dihydroxy-3-(1-methyl-2-(2-methyl-4-thiazolyl)ethenyl)-8,8,10,12-tetramethyl-4-aza-14,17-dioxabicyclo[14.1.0]heptadecane-5,9-dione
(4S,7R,8S,9S,13E/Z,16S(E))-4,8-Dihydroxy-13-cyano-16-(1-methyl-2-(2-methyl-4-thiazolyl)ethenyl)-1-aza-11-oxa-5,5,7,9-tetraamethyl-cyclohexadec-13-ene-2,6-dione
(1S/R,3 S(E),7S,10R,11 S,12S,16S/R)-16-Cyano-7,11-dihydroxy-3-(1-methyl-2-(2-methyl-4-thiazolyl)ethenyl)-8,8,10,12-tetramethyl-4-aza-14,17-dioxabicyclo[14.1.0]heptadecane-5,9-dione
(4S,7R,8S,9S,13E/Z,16S(E))-4,8-Dihydroxy-7-ethyl-13-fluoro-16-(1-methyl-2-(2-methyl-4-thiazolyl)ethenyl)-1-aza-11-oxa-5,5,9-trimethyl-cyclohexadec-13-ene-2,6-dione
(1S/R,3S(E),7S,10R,11S,12S,16S/R)-16-Fluoro-10-ethyl-7,11-dihydroxy-3-(1-methyl-2-(2-methyl-4-thiazolyl)ethenyl)-8,8,12-trimethyl-4-aza-14,17-dioxabicyclo[14.1.0]heptadecane-5,9-dione
(4S,7R,8S,9S,13E/Z,16S(E))-4,8-Dihydroxy-7-allyl-13-chloro-16-(1-methyl-2-(2-methyl-4-thiazolyl)ethenyl)-1-aza-11-oxa-5,5,9-trimethyl-cyclohexadec-13-ene-2,6-dione
(1S/R,3S(E),7S,10R,11S,12S,16S/R)-16-Chloro-10-allyl-7,11-dihydroxy-3-(1-methyl-2-(2-methyl-4-thiazolyl)ethenyl)-8,8,12-trimethyl-4-aza-14,17-dioxabicyclo[14.1.0]heptadecane-5,9-dione
(4S,7R,8S,9S,13E/Z,16S(E))-4,8-Dihydroxy-13-chloro-16-(1-methyl-2-(2-pyridyl)ethenyl)-1-aza-11-oxa-5,5,7,9-tetraamethyl-cyclohexadec-13-ene-2,6-dione
(1S/R,3S(E),7S,10R,11S,12S,16S/R)-16-Chloro-7,11-dihydroxy-3-(1-methyl-2-(2-pyridyl)ethenyl)-8,8,10,12-tetramethyl-4-aza-14,17-dioxabicyclo[14.1.0]heptadecane-5,9-dione
(4S,7R,8S,9S,13E/Z,16S(Z))-4,8-Dihydroxy-13-chloro-16-(1-fluoro-2-(2-methyl-4-thiazolyl)ethenyl)-1-aza-11-oxa-5,5,7,9-tetraamethyl-cyclohexadec-13-ene-2,6-dione
(1S/R,3S(Z),7S,10R,11S,12S,16S/R)-16-Chloro-7,11-dihydroxy-3-(1-fluoro-2-(2-methyl-4-thiazolyl)ethenyl)-8,8,10,12-tetramethyl-4-aza-14,17-dioxabicyclo[14.1.0]heptadecane-5,9-dione
(4S,7R,8S,9S,13E/Z,16S(Z))-4,8-Dihydroxy-13-chloro-16-(1-chloro-2-(2-methyl-4-thiazolyl)ethenyl)-1-aza-11-oxa-5,5,7,9-tetraamethyl-cyclohexadec-13-ene-2,6-dione
(1 S/R,3S(Z),7S,10R,11S,12S,16S/R)-16-Chloro-7,11-dihydroxy-3-(1-chloro-2-(2-methyl-4-thiazolyl)ethenyl)-8,8,10,12-tetramethyl-4-aza-14,17-dioxabicyclo[14.1.0]heptadecane-5,9-dione
(4S,7R,8S,9S,13E/Z,16S(Z))-4,8-Dihydroxy-13-chloro-16-(1-fluoro-2-(2-methyl-4-thiazolyl)ethenyl)-5,5-trimethylen-1-aza-11-oxa-7,9-dimethyl-cyclohexadec-13-ene-2,6-dione
(1S/R,3S(Z),7S,10R,11S,12S,16S/R)-16-Chloro-7,11-dihydroxy-3-(1-fluoro-2-(2-methyl-4-thiazolyl)ethenyl)-8,8-trimethylene-10,12-dimethyl-4-aza-14,17-dioxabicyclo[14.1.0]-heptadecane-5,9-dione
(4S,7R,8S,9S,13E/Z,16S(Z))-4,8-Dihydroxy-13-chloro-16-(1-chloro-2-(2-methyl-4-thiazolyl)ethenyl)-5,5-trimethylen-1-aza-11-oxa-7,9-dimethyl-cyclohexadec-13-ene-2,6-dione
(1S/R,3S(Z),7S,10R,11S,12S,16S/R)-16-Chloro-7,11-dihydroxy-3-(1-chloro-2-(2-methyl-4-thiazolyl)ethenyl)-8,8-trimethylene-10,12-dimethyl-4-aza-14,17-dioxabicyclo[14.1.0]-heptadecane-5,9-dione
(4S,7R,8S,9S,13E/Z,16S(Z))-4,8-Dihydroxy-13-chloro-16-(1-fluoro-2-(2-methyl-4-oxazolyl)ethenyl)-1-aza-11-oxa-5,5,7,9-tetraamethyl-cyclohexadec-13-ene-2,6-dione
(1S/R,3 S(Z),7S,10R,11S,12S,16S/R)-16-Chloro-7,11-dihydroxy-3-(1-fluoro-2-(2-methyl-4-oxazolyl)ethenyl)-8,8,10,12-tetramethyl-4-aza-14,17-dioxabicyclo[14.1.0]heptadecane-5,9-dione
(4S,7R,8S,9S,13E/Z,16S(Z))-4,8-Dihydroxy-13-chloro-16-(1-fluoro-2-(2-pyridyl)ethenyl)-1-aza-11-oxa-5,5,7,9-tetraamethyl-cyclohexadec-13-ene-2,6-dione
(1S/R,3 S(Z),7S,10R,11S,12S,16S/R)-16-Chloro-7,11-dihydroxy-3-(1-fluoro-2-(2-pyridyl)ethenyl)-8,8,10,12-tetramethyl-4-aza-14,17-dioxabicyclo [14.1.0]heptadecane-5,9-dione
(4S,7R,8S,9S,13E/Z,16S(Z))-4,8-Dihydroxy-7-ethyl-13-chloro-16-(1-chloro-2-(2-pyridyl)ethenyl)-1-aza-11-oxa-5,5,9-trimethyl-cyclohexadec-13-ene-2,6-dione
(1S/R,3S(Z),7S,10R,11S,12S,16S/R)-16-Chloro-7,11-dihydroxy-7-ethyl-3-(1-chloro-2-(2-pyridyl)ethenyl)-8,8,12-trimethyl-4-aza-14,17-dioxabicyclo[14.1.0]heptadecane-5,9-dione
(4S,7R,8S,9S,13E/Z,16S)-4,8-Dihydroxy-13-fluoro-16-(2-methyl-5-benzothiazolyl)-1-aza-11-oxa-5,5,7,9-tetramethyl-cyclohexadec-13-ene-2,6-dione
(1S/R,3S,7S,10R,11S,12S,16S/R)-16-Fluoro-7,11-dihydroxy-3-(2-methyl-5-benzothiazolyl)-8,8,10,12-tetramethyl-4-aza-14,17-dioxabicyclo[14.1.0]heptadecane-5,9-dione
(4S,7R,8S,9S,13E/Z,16S)-4,8-Dihydroxy-13-chloro-16-(2-methyl-5-benzothiazolyl)-1-aza-11-oxa-5,5,7,9-tetraamethyl-cyclohexadec-13-ene-2,6-dione
(1S/R,3S,7S,10R,11S,12S,16S/R)-16-Chloro-7,11-dihydroxy-3-(2-methyl-5-benzothiazolyl)-8,8,10,12-tetramethyl-4-aza-14,17-dioxabicyclo[14.1.0]heptadecane-5,9-dione
(4S,7R,8S,9S,13E/Z,16S)-4,8-Dihydroxy-13-cyano-16-(2-methyl-5-benzothiazolyl)-1-aza-11-oxa-5,5,7,9-tetraamethyl-cyclohexadec-13-ene-2,6-dione
(1S/R,3S,7S,10R,11S,12S,16S/R)-16-Cyano-7,11-dihydroxy-3-(2-methyl-5-benzothiazolyl)-8,8,10,12-tetramethyl-4-aza-14,17-dioxabicyclo[14.1.0]heptadecane-5,9-dione
(4S,7R,8S,9S,13E/Z,16S)-4,8-Dihydroxy-7-ethyl-13-chloro-16-(2-methyl-5-benzothiazolyl)-1-aza-11-oxa-5,5,9-trimethyl-cyclohexadec-13-ene-2,6-dione
(1S/R,3S,7S,10R,11S,12S,16S/R)-16-Chloro-7,11-dihydroxy-10-ethyl-3-(2-methyl-5-benzothiazolyl)-8,8,12-trimethyl-4-aza-14,17-dioxabicyclo[14.1.0]heptadecane-5,9-dione
(4S,7R,8S,9S,13E/Z,16S)-4,8-Dihydroxy-7-allyl-13-chloro-16-(2-methyl-5-benzothiazolyl)-1-aza-11-oxa-5,5,9-trimethyl-cyclohexadec-13-ene-2,6-dione
(1S/R,3S,7S,10R,11 S,12S,16S/R)-16-Chloro-7,11-dihydroxy-10-allyl-3-(2-methyl-5-benzothiazolyl)-8,8,12-trimethyl-4-aza-14,17-dioxabicyclo[14.1.0]heptadecane-5,9-dione
(4S,7R,8S,9S,13E/Z,16S)-4,8-Dihydroxy-13-fluoro-16-(2-methyl-5-benzoxazolyl)-1-aza-11-oxa-5,5,7,9-tetrarnethyl-cyclohexadec-13-ene-2,6-dione
(1S/R,3S,7S,10R,11S,12S,16S/R)-16-Fluoro-7,11-dihydroxy-3-(2-methyl-5-benzoxazolyl)-8,8,10,12-tetramethyl-4-aza-14,17-dioxabicyclo[14.1.0]heptadecane-5,9-dione
(4S,7R,8S,9S,13E/Z,16S)-4,8-Dihydroxy-13-chloro-16-(2-methyl-5-benzoxazolyl)-1-aza-11-oxa-5,5,7,9-tetraamethyl-cyclohexadec-13-ene-2,6-dione
(1S/R,3S,7S,10R,11S,12S,16S/R)-16-Chloro-7,11-dihydroxy-3-(2-methyl-5-benzoxazolyl)-8,8,10,12-tetramethyl-4-aza-14,17-dioxabicyclo [14.1.0]heptadecane-5,9-dione
(4S,7R,8S,9S,13E/Z,16S(E))-4,8-Dihydroxy-13-chloro-16-(1-methyl-2-(2-methyl-4-thiazolyl)ethenyl)-1-aza-5,5,7,9-tetraamethyl-cyclohexadec-13-ene-2,6-dione
(1S/R,3 S(E),7S,10R,11S,12S,16S/R)-16-Chloro-7,11-dihydroxy-3-(1-methyl-2-(2-methyl-4-thiazolyl)ethenyl)-8,8,10,12-tetramethyl-4-aza-17-oxabicyclo[14.1.0]heptadecane-5,9-dione
(4S,7R,8S,9S,13E/Z,16S(E))-4,8-Dihydroxy-7-ethyl-13-fluoro-16-(1-methyl-2-(2-methyl-4-thiazolyl)ethenyl)-1-aza-5,5,9-trimethyl-cyclohexadec-13-ene-2,6-dione
(1S/R,3 S(E),7S,10R,11S,12S,16S/R)-16-Fluoro-1 O-ethyl-7,11-dihydroxy-3-(1-methyl-2-(2-methyl-4-thiazolyl)ethenyl)-8,8,12-trimethyl-4-aza-17-oxabicyclo[14.1.0]heptadecane-5,9-dione
(4S,7R,8S,9S,13E/Z,16S(E))-4,8-Dihydroxy-7-allyl-13-chloro-16-(1-methyl-2-(2-methyl-4-thiazolyl)ethenyl)-1-aza-5,5,9-trimethyl-cyclohexadec-13-ene-2,6-dione
(1S/R,3S(E),7S,10R,11S,12S,16S/R)-16-Chloro-10-allyl-7,11-dihydroxy-3-(1-methyl-2-(2-methyl-4-thiazolyl)ethenyl)-8,8,12-trimethyl-4-aza-17-oxabicyclo[14.1.0]heptadecane-5,9-dione
(4S,7R,8S,9S,13E/Z,16S(E))-4,8-Dihydroxy-13-chloro-16-(1-methyl-2-(2-pyridyl)ethenyl)-1-aza-5,5,7,9-tetraamethyl-cyclohexadec-13-ene-2,6-dione
(1S/R,3S(E),7S,10R,11S,12S,16S/R)-16-Chloro-7,11-dihydroxy-3-(1-methyl-2-(2-pyridyl)ethenyl)-8,8,10,12-tetramethyl-4-aza-17-oxabicyclo [14.1.0]heptadecane-5,9-dione
(4S,7R,8S,9S,13E/Z,16S(Z))-4,8-Dihydrbxy-13-chloro-16-(1-fluoro-2-(2-pyridyl)ethenyl)-1-aza-5,5,7,9-tetraamethyl-cyclohexadec-13-ene-2,6-dione
(1S/R,3 S(Z),7S,10R,11S,12S,16S/R)-16-Chloro-7,11-dihydroxy-3-(1-fluoro-2-(2-pyridyl)ethenyl)-8,8,10,12-tetramethyl-4-aza-17-oxabicyclo[14.1.0]heptadecane-5,9-dione
(4S,7R,8S,9S,13E/Z,16S(Z))-4,8-Dihydroxy-7-ethyl-13-chloro-16-(1-chloro-2-(2-pyridyl)ethenyl)-1-aza-5,5,9-trimethyl-cyclohexadec-13-ene-2,6-dione
(1S/R,3 S(Z),7S,10R,11S,12S,16S/R)-16-Chloro-7,11-dihydroxy-7-ethyl-3-(1-chloro-2-(2-pyridyl)ethenyl)-8,8,12-trimethyl-4-aza-17-oxabicyclo[14.1.0]heptadecane-5,9-dione
(4S,7R,8S,9S,13 E/Z,16S(Z))-4,8-Dihydroxy-13-chloro-16-(1-chloro-2-(2-methyl-4-thiazolyl)ethenyl)-1-aza-5,5,7,9-tetraamethyl-cyclohexadec-13-ene-2,6-dione
(1S/R,3S(Z),7S,10R,11S,12S,16S/R)-16-Chloro-7,11-dihydroxy-3-(1-chloro-2-(2-methyl-4-thiazolyl)ethenyl)-8,8,10,12-tetramethyl-4-aza-17-oxabicyclo[14.1.0]heptadecane-5,9-dione
(4S,7R,8S,9S,13E/Z,16S(Z))-4,8-Dihydroxy-13-chloro-16-(1-fluoro-2-(2-methyl-4-thiazolyl)ethenyl)-5,5-trimethylen-1-aza-7,9-dimethyl-cyclohexadec-13-ene-2,6-dione
(1S/R,3 S(Z),7S,10R,11S,12S,16S/R)-16-Chloro-7,11-dihydroxy-3-(1-fluoro-2-(2-methyl-4-thiazolyl)ethenyl)-8,8-trimethylene-10,12-dimethyl-4-aza-17-oxabicyclo[14.1.0]heptadecane-5,9-dione
(4S,7R,8S,9S,13E/Z,16S(Z))-4,8-Dihydroxy-13-chloro-16-(1-chloro-2-(2-methyl-4-thiazolyl)ethenyl)-5,5-trimethylen-1-aza-7,9-dimethyl-cyclohexadec-13-ene-2,6-dione
(1S/R,3S(Z),7S,10R,11S,12S,16S/R)-16-Chloro-7,11-dihydroxy-3-(1-chloro-2-(2-methyl-4-thiazolyl)ethenyl)-8,8-trimethylene-10,12-dimethyl-4-aza-17-oxabicyclo[14.1.0]heptadecane-5,9-dione
(4S,7R,8S,9S,13E/Z,16S)-4,8-Dihydroxy-13-fluoro-16-(2-methyl-5-benzothiazolyl)-1-aza-5,5,7,9-tetramethyl-cyclohexadec-13-ene-2,6-dione
(1S/R,3 S,7S,10R,11S,12S,16S/R)-16-Fluoro-7,11-dihydroxy-3-(2-methyl-5-benzothiazolyl)-8,8,10,12-tetramethyl-4-aza-17-oxabicyclo[14.1.0]heptadecane-5,9-dione
(4S,7R,8S,9S,13E/Z,16S)-4,8-Dihydroxy-13-chloro-16-(2-methyl-5-benzothiazolyl)-1-aza-5,5,7,9-tetraamethyl-cyclohexadec-13-ene-2,6-dione
(1S/R,3S,7S,10R,11S,12S,16S/R)-16-Chloro-7,11-dihydroxy-3-(2-methyl-5-benzothiazolyl)-8,8,10,12-tetramethyl-4-aza-17-oxabicyclo[14.1.0]heptadecane-5,9-dione
(4S,7R,8S,9S,13E/Z,16S)-4,8-Dihydroxy-13-cyano-16-(2-methyl-5-benzothiazolyl)-1-aza-5,5,7,9-tetraamethyl-cyclohexadec-13-ene-2,6-dione
(1S/R,3S,7S,10R,11S,12S,16S/R)-16-Cyano-7,11-dihydroxy-3-(2-methyl-5-benzothiazolyl)-8,8,10,12-tetramethyl-4-aza-17-oxabicyclo[14.1.0]heptadecane-5,9-dione
(4S,7R,8S,9S,13E/Z,16S)-4,8-Dihydroxy-7-ethyl-13-chloro-16-(2-methyl-5-benzothiazolyl)-1-aza-5,5,9-trimethyl-cyclohexadec-13-ene-2,6-dione
(1S/R,3S,7S,10R,11S,12S,16S/R)-16-Chloro-7,11-dihydroxy-10-ethyl-3-(2-methyl-5-benzothiazolyl)-8,8,12-trimethyl-4-aza-17-oxabicyclo[14.1.0]heptadecane-5,9-dione
(4S,7R,8S,9S,13E/Z,16S)-4,8-Dihydroxy-7-allyl-13-chloro-16-(2-methyl-5-benzothiazolyl)-1-aza-5,5,9-trimethyl-cyclohexadec-13-ene-2,6-dione
(1S/R,3S,7S,10R,11S,12S,16S/R)-16-Chloro-7,11-dihydroxy-10-allyl-3-(2-methyl-5-benzothiazolyl)-8,8,12-trimethyl-4-aza-17-oxabicyclo[14.1.0]heptadecane-5,9-dione

16. Intermediate products of general formula B-VIII.

17. Intermediate products of general formula B-XV.

18. Intermediate products of general formula AB 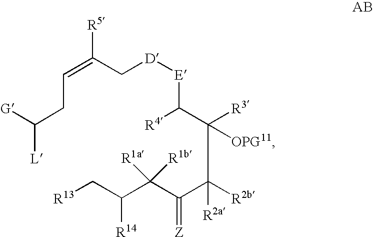
in which R^{1a′}, R^{1b′}, R^{2a′},R^{2b′}, R^{3′}, R^{4′}, R^{5′}, R¹³, R¹⁴, D′, E′, L′, G′ and Z have the meanings already mentioned.

19. Pharmaceutical preparations that contain at least one epothilone derivative of general formula I according to claim 1, as well as a pharmaceutically compatible vehicle.

20. Use of the epothilone derivatives of general formula I according to claim 1 for the production of pharmaceutical agents.
